(19) United States
(12) Patent Application Publication
(10) Pub. No.: US 2025/0084491 A1
(43) Pub. Date: Mar. 13, 2025

(54) COMPOSITIONS AND METHODS FOR CHARACTERIZING A COMPLEX BIOLOGICAL SAMPLE

(71) Applicant: The Broad Institute, Inc., Cambridge, MA (US)

(72) Inventors: Ashlee M. EARL, Cambridge, MA (US); Timothy J. STRAUB, Cambridge, MA (US); Hayden C. METSKY, Cambridge, MA (US)

(73) Assignee: The Broad Institute, Inc., Cambridge, MA (US)

(21) Appl. No.: 18/580,579

(22) PCT Filed: Jul. 15, 2022
(86) PCT No.: PCT/IB2022/056558

§ 371 Date: Jan. 18, 2024

Related U.S. Application Data
(60) Provisional application No. 63/223,185, filed on Jul. 19, 2021.

(51) Int. Cl. C12Q 1/689; C12Q 1/6806; C12Q 1/6837; G16B 25/20

(52) U.S. Cl. CPC C12Q 1/689; C12Q 1/6806; C12Q 1/6837; G16B 25/20

(57) ABSTRACT

The invention features compositions and methods that are useful for characterizing a complex biological sample.

### CROSS-REFERENCE TO RELATED APPLICATION

This application claims priority to and the benefit of U.S. Provisional Application No. 63/223, 185, filed Jul. 19, 2021, the entire contents of which are incorporated herein by reference.

### STATEMENT OF RIGHTS TO INVENTIONS MADE UNDER FEDERALLY SPONSORED RESEARCH

This invention was made with government support under Grant No. U19AI110818 awarded by the National Institutes of Health. The government has certain rights in the invention.

### SEQUENCE LISTING

This application contains a Sequence Listing which has been submitted electronically in XML format and is hereby incorporated by reference in its entirety. The Sequence Listing XML file, created on Jul. 6, 2022, is named 167741-029001PCT_SL.xml and is 840,431,311 bytes in size.

### BACKGROUND OF THE INVENTION

Clinically important microbes are often found at low abundance within complex communities, presenting a challenge for their investigation. For example, pathogenic Escherichia coli are commonly minor members of the human gut microbiome, found at an average relative abundance of less than 1%. These low-abundance organisms are difficult to characterize in their metagenomic context using sequencing approaches due to very low signal-to-noise, without massive costs for deep sequencing. Although some bacteria can be cultured, these approaches are slow and can lead to known biases. Furthermore, for many species, multiple conspecific strains are often present within the microbiome, including E. coli, further complicating isolation-based approaches, as well as their study within low-abundance sequence data. E. coli present in the gut constitutes a risk factor for urinary tract infection. Urinary tract infection (UTI) is one of the most common bacterial infections in the world, affecting over 150 million people annually. E. coli cause over 80% of all UTIs and can be remarkably diverse, with two strains sometimes sharing only approximately 50% of their genetic content. Many individuals suffering from UTI go on to develop recurrent UTIs, which are resistant to conventional therapies. Recurrent UTIs within the same individual are frequently associated with different E. coli strains. Current methods for analyzing complex biological samples from the gut and urinary tract microbiomes are inadequate. Thus, there is a need for improved methods to characterize low-abundance organisms in complex biological samples.

### SUMMARY OF THE INVENTION

As described below, the present invention features compositions and for characterizing complex biological samples (e.g., microbiomes).

In one aspect, the invention features a method of generating a set of probes. The method involves identifying a plurality of clusters of gene sequences derived from a set of genomes derived from organisms. The gene sequences within each cluster share at least about 25% nucleotide sequence identity. The method further involves generating a set of probes, where each probe within the set contains at least about 25 nucleotides and at least about 80% of said nucleotides are complementary to a target sequence present in the clusters of gene sequences. The probes collectively target at least about 10% of all gene sequences in a gene cluster.

A method for generating a set of probes for use in pan-genomic or pan-transcriptomic sequencing of polynucleotides derived from organisms present in a microbiome. The method involves identifying a plurality of orthogroup clusters of gene sequences within a set of genomes derived from the organisms, where the organism are present in a microbiome. The method further involves identifying a plurality of gene clusters within the orthogroup clusters, where the gene sequences within each of the gene clusters share at least about 80% nucleotide sequence identity. The method further involves generating a set of probes, where each probe within the set contains from about 50 to about 85 nucleotides, where each probe is complementary to at least about 25 base pairs of a gene sequence present in the gene clusters identified. The probes collectively cover at least about 50% of all gene sequences in the gene clusters, and set of probes excludes probes having 50 or more contiguous nucleotides that are identical to a set of reference sequences. Coverage of each probe is determined using a cover extension of about 20 bp.

In another aspect, the invention features a polynucleotide array containing the set of probes of any of the above aspects.

In another aspect, the invention features a set of probes suitable for use in the method of any of the above aspects, where the probes contain a set of sequences each sharing at least 80% nucleotide sequence identity along a span of at least about 65 nucleotides to a sequence selected from SEQ ID NOs: 1 to 892415.

In another aspect, the invention features a panel of probes containing probes generated according to the method of any one of the above aspects, or the set of probes of any one of the above aspects.

In another aspect, the invention features a method of characterizing a complex biological sample. The method involves contacting polynucleotides derived from the complex biological sample with the set of probes generated in any of the above aspects, or a subset of such probes, under conditions that permit hybridization of the probes to the polynucleotides, thereby forming polynucleotide/probe complexes, where each probe is coupled to a binding member. The method further involves contacting the polynucleotide/probe complexes with a capture molecule fixed to a solid support, where the capture molecule specifically binds the binding member of the probe, thereby enriching polynucleotides of the complex biological sample. The method further involves characterizing the enriched polynucleotides. In embodiments, the characterizing involves sequencing, qPCR, fluorescent imaging, fluorescence activated cell sorting (FACS), genotyping array, or a NanoString assay.

In another aspect, the invention features a method of characterizing a complex biological sample. The method involves contacting polynucleotides derived from a complex biological sample with the set of probes of any of the above aspects, or a subset of such probes, under conditions that permit hybridization of the probes to the polynucleotides, thereby forming polynucleotide/probe complexes, where each probe is coupled to a solid support, thereby enriching polynucleotides of the complex biological sample. The method further involves characterizing the enriched polynucleotides, where the characterization involves sequencing, qPCR, fluorescent imaging, FACS, genotyping array, or a NanoString assay.

In another aspect, the invention features a method for enrichment of polynucleotides derived from a complex biological sample. The method involves contacting polynucleotides derived from the complex biological sample with the set of probes of any of the above aspects, or a subset thereof, under conditions that permit hybridization of the set of probes to the polynucleotides, where each probe is coupled to a binding member and/or to a solid support, thereby forming polynucleotide/probe complexes. If the probe is not coupled to a solid support, the method further involves, contacting the polynucleotide/probe complex with a capture molecule fixed to a solid support, where the capture molecule specifically binds the binding member of the probe. The method provides for enriching the polynucleotides derived from the complex biological sample.

In another aspect, the invention features a set of enriched polynucleotide sequences obtained by the method of any of the above aspects.

In any of the above aspects, the complex biological sample contains polynucleotides derived from a host organism and an organism of interest, and the enriched polynucleotides are derived from the organism of interest.

In any of the above aspects, the enriched polynucleotides are derived from genes associated with a function of interest.

In any of the above aspects, the complex biological sample is an agricultural sample, biological sample, environmental sample, or food sample. In embodiments, the environmental sample is a drinking water sample, a river water sample, a wastewater treatment sample, a sample from a body of salt water, or a sample from a body of freshwater. In embodiments, the biological sample is a solid or liquid sample. In embodiments, the liquid sample is selected from one or more of a blood, breast milk, bone marrow, cerebrospinal fluid, culture media, lung lavage, plasma, serum, feces, saliva, semen, sweat, tears, urine, and vaginal secretion. In embodiments, the solid sample is selected from one or more of a biopsy, a fixed sample, a hair sample, a fingernail sample, and a toenail sample. In embodiments, the biological sample is part of or is a swab sample, a scrape sample, a skin sample, or an oral sample.

In any of the above aspects, the sample is collected from a surface of a medical device. In embodiments, the medical device is a catheter. In any of the above aspects, the sample contains a biofilm.

In any of the above aspects, the set of genomes contains at least 2 genomes. In any of the above aspects, the set of genomes contains at least about 5 genomes. In any of the above aspects, the set of genomes contains at least about 500 genomes.

In any of the above aspects, the set of genomes are derived from a plurality of strains of a species. In any of the above aspects, the set of genomes are derived from a set of species derived from a common family. In any of the above aspects, the organisms include a eukaryote or a prokaryote. In embodiments, the eukaryote is a fungus. In embodiments, the prokaryote is a bacterium or archaeon.

In any of the above aspects, the organisms include a pathogen. In embodiments, the pathogen is selected from one or more of Aerobacter, Aeromonas, Acinetobacter, Actinomyces israelii, Agrobacterium, Bacillus, Bacillus anthracis, Bacteroides, Bartonella, Bordetella, Bortella, Borrelia, Brucella, Burkholderia, Calymmatobacterium, Campylobacter, Citrobacter, Clostridium, Corynebacterium, Enterobacter, Enterococcus, Erysipelothrix rhusiopathiae, Escherichia, Faecalibacterium, Francisella, Fusobacterium nucleatum, Gardnerella, Haemophilus, Hafnia, Helicobacter, Klebsiella, Legionella, Leptospira, Listeria, Morganella, Moraxella, Mycobacterium, Neisseria, Pasteurella, Proteus, Providencia, Pseudomonas, Rickettsia, Salmonella, Serratia, Shigella, Staphylococcus, Stentorophomonas, Streptococcus, Treponema, Xanthomonas, Vibrio, and Yersinia. spp.

In any of the above aspects, the organisms include a species belonging to the genus Akkermansia and/or Bifidobacterium. In any of the above aspects, the organisms include a strain of E. coli selected from one or more of AIEC, DAEC, EAEC, EHEC, EIEC/Shigella, EPEC, ETEC, ExPEC, NMEC, SEPEC, ST131, and UPEC. In any of the above aspects, the set of probes specifically target a pathogenic strain(s) of E. coli.

In any of the above aspects, the organisms make up less than about 5% of the complex biological sample by relative abundance. In any of the above aspects, the organisms make up less than about 1% of the complex biological sample by relative abundance.

In any of the above aspects, the polynucleotides are enriched by a factor of at least about 2. In any of the above aspects, the polynucleotides are enriched by a factor of at least about 5.

In any of the above aspects, the polynucleotide-probe complexes contain a non-biased representation of sequence diversity in the complex biological sample.

In any of the above aspects, the probes contain DNA, RNA, or modified nucleobases.

In any of the above aspects, each probe within the set contains from about 50 to about 200 nucleotides.

In any of the above aspects, each probe shares at least about 90% nucleotide sequence identity across the length thereof with a gene sequence in at least one of the clusters.

In any of the above aspects, the clusters contain all genes or a subset of genes of the organisms. In any of the above aspects, the subset of genes of the organisms contains, or only contains, those genes associated with a function(s) of interest. In embodiments, the function(s) of interest includes antibiotic resistance and/or pathogenicity. In any of the above aspects, the subset of the set of probes contains probes targeting genes associated with a function(s) of interest. In any of the above aspects, the set of probes contains, or only contains, those genes targeting gene sequences belonging to a subset of the clusters.

In any of the above aspects, the set of probes or subset thereof contains at least about 1,000 probe sequences. In any of the above aspects, the set of probes or subset thereof contains at least about 10,000 probe sequences. In any of the above aspects, the set of probes or subset thereof contains at least about 50,000 probe sequences. In any of the above aspects, the set of probes or subset thereof contains between 100,000 to 2,000,000 probe sequences. 100. The set of probes of claim 98 or claim 99, where the set of probes contains about or at least about 50,000 sequences. In any of the above aspects, the set of probes contains about or at least about 500,000 sequences. In any of the above aspects, the set of probes contains about or at least about 1,000,000 sequences. In any of the above aspects, the set of probes contains about or at least about 2,500,000 sequences.

In any of the above aspects, each probe contains a binding member. In any of the above aspects, the binding member is streptavidin, biotin, an antigen-binding molecule, or an antigen. In any of the above aspects, the binding member is biotin. In any of the above aspects, the probes collectively contain a plurality of distinct binding members.

In any of the above aspects, the solid support contains metal, glass, or a polymeric material. In any of the above aspects, the solid support contains beads. In any of the above aspects, the solid support is a biochip.

In any of the above aspects, each probe is coupled to the solid support by a direct covalent linkage or by a bridge containing a binding member bound by a capture molecule. In any of the above aspects, the capture molecule is streptavidin, biotin, an antigen-binding molecule, or an antigen. In any of the above aspects, the capture molecule is streptavidin. In any of the above aspects, the solid support contains a plurality of capture molecules.

In any of the above aspects, the polynucleotides contain RNA or DNA. In any of the above aspects, the polynucleotides contain mRNA. In any of the above aspects, the polynucleotides contain genomic DNA.

In any of the above aspects, the polynucleotides contain at least part of or are a polynucleotide sequence library. In embodiments, the polynucleotide sequence library contains cDNA or mRNA. In embodiments, the polynucleotide sequence library contains genomic DNA.

In any of the above aspects, the method involves an amplification step. In any of the above aspects, the polynucleotides are unamplified.

In any of the above aspects, the gene sequences within each cluster share at least about 50% nucleotide sequence identity. In any of the above aspects, the gene sequences within each cluster share at least about 60% nucleotide sequence identity. In any of the above aspects, the gene sequences within each cluster share at least about 70% nucleotide sequence identity. In any of the above aspects, the gene sequences within each cluster share at least about 80% nucleotide sequence identity.

In any of the above aspects, the clusters are clusters of homologous gene sequences. In any of the above aspects, the clusters are orthogroup clusters. In embodiments, the orthogroup clusters are identified based upon gene synteny.

In any of the above aspects, the method further involves excluding from the set of probes those probes with greater than about 80% nucleotide sequence identity to one or more reference sequences. In any of the above aspects, the method further involves excluding from the set of probes those probes having 25 or more contiguous nucleotides that are identical to one or more reference sequences. In embodiments, the reference sequences contain genome sequences. In embodiments, the reference sequences contain genome sequences for bacteria selected from one or more of Citrobacter freundii, Salmonella enterica, and Klebsiella pneumoniae. In embodiments, the reference sequences contain gene sequences derived from Bacteroidetes and/or Firmicutes.

In any of the above aspects, the probes collectively cover at least 10% of the gene sequences within each cluster. In any of the above aspects, the probes collectively cover at least 50% of the gene sequences within each cluster. In any of the above aspects, the probes collectively cover at least 75% of the gene sequences within each cluster. In any of the above aspects, the probes collectively cover at least 90% of the gene sequences within each cluster. In embodiments, coverage of each probe is determined using a cover extension of at least about 20 bp. In embodiments, coverage of each probe is determined using a cover extension of at least about 50 bp.

In any of the above aspects, the probes collectively target at least 50% of all gene sequences in each of the gene clusters. In any of the above aspects, the probes collectively target at least 75% of all gene sequences in each of the gene clusters. In any of the above aspects, the probes collectively target at least 95% of all gene sequences in each of the gene clusters. In any of the above aspects, the set of probes enrich sequences derived from the organisms without significant bias.

In any of the above aspects, the probes capture intergenic regions of the set of genomes.

In any of the above aspects, the method further involves sequencing the polynucleotides of the polynucleotide/probe complexes.

In any of the above aspects, the complex biological sample is derived from a subject. In embodiments, the subject is an animal. In embodiments, the animal is a mammal. In embodiments, the mammal is a human. In embodiments, the subject has or has had an infection associated with the organisms. In embodiments, the infection is a chronic or recurring infection.

In any of the above aspects, each probe contains a unique molecular identifier. In any of the above aspects, each probe contains a bar code. In any of the above aspects, each probe contains a detectable moiety. In any of the above aspects, each probe contains a binding member.

The invention provides compositions and methods that are useful in the design and use of probes for characterizing organisms present in a complex biological sample. Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “pan-genomic” or “pan-genome” is meant a set of genomic sequences from two or more organisms or strains. The genomic sequences can be gene sequences. The organisms can be strains of a species of microorganism. The set of genomic sequences can contain all or a subset of genes encoded by the organisms. The set of genomic sequences can include gene sequences from about or at least about 1, 2, 3, 4, 5, 10, 25, 50, 75, 100, 250, 500, 1000, 1100, 1200, 1300, 1400, 1500, 2000, 5000, or 10000 species. The gene sequences can represent a set of 1%, 5%, 10%, 20%, 30%, 40%, 50%, 60%, 70%, 80%, 90%, 99%, or 100% of all genes encoded by the organisms. The set of genomic sequences can include genomic sequences from a set or subset of known strains of a species of microorganism. The gene sequences may contain introns and/or exons.

By “pan-transcriptomic” or “pan-transcriptome” is meant a set of sequences corresponding to mRNA transcribed from a pan-genome. The sequences corresponding to mRNA can be DNA sequences (e.g., cDNA) or RNA sequences. The sequences corresponding to mRNA can be sequences of mRNA transcripts transcribed from a set of genomic sequences from a pan-genome.

By “complex biological sample” is meant a biological sample containing polynucleotides derived from two or more organisms. In embodiments, a complex biological sample contains polynucleotides derived from a first organism (e.g., microorganism, such as a prokaryote, eukaryote), and polynucleotides derived from a second organism (e.g., a host, a subject, a tumor sample, a biopsy, a organism disposed in an environmental sample, and the like) with which the first organism is associated. A non-limiting example of a complex biological sample is a microbiome, optionally associated with a host organism (e.g., disposed within or on a subject). A further non-limiting example of a complex biological sample is a sample comprising a microbe (e.g., a fungus or a prokaryotic organism) and cells from a second organism (e.g., an animal, a mammal, a human, and the like). In embodiments, a complex biological sample contains polynucleotides derived from about or at least about 2, 3, 4, 5, 6, 7, 8, 9, 10, 11, 12, 13, 14, 15, 25, 50, 100, 200, 300, 400, 500, 1000, 2,000, 3,000, 4,000, 5,000, 6,000, 7,000, 8,000, 9,000, or 10,000 organisms. In embodiments, a complex biological sample contains polynucleotides derived from no more than about 2, 3, 4, 5, 6, 7, 8, 9, 10, 11, 12, 13, 14, 15, 25, 50, 100, 200, 300, 400, 500, 1000, 2,000, 3,000, 4,000, 5,000, 6,000, 7,000, 8,000, 9,000, or 10,000 organisms. A further non-limiting example of a complex biological sample is an artificial or natural microbial community comprising two or more microbes, and/or an artificial or natural association or organisms (e.g., in/on an organoid or in/on a plant). In some embodiments, the complex biological sample is a soil sample, water sample, or other environmental sample.

By “microbiome” is meant the microbial community associated with a sample. The microbiome can be complex, where “complex” can mean that the microbiome contains more than one species of organism. The microbiome can contain, for example, prokaryotes (e.g., bacteria, archaea), and/or eukaryotes (e.g., protists, fungi, yeast).

By “orthogroup cluster” is meant a set of genomic sequences predicted to be evolutionarily derived from a common ancestor. Orthogroup clusters can be determined based upon sequence data using an algorithm, a non-limiting example of which is SynerClust (Georgescu, Christophe H., Abigail L. Manson, Alexander D. Griggs, Christopher A. Desjardins, Alejandro Pironti, Ilan Wapinski, Thomas Abeel, Brian J. Haas, and Ashlee M. Earl. 2018. “SynerClust: A Highly Scalable, Synteny-Aware Orthologue Clustering Tool.” Microbial Genomics. doi.org/10.1099/mgen.0.000231). An ortholog cluster can include a set of genes descended from a single gene from a common ancestor of a set of species of interest.

The term “species” has its conventional meaning. In embodiments, a “species” of an organism share about or at least about 60%, 65%, 70%, 75%, 80%, 90%, 95%, 97%, or 99% nucleotide sequence identity across a set of genes encoded by the organisms, where the set of genes can include all or at least about 50%, 60%, 70%, 80%, 90%, 95%, or 99% of all genes encoded by each organism. In embodiments, a “species” of an organism share about or at least about 60%, 65%, 70%, 75%, 80%, 90%, 95%, 97%, or 99% nucleotide sequence identity for a specified gene sequence or a fragment thereof. The specified gene sequence can be a 16S sequence. In embodiments, a species includes those organisms encoding the specified gene sequence (e.g., the 16S gene sequence) where each specified sequence encoded by an organism within the species shares at least 97% nucleotide sequence identity. In embodiments, species of an organism share an average nucleotide identity (ANI) of about or at least about 90%, 95%, 96%, 97%, 98%, 99%, or higher. ANI can be determined using various methods, including, as non-limiting examples, those described in Jain, et al. “High throughput ANI analysis of 90K prokaryotic genomes reveals clear species boundaries” Nature Communications, vol. 9, article no. 5114 (2018), the entire disclosure of which is incorporated herein by reference in its entirety for all purposes.

By “strains of an organism” or “strains of a species” is meant an organism belonging to a species and that is phenotypically or genetically distinguishable from other members of the species. A strain may be distinguishable from other members of a species based upon an altered genomic sequence and/or based upon the presence or absence of a genomic sequence. A strain may be distinguishable from other members of a species based upon sequences encoded by the organism, where the sequences may be contained within a plasmid or other gene transfer vector.

By “capture molecule” is meant any polypeptide, polynucleotide, or fragment or analog thereof capable of specifically binding an analyte of interest. In some embodiments, the capture molecule is coupled to a solid support.

By “agent” is meant any chemical compound or functional group, antibody, nucleic acid molecule, or polypeptide, or fragments thereof.

By “alteration” is meant an increase or a decrease in the structure, expression levels, or activity of a gene or polypeptide as detected by standard art known methods such as those described herein. As used herein, an alteration includes a change of about or of at least about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99%, or 100%. In some embodiments, the alteration is in the relative abundance of a microbe in a complex biological sample (e.g., a microbiome).

By “analog” is meant a molecule that is not identical, but has analogous functional or structural features.

By “binding member” is meant an agent capable of being bound by a capture molecule. In some embodiments, a binding member is avidin or streptavidin. In other embodiments, an agent is an antibody or a polypeptide bound by the antibody.

“Microarray” means a collection of nucleic acid molecules or polypeptides from one or more organisms arranged on a solid support (for example, a chip, plate, or bead). These nucleic acid molecules or polypeptides may be arranged in a grid where the location of each nucleic acid molecule or polypeptide remains fixed to aid in identification of the individual nucleic acid molecules or polypeptides. A microarray may include, for example, nucleic acid molecules representing all, or a subset, of the open reading frames of an organism, or of the polypeptides that those open reading frames encode. In one embodiment, the nucleic acid molecules of the array are defined as having a common region of the genome having limited homology to other regions of an organism's genome. A microarray may also be enriched for a particular type of gene. In some embodiments the microarray is a biochip comprising DNA or RNA, in which case the biochip may be referred to as a DNA microarray chip or an RNA microarray chip, respectively.

By “NanoString assay” is meant a method involving contacting a sample containing nucleotide molecules with a plurality of labeled target-specific nucleic acid probes for the detection and/or quantification of targeted nucleotide sequences in the sample. In embodiments, a NanoString assay is carried out according to methods described in International Patent Application Publication Nos. WO2003003810. In embodiments, the NanoString assay is used as a non-solid based approach alternative to a microarray-based method.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

By “consist essentially” it is meant that the ingredients include only the listed components along with the normal impurities present in commercial materials and with any other additives present at levels which do not affect the operation of the disclosure, for instance at levels less than 5% by weight or less than 1% or even 0.5% by weight.

By “cover” is meant a region of a target sequence to which a probe is predicted to bind. The probe covers a target sequence over that portion of the target sequence with which the probe shares about or at least about 80%, 90%, 95%, 99%, or 100% nucleotide sequence identity over the full length of the capture probe or over a contiguous portion of the capture probe about or at least about 10, 20, 30, 40, 50, 60, 70, 80, 90, 100, 125, 150, 175, or 200 base pairs in length or the full length of the capture probe.

By “cover extension” is meant an artificial extension of a region of a target sequence considered as being covered by a probe. The cover extension can be an extension at the 3′ and/or 5′ end of the covered region. The cover extension can be about or at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 55, 60, 75, 80, 85, 90, 95, or 100 base pairs in length. In embodiments, the cover extension is no more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 55, 60, 75, 80, 85, 90, 95, or 100 base pairs in length.

“Detect” refers to identifying the presence, absence or amount of the analyte to be detected. The analyte can be a genomic sequence and/or a sequence corresponding to an mRNA molecule or gene sequence.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include urinary tract infections, Crohn's disease, traveler's diarrhea, E. coli 0157: H7 infection, neonatal meningitis (NMEC), sepsis, a lung infection, a periodontal disease, bacterial vaginosis, H. pylori gastritis, irritable bowel disease (IBD), mastitis, and cirrhosis. In some instances, the disease is associated with a pathogen (e.g., a pathogenic microbe). In some embodiments, the disease is a cancer. Some prokaryotes are tumorigenic and, therefore, are associated with development of a tumor or cancer in a subject. The disease can be chronic or recurring. A chronic infection can be an infection that recurs about or at least about 3, 4, 5, 6, 7, 8, 9, 10, 11, 12, 13, 14, or 15 times in a subject and/or that persists and/or recurs over a period of about or at least about 5 days, 6 days, 7 days, 1 month, 2 months, 3 months, 4 months, 5 months, 6 months, 7 months, 8 months, 9 months, 10 months, 11 months, or 12 months.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “homologous” is meant sequences sharing sequence identity. In embodiments, homologous gene sequences share about or at least about 20%, 30%, 40%, 50%, 60%, 70%, 80%, 90%, 95%, or 99% nucleotide sequence identity.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” nucleic acid molecule is sufficiently free of other materials such that any impurities do not materially affect the biological properties of the nucleic acid molecule or cause other adverse consequences (e.g., interfere with a PCR reaction). That is, a nucleic acid molecule of this invention is purified if it is substantially free of cellular material, viral material, or other impurities. Purity and homogeneity are typically determined using analytical chemistry techniques, for example, polyacrylamide gel electrophoresis or high performance liquid chromatography.

By “molecular identifier” or “unique molecular identifier” is meant an agent coupled to a molecule that can be used to identify the molecule.

As used herein, “obtaining” as in “obtaining an agent” includes synthesizing, purchasing, or otherwise acquiring the agent. In embodiments, the agent is an oligonucleotide or a set of oligonucleotides (e.g., a probe set).

By “reference” is meant a standard or control condition. The reference can be a reference genome or a set of reference genomes. The reference can include a full genome sequence or set of genome sequences, or a set of genes encoded by a genome sequence or set of genome sequences.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant an animal. The animal can be a mammal. The mammal can be a human or non-human mammal, such as a bovine, equine, canine, ovine, rodent, or feline.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1D provide schematic illustrations of a hybrid capture process used for enriching nucleic acids from minor members of a complex community. In brief, the process involved designing a probe set to cover genomes of interest (FIG. 1A), mixing the probe set with a meta'omic (e.g., metatranscriptomic or metagenomic) library to allow for hybridization to complementary sequences (FIG. 1B), binding probes (and hybridized sequences) to capture beads (FIG. 1C), and eluting bound library molecules off of the beads for downstream processing (e.g., PCR amplification and/or sequencing) (FIG. 1D). FIG. 1A is a schematic showing construct sequencing libraries containing a small number of reads from a low-abundance target organism (dark shading) in a metagenomic background (light shading). FIG. 1B shows short oligonucleotide probes representing a bait set attached to biotin (circles) hybridized with complementary library molecules. FIG. 1C shows a sequencing library, biotinylated probes, and capture beads incubated overnight. FIG. 1D shows bound library molecules eluted off the beads. The eluted molecules were further amplified by PCR (embodiments without amplification are contemplated), and then sequenced. This process resulted in a final sequencing library highly enriched for species of interest, ready for sequencing. In FIGS. 1A-1D, biotin is represented by a circle and streptavidin is represented as an open semi-circle.

FIG. 2 provides a flow chart showing a process used for E. coli pan-genome probe design. The process involved steps a) to g). In step a), all 295 available RefSeq complete E. coli and Shigella genomes and 3,141 additional genomes from the NCBI Pathogens database were downloaded from June to August 2017. In step b), genomes were clustered using a k-mer based algorithm with a threshold of a 95% Jaccard similarity to select a set of non-redundant references for downstream analysis. In step c), these genomes were uniformly annotated with the Broad Institute's prokaryotic genome annotation pipeline (Schreiber, Henry L., 4th, Matt S. Conover, Wen-Chi Chou, Michael E. Hibbing, Abigail L. Manson, Karen W. Dodson, Thomas J. Hannan, et al. 2017. “Bacterial Virulence Phenotypes of Escherichia Coli and Host Susceptibility Determine Risk for Urinary Tract Infections.” Science Translational Medicine 9 (382). doi.org/10.1126/scitranslmed.aaf1283) and then orthogroups were constructed with SynerClust (Georgescu, Christophe H., Abigail L. Manson, Alexander D. Griggs, Christopher A. Desjardins, Alejandro Pironti, Ilan Wapinski, Thomas Abeel, Brian J. Haas, and Ashlee M. Earl. 2018. “SynerClust: A Highly Scalable, Synteny-Aware Orthologue Clustering Tool.” Microbial Genomics. doi.org/10.1099/mgen.0.000231). In step d), orthogroups were filtered to remove rare orthogroups, resulting in 64,580 orthogroups comprising 8, 168,837 genes. In step e), the genes from each remaining orthogroup were then clustered at >80% nucleotide sequence identity with UCLUST (Edgar, Robert C. 2010. “Search and Clustering Orders of Magnitude Faster than BLAST.” Bioinformatics 26 (19): 2460-61) to reduce computational runtime. In step f), These UCLUST clusters were each used as input into CATCH (Metsky, Hayden C., Viral Hemorrhagic Fever Consortium, Katherine J. Siddle, Adrianne Gladden-Young, James Qu, David K. Yang, Patrick Brehio, et al. 2019. “Capturing Sequence Diversity in Metagenomes with Comprehensive and Scalable Probe Design.” Nature Biotechnology. doi.org/10.1038/s41587-018-0006-x) to generate probe sequences. In step g), probes were filtered to remove universal probes, as well as probes that target genomes of major members of the human gut microbiome from the Bacteroidetes and Firmicutes phyla, retaining a total of 892,415 probes for synthesis, which are provided in the Sequence Listing as SEQ ID NOs: 1 to 892415.

FIGS. 3A-3D provide stacked bar graphs and plots demonstrating that hybrid capture dramatically enriched E. coli DNA without bias from a mock community of known composition, and provided greatly increased sequence information for each of the four component E. coli strains. FIG. 3A is a stacked bar graph presenting results from hybrid capture (HC) performed on sequencing libraries from a mock community consisting of a total of 1% DNA from four known strains of E. coli in a background of 99% human DNA. The four strains represented different phylogenetic groups in an unequal mixture (80:15:4.9:0.1). The total relative abundances of E. coli sequence, as well as the relative proportions of each strain (four colors) are shown. FIG. 3B is a plot presenting depth of coverage pre- and post-HC for each of the four indicated strains. The probe set successfully enriched the mock community of the four diverse E. coli strains. The relative abundance of the E. coli strains was increased by about 40-fold, on average, and there was no change in strain ratios. Therefore, depth of coverage of diverse E. coli strains was enriched using hybrid capture in a non-biased manner. FIG. 3C provides a collection of plots presenting genome coverage pre- and post-HC for each of the four strains. Thin lines represent pre-HC data, while bold lines represent post-HC data. The dashed vertical line represents 5× coverage, a minimum cutoff for depth. Over 70% of all strains genomes was sequenced at a coverage of 5× or greater. FIG. 3D provides a set of four plots showing average depth of coverage for positions along the reference genome, in relation to the closest predicted probe binding site(s), for each of the four indicated strains. “2+ probes” indicates regions where two or more probes are predicted to bind, while “1+ probes” indicates regions in which at least one probe is predicted to bind. Error bars denote standard deviation. Numbers above error bars indicate the number of positions across the genome considered for each genome for each X axis category. Thin lines represent pre-HC data, while bold lines represent post-HC data.

FIGS. 4A and 4B present a plot and a collection of stacked bar graphs demonstrating that hybrid capture (HC) enriched E. coli from human stool samples and revealed previously missed strains. FIG. 4A is a plot showing pre- and post-HC relative abundance of detected E. coli strains from clinical samples. Strain rank indicates most (1) to least (3) abundant strains in the sample. Dots to the left the dashed vertical line indicate novel strains found exclusively in post-HC data. n.d.=not detected. FIG. 4B provides a set of stacked bar graphs showing detected strains of E. coli in pre- and post-HC samples using StrainGE (Dijk, Lucas R. van, Bruce J. Walker, Timothy J. Straub, Colin J. Worby, Alexandra Grote, Henry L. Schreiber, Christine Anyansi, et al. 2021. “StrainGE: A Toolkit to Track and Characterize Low-Abundance Strains in Complex Microbial Communities.” Cold Spring Harbor Laboratory. doi.org/10.1101/2021.02.14.431013) from clinical samples. Samples marked with a black asterisk show instances in which HC detects additional strain(s) in the given sample. The three samples marked with † indicate the samples used for a single-strain metatranscriptomic analysis.

FIGS. 5A and 5B present a collection of plots showing that hybrid capture (HC) enriched E. coli transcripts from clinical samples. Pre-HC vs. post-HC expression levels are presented in counts per million (CPM) per detectable transcript (minimum 10 CPM). Each point represents one or more unique transcripts, where the shading represents different E. coli strains and the size represents the number of unique transcripts (i.e., genes) at that given expression value level. Points left of the vertical dashed line represent transcripts undetected pre-HC, while points below the dashed horizontal line represent transcripts undetected post-HC. Points below the diagonal line had higher normalized expression levels pre-HC, while points above the line had higher normalized expression levels post-HC. The number of unique transcripts in each quadrant are indicated. FIG. 5A provides a collection of plots presenting single-strain samples (strains indicated above each respective plot) where reads were aligned (minimum MQ of 5) to the detected strain's reference. FIG. 5B presents a set of plots showing all samples where reads were aligned (minimum MQ of 5) to each participant's concatenated strains' genomes. Due to mapping quality filtering, reads that aligned equally well to all reference genomes within the same participant, and therefore the core E. coli genome, were excluded from analysis.

FIG. 6 presents a schematic providing details relating to a probe design procedure.

FIG. 7 presents a set of histograms demonstrating that very few regions of each strain's reference genome were distant from predicted probe binding sites. X axis is the distance between predicted probe binding sites (in kilobases). Y axis is the number of regions at a given distance. Y axis has been truncated to zoom in on the fewer larger distances, as there were thousands of instances where probes were predicted to bind within 10 bases of each other.

FIGS. 8A-8D each provide box-and-whisker plots showing stool sample library sequence quality pre-vs. post-hybrid capture (HC). FIG. 8A presents box-and-whisker plots showing percent of reads for each stool sample with a given mean sequence quality (PHRED score) for pre- and post-HC data. FIG. 8B presents box-and-whisker plots showing base quality (PHRED score) distributions along reads for pre- and post-HC data. FIG. 8C presents box-and-whisker plots showing percent of adapter content along reads for pre- and post-HC data. Adapter content was directly related to the fragment insert size. Smaller insert sizes lead to more adapter content in the read. FIG. 8D presents box-and-whisker plots showing percent of sequences at varying duplication levels (1=unique sequence) for pre- and post-HC data.

FIG. 9 is a plot showing post-HC data had high depth and breadth of coverage. Depth of coverage (X) vs. breadth of coverage (%) for all human stool samples, based on StrainGST estimates. Shading indicates the most (darkest shade) to least (lightest shade) abundant strain. In the figure, the most abundant strain ranks cluster in the upper-right portion of the plot, as indicated by the arrow.

FIGS. 10A and 10B present box-and-whisker plots and a principal coordinate analysis plot demonstrating that non-E. coli community profile was unaltered by HC. FIG. 10A provides box-and-whisker plots showing Bray-Curtis Dissimilarity distributions for various pairwise sample comparisons, comparing the taxonomic profile at the species level for non-E. coli (and Shigella) species. “Different individual” comparisons compare two samples from different study participants. “Same individual” comparisons compare two samples from the same study participant, but different time points. “Same sample” comparisons compare pre-HC to post-HC of the same sample. “Pre-HC” alone values are comparisons of two samples, both pre-HC. “Post-HC alone” values are comparisons of two samples, both post-HC. “Pre-vs post-HC” values are comparisons where one sample was pre-HC and the other was post-HC. FIG. 10B is a plot presenting a Principal Coordinate Analysis (PCoA) using the Bray-Curtis distances. The first two principal coordinates are shown. Each point connected by a line represents a pair of samples, pre-HC (circle) and post-HC (triangle). Each shade and number represents a study participant (subject).

FIG. 11 provides a schematic illustrating an approach to the design of pan-genome probes for the capture of full E. coli pan-genome diversity. The probes were designed based on 3,436 E. coli and Shigella reference genomes spanning pan-genome diversity.

FIGS. 12A-12C provide schematics showing the fim operon promoter. FIG. 12A provides a schematic indicating that the fim operon promoter (fimS) is invertible, and that the inversion is mediated by site-specific recombinases encoded upstream of the operon. FIG. 12B provides a schematic showing how sequence alignments can be used to determine the orientation (e.g., on or off) of fimS. FIG. 12C provides a schematic showing fimS phase variation between the on and off configurations as well as the positioning of informative and non-informative sequence alignments (see also FIG. 12B).

FIG. 13 provides a plot showing a correlation between read and probe coverage of the fimS region. Despite gen-centric design, probes still enriched the boundary region of fimS.

FIG. 14 provides a plot showing that fimS detection was dependent on sample E. coli content.

FIG. 15 provides box-and-whisker plots showing that hybrid capture provided 10× enrichment of both inversion phases (i.e., on and off) of the fimS promoter. Hybrid capture samples were compared with pre-hybrid capture samples collected at the same time point and sequenced to the same depth.

FIG. 16 provides a plot showing the % fimS “on” status (y-axis) for samples from participants (x-axis) in the Urinary Tract Infection Microbiome (UMB) Project.

FIG. 17 provides a collection of box-and-whisker plots showing the number of reads aligned to the indicated genes (x-axis: fimB, fimE, fimA, fimI, fimC, fimD, fimF, fimG, fimH) for sequenced RNA hybrid-capture samples prepared for stool samples from participants in the UMB Project.

FIG. 18 provides a plot showing a comparison between the predicted (model) and observed correlation between operon expression and fimS being in the on orientation. “CI” indicates “confidence interval.” The y-axis represents the total expression of the protein-coding region of the fim operon (fimA-fimH, which includes fimI).

FIG. 19 provides a schematic overview of the experimental design for the Urinary Tract Infection Microbiome (UMB) Project. The control group included 16 healthy women having only had 1 or less urinary tract infections previously. The recurrent urinary tract infection (rUTI) group included 15 women having had at least 3 urinary tract infections over the year prior to participating in the UMB Project.

FIG. 20 provides stacked bar graphs showing the relative abundance for RNA (top) and DNA (bottom) of fim reads per strain per sample from multi-strain samples. In FIG. 20, the stars indicate samples where the minor strain expressed fim at a greater level than the major strain.

### DETAILED DESCRIPTION OF THE INVENTION

The invention features compositions and methods useful for characterizing complex biological samples (e.g., microbiomes) comprising a variety of organisms and/or strains of organisms.

The present invention is based, at least in part, upon the discovery of a hybrid capture strategy involving the use of a custom probe set that specifically and sensitively characterizes diverse E. coli strains in complex metagenomic samples. As described in the examples, a computational approach was taken to design probes for characterizing E. coli present in a complex biological sample. As described in detail below, this approach identified a set of approximately 900,000 oligonucleotide probes that hybridize to the E. coli pan-genome, represented by over 1,700 E. coli reference genomes, encoding more than 8 million genes and encompassing the vast majority of known diversity for the species. This probe set was used to successfully enrich E. coli from a mock metagenomic community consisting of four diverse, known strains of E. coli in a background of 99% human reads, as well as in metagenomic and metatranscriptomic sequencing libraries derived from human stool samples. Hybrid capture was unbiased and enriched E. coli sequence from DNA libraries by an average of approximately 40-fold, and from RNA libraries by approximately 23-fold. This enrichment allowed for observation of multiple E. coli strains within a sample with a high depth and breadth of coverage, detection of additional low abundance E. coli strains, and observation of over one thousand unique E. coli transcripts that could not be detected before hybrid capture. Thus, in embodiments, the methods and probes of the present invention are used to investigate native in vivo biology of diverse E. coli or other microbial species of interest in the gut and other environments, where E. coli or the other microbial species of interest is a minor component of the microbial community using substantially unbiased, culture-free metagenomic and/or metatranscriptomic shotgun sequencing. The methods provided herein can also be generally applied to other highly diverse, but low abundance bacteria; for example, Enterococcus spp. (Enterococcus faecalis, Enterococcus faecium), Ruminococcus spp. (e.g., R. gnavus), probiotic organisms such as Bifidobacterium spp. or Lactobacillus spp., Staphylococcus spp., Akkermansia spp., and Faecalibacterium spp.

### Hybrid capture (HC) probes

### Design

The disclosure provides methods to design probe sets using a pan-genome approach to characterize complex biological samples comprising highly diverse bacterial species, or a limited number (e.g., 1, 2, 3, 4, 5, or more) of bacterial species associated with a sample (e.g., a sample derived from a host organism). Using a set of reference genes derived from a known pan-genome, probes are designed to selectively and sensitively enrich for genes without strain or reference bias. In embodiments, the reference genes are related by function and/or sequence. Advantageously, such probes are synthesized and conjugated with a capture molecule, such as biotin, allowing for the low-cost, at-scale enrichment of sequences from complex biological samples (e.g., complex microbiome metagenomes). Various methods described in U.S. Patent Application Publications 2019/0330706, 2019/019766, and/or 2018/0340215, which are incorporated herein in their entirety, are suitable for use in the methods of the present invention.

The present invention features methods for generating probes for use in characterizing a complex biological sample (e.g., a microbiome). In some embodiments, the design method involves (a) constructing candidate probes targeting clusters of gene sequences, where the candidate probes collectively have a hybridization pattern for the clusters of gene sequences; (b) determining an individual hybridization pattern for each candidate probe within each gene cluster to provide a collection of individual hybridization patterns; and (c) subjecting the individual hybridization patterns to a set cover solving process to reduce the number of candidate probes to provide a set of selected probes targeting genes within each gene cluster. A set cover solving process is a process by which a minimum number of probes are selected to target sequences corresponding to a set of target genes. A sequence region considered as targeted by a probe can be extended at the 3′ and/or 5′ end by using a cover extension.

In some embodiments, the gene sequences are derived from a plurality of strains of an organism present in a complex biological sample (e.g., a microbiome). The organisms can be members of the same or of different species. The gene sequences can be derived from one or more organisms present in a complex biological sample (e.g., a microbiome). The gene sequences can be derived from a representative set of genera that are phylogenetically classified as belonging to a common family. The sequences can be derived from genomes of a set of organisms. The sequences can be derived from about or at least about 2, 3, 4, 5, 10, 15, 20, 25, 50, 100, 150, 200, 250, 500, 1000, 1100, 1200, 1300, 1400, 1500, 1600, 1700, 1800, 1900, 2000, 3000, 4000, 5000, 10000, 15000 or more genomes. The sequences can be derived from one or more of the 3,141 GenBank genomes and/or the 295 RefSeq genomes listed in the Examples.

Genes sequences within each cluster of gene sequences share a level of nucleotide sequence identity. The level of nucleotide sequence identity within the cluster can be about or at least about 15%, 20%, 25%, 30%, 35%, 40%, 45%, 50%, 55%, 60%, 65%, 70%, 75%, 80%, 85%, 90%, or 95%.

Probes useful in the methods described herein target at least about 5%, 10%, 15%, 20%, 25%, 50%, 75%, 80%, 85%, 90%, 99%, or 100% of all gene sequences contained within all or a sub-set of the clusters of gene sequences. The probes cover about or at least about 5%, 10%, 15%, 20%, 25%, 50%, 75%, 80%, 85%, 90%, 99%, or 100% of all gene sequences contained within all or a subset of the clusters of gene sequences.

The subset of clusters of gene sequences are selected in various embodiments to comprise sequences derived from genes associated with a function(s) of interest. Thus, a set of probes can be selected to target a set of genes related by sequence and/or function. The function of interest can be, as non-limiting examples, antibiotic resistance and/or pathogenicity. Therefore, the present invention provides for probe sets targeting a gene(s) across a pan-genome or pan-transcriptome associated with a function(s) of interest. Such probe sets can be designed for use in methods to target sequences from one or more organisms in a non-biased manner.

The set of gene sequences can contain or consist of sequences useful in identification of an organism of interest, including specific strains thereof. In embodiments, the sequences useful in identification of the organism of interest are sequence-tagged sites. A sequence tagged site is a short (e.g., 100 to 500, or 200 to 500 base pair) DNA or RNA sequence that has a single occurrence in the genome of an organism and whose location and base sequence are known.

Virtually any organism can be characterized using the methods described herein. In some embodiments, the organisms include pathogenic bacteria, including gram positive and gram negative bacteria. The pathogenic bacteria can be opportunistic pathogens. Exemplary pathogens include, but are not limited to, microbes selected from Aerobacter, Aeromonas, Acinetobacter, Actinomyces israelii, Agrobacterium, Bacillus, Bacillus anthracis, Bacteroides, Bartonella, Bordetella, Bortella, Borrelia (e.g., Borrelia burgdorferi), Brucella, Burkholderia, Calymmatobacterium, Campylobacter, Citrobacter, Clostridium (e.g., Clostridium perfringens, Clostridium tetani), Corynebacterium (e.g., Corynebacterium diphtheriae), Enterobacter (e.g., Enterobacter aerogenes), Enterococcus (e.g., Enterococcus faecalis, Enterococcus faecium), Erysipelothrix rhusiopathiae, Escherichia, Faecalibacterium, Francisella, Fusobacterium nucleatum, Gardnerella, Haemophilus (e.g., Haemophilus influenzae), Hafnia, Helicobacter (e.g., Helicobacter pylori), Klebsiella (e.g., Klebsiella pneumoniae), Legionella, Leptospira, Listeria (e.g., Listeria monocytogenes), Morganella, Moraxella, Mycobacterium (e.g., M. avium, M. gordonae, M. intracellulare, M. kansasii, M. tuberculosis), Neisseria (e.g., Neisseria gonorrhoeae, Neisseria meningitidis), Pasteurella (e.g., Pasturella multocida), Proteus, Providencia, Pseudomonas, Rickettsia, Salmonella, Serratia, Shigella, Staphylococcus (e.g., Staphylococcus aureus), Stentorophomonas, Streptococcus (e.g., Streptobacillus moniliformis, Streptococcus agalactiae, Streptococcus bovis, Streptococcus faecalis, Streptococcus pneumoniae, and Streptococcus pyogenes), Treponema (e.g., Treponema pallidum, Treponema pertenue), Xanthomonas, Vibrio, and Yersinia. Exemplary strains of E. coli include AIEC, DAEC, EAEC, EHEC, EIEC/Shigella, EPEC, ETEC, ExPEC, NMEC, SEPEC, ST131, or UPEC. In embodiments, the organisms include non-pathogenic bacteria. Non-limiting examples of non-pathogenic and/or beneficial bacteria include commensal bacterial, Bifidobacterium spp., Lactobacillus spp., Ruminococcus (e.g., Ruminococcus gnavus), and Akkermansia spp.

In embodiments, the organism makes up a small fraction of a complex biological sample. For example, the organism and/or the collection of organisms from which the gene sequences in each cluster are derived makes up less than about 0.5%, 1%, 2%, 3%, 4%, 5%, 6%, 7%, 8%, 9%, 10%, 15%, 20%, or 25% of the complex biological sample (e.g. of a microbial community) by relative abundance. Methods for measuring relative abundance of a microbial species within a complex biological sample (e.g., a microbiome) are known in the art. In some embodiments, polynucleotides derived from the organism and/or the collection of organisms from which the community, where the polynucleotides can be DNA, RNA, or a mix thereof, and wherein the percent can be a mass percent or a molar percent.

The methods for generating probes provided herein comprise a set cover solution. The set cover solution identifies the minimal number of probes needed to cover a cluster of gene sequences or a set of clusters of gene sequences.

Methods disclosed herein take a pan-transcriptomic or pan-genomic sequence approach to providing a probe set that identifies and facilitates the sequencing of all sequences in a large and/or variable target sequence set. For example, the methods disclosed herein are used to characterize all species of an organism, or multiple different organisms in a microbiome.

Methods disclosed herein treat each element of the “universe” in a set cover problem as being a nucleotide of a target sequence, and each element is considered “covered” as long as a probe binds to some segment of a target gene sequence that includes the element. Various methods disclosed herein first determine a hybridization pattern—i.e. where a given probe binds to a target sequence or target sequences—and then determines from those hybridization patterns the minimum number of probes needed to cover a cluster(s) of gene sequences to a degree sufficient to enable both enrichment from a sample and sequencing of any and all target gene sequences. These hybridization patterns are determined by defining certain parameters that minimize a loss function.

Probes described herein (e.g., hybrid capture probes, a candidate probe or a selected probe) comprise, for example, deoxyribonucleic acid (DNA), ribonucleic acid (RNA), peptide nucleic acid (PNA) and/or other non-naturally occurring nucleic acids.

Methods useful in the invention are described, for example, in Gnirke, et al., Nature biotechnology 27:182-189, 2009, US Patent Publication Nos. 2010/0029498, 2013/0230857, 2014/0200163, 2014/0228223, and 2015/0126377 and International Patent Publication No. WO 2009/099602).

Probes disclosed herein have about or at least about 50%, 55%, 60%, 65%, 70%, 75%, 80%, 85%, 90%, 95%, 99%, or 100% complementarity along a length thereof to gene sequences contained within the gene clusters. The length along which sequence identity is measured is at least about 10, 15, 20, 25, 30, 35, 40, 45, 50, 55, 60, 65, 70, 75, 80, 85, 80, 95, 100, 150, 200 nucleotides and/or the full length of a probe.

In embodiments, the methods of the present invention involve selecting probes to exclude from a set of probes. In embodiments, probes are excluded from the hybrid capture probe set if they contain about or at least about 25, 30, 35, 40, 45, 50, 55, 60, 65, 70, 75, 80, 85, 90, 100, 110, 120, 130, 140, or 150 contiguous nucleotides that are identical to a sequence contained in a set of reference sequences. In embodiments, probes are excluded from the hybrid capture probes if they share along a length thereof about or at least about 60%, 70%, 75%, 80%, 85%, 90%, 95%, 99%, or 100% identity to a sequence contained in a set of reference sequences. The length along which sequence identity is measured can be 10, 15, 20, 25, 30, 35, 40, 45, 50, 55, 60, 65, 70, 75, 80, 85, 80, 95, 100, 150, 200 nucleotides and/or the full length of a probe. The reference sequences can comprise genome sequences or sequences derived from a set of genome sequences. The genome sequences can be derived from organisms collectively or individually with a relative abundance of about or at least about 30%, 40%, 50%, 60%, 70%, 80%, 90%, or 95% in the microbial community. In embodiments, the genome sequences are derived from Citrobacter freundii, Salmonella enterica, or Klebsiella pneumoniae. In embodiments, the genome sequences are derived from bacteria belonging to Bacteroidetes and/or Firmicutes. Exclusion of probes can have the advantage of ensuring that sequences derived from particular organisms abundant in a microbiome are not targeted by a probe set to help increase the specificity of a probe set for targeting sequences from an organism of interest.

### Synthesis

The invention features sets of probes (e.g., hybrid capture probes) and methods for producing sets of hybrid capture probes.

In some embodiments, the invention features sets of hybrid capture probes including any one of SEQ ID NOs: 1 to 892415. In some embodiments, the invention features probes complementary to any one of SEQ ID NOs: 1 to 892415, or to any portion thereof, where non-limiting examples of portions thereof include sequences resulting from 3′ and/or 5′ truncations. The 3′ and/or 5′ truncations can be 1, 2, 3, 4, 5, 6, 7, 8, 9, or 10 nucleotide truncations. In some embodiments, the invention features probes having at least about 30%, 35%, 40%, 45%, 50%, 55%, 60%, 65%, 70%, 75%, 80%, 85%, 90%, 95%, 99%, or 100% nucleotide sequence identity to sequences provided herein (e.g., SEQ ID Nos. 1-892415). The length along which sequence identity is measured can be 10, 15, 20, 25, 30, 35, 40, 45, 50, 55, 60, 65, 70, 75, 80, 85, 80, 95, 100, 150, 200 nucleotides and/or the full length of a probe.

In embodiments, a set of probes (e.g., hybrid capture probes) contains about or at least about 10, 20, 30, 40, 50, 100, 200, 300, 400, 500, 600, 700, 800, 900, 1000, 10000, 50000, 100000, 200000, 300000, 400000, 500000, 600000, 700000, 800000, 900000, 1000000, 1500000, 2000000, 2500000, 3000000, 3500000, 4000000, 4500000, 5000000, 10,000,000 or more unique probes. In embodiments, the set of hybrid capture probes contains no more than about 100000, 200000, 300000, 400000, 500000, 600000, 700000, 800000, 900000, 1000000, 1500000, 2000000, 2500000, 3000000, 3500000, 4000000, 4500000, 5000000, 10,000,000 or more unique probes. In embodiments, each unique probe in the set of probes contains a distinct probe sequence.

Methods for synthesizing a set of hybrid capture probes involve synthesis of oligonucleotides in an array format (e.g., chip). Array synthesis can have the advantages of being customizable and capable of producing long oligonucleotides. TWIST chemistry can also be used to manufacture a set of hybrid capture probes.

In certain embodiments, the probes contain a binding member. In certain example embodiments, the binding member is biotin, a hapten, or an affinity tag. In cases where the hybrid capture probes are biotinylated, the capture probes are captured using a capture molecule (e.g., streptavidin) fixed to a solid support. The capture molecule and/or the binding member can be streptavidin, biotin, a hapten, an affinity tag, an antigen-binding molecule, or an antigen. The hybrid capture probes and/or the solid support can contain more than one distinct binding member or capture molecule, respectively.

The solid support can comprise metal, glass, a polymeric material, or any other suitable material. The support can be planar and/or the support can contain particles or beads. The support can be a biochip. The capture molecule can be coupled to the support by covalent or non-covalent bonds. In some embodiments, hybrid capture probes are directly or indirectly covalently coupled to the solid support.

In other embodiments, the set of probes (e.g., hybrid capture probes) are produced using methods described herein or known to the skilled person. In embodiments, the probes of the present invention include mixed or universal nucleotides, such as inosine or 5-nitroindole (i.e., degeneracy). The mixed or universal base(s) can be included in the bait sequence at the position(s) of a single nucleotide polymorphism (SNP) or mutation, to optimize the bait sequences to catch both alleles (i.e., SNP and non-SNP; mutant and non-mutant). In other embodiments, all known sequence variations (or a subset thereof) can be targeted with multiple probes, rather than by using mixed degenerate probes.

In embodiments, the set of hybrid capture probes are derived from oligonucleotides synthesized in a microarray and cleaved and eluted from the microarray.

In some embodiments, the hybrid capture probes are RNA and/or DNA molecules, as well as derivatives or analogs thereof. In some embodiments the probes are chemically or enzymatically modified or in vitro transcribed RNA molecules including but not limited to those that are more stable and resistant to RNase.

In embodiments, the probes (e.g., hybrid capture probes) comprise about 10, 15, 20, 25, 30, 35, 40, 45, 50, 55, 60, 65, 70, 75, 80, 85, 90, 95, 100, 110, 120, 130, 140, 150, 160, 170, 180, 190, or 200 nucleotides. It can be beneficial in some contexts to use hybrid capture probes having a nucleotide length of no more than about 80, 85, 90, 95, 100, 110, 120, 130, 140, 150, 160, 170, 180, 190, 200, or 250 nucleotides.

In some embodiments, probes generated according to the methods described herein contain non-naturally occurring linkages such as locked nucleic acid (“LNA”) or peptide nucleotide acids.

### Hybrid Capture (HC)

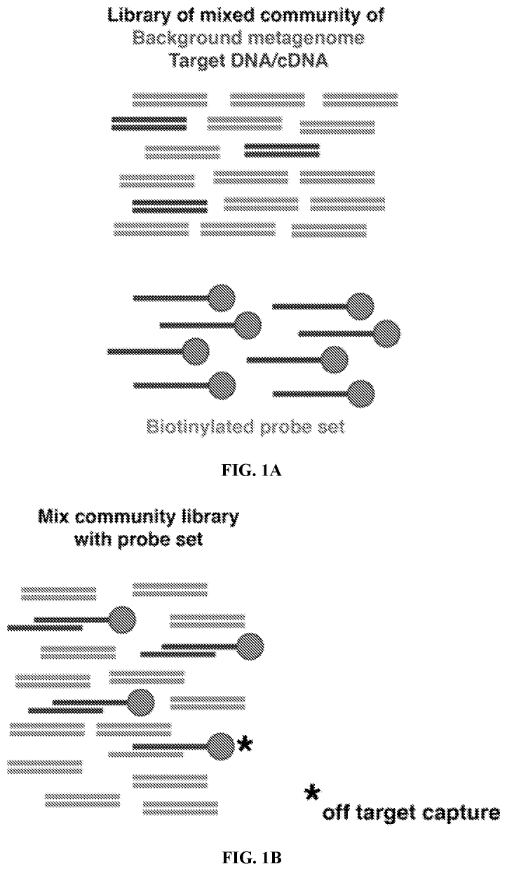

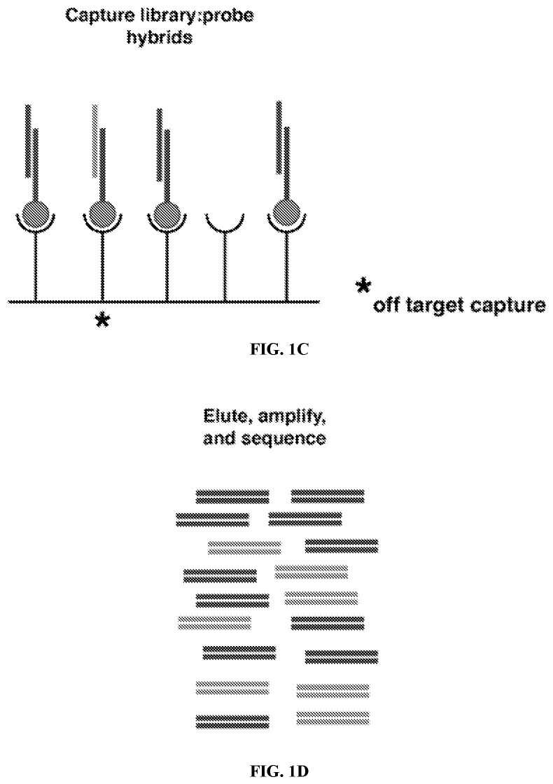

Hybrid capture (HC), also called hybrid selection, relies on specific oligonucleotides (i.e., capture probes or simply “probes”) that selectively hybridize (i.e., bind or capture) to sequences from the target organism (FIGS. 1A-1D) (Mamanova et al., 2010. “Target-Enrichment Strategies for next-Generation Sequencing.” Nature Methods 7 (2): 111-18, which is incorporated by reference in its entirety).

Hybridization between the polynucleotides and hybrid capture probes is conducted under any conditions in which the hybrid capture probes hybridize to target polynucleotides, but do not substantially hybridize to non-target polynucleotides. This can involve selection under high stringency conditions. Following hybridization, the polynucleotide/probe complexes are separated based on the presence of a binding member in each probe, and unbound polynucleotides are removed under appropriate wash conditions that remove the nonspecifically bound polynucleotides, but do not substantially remove polynucleotide probe complexes.

In one embodiment, hybrid capture is carried out using methods including those described herein and those described in Gnirke, et al., Nature biotechnology 27:182-189, 2009, US patent publications No. US 2010/0029498, US 2013/0230857, US 2014/0200163, US 2014/0228223, and US 2015/0126377 and International Patent Publication No. WO 2009/099602, each of which is incorporated by reference in its entirety.

For example, the invention encompasses use of hybrid capture probes of the present invention with the SureSelectXT, SureSelectXT2 and SureSelectQXT Target Enrichment System, the SeqCap EZ kit developed by Roche NimbleGen, a TruSeq® Enrichment Kit developed by Illumina, and other hybridization-based target enrichment methods and kits that add sample-specific sequence tags either before or after the enrichment step.

The hybrid capture methods provided herein can be used for enriching pan-genomic or pan-transcriptomic polynucleotides. The polynucleotides can be related by structure or by function. The polynucleotides can be associated with one or more functions of interest. The pan-genomic or pan-transcriptomic polynucleotides can be enriched by about or at least about 1, 1.1, 1.2, 1.3, 1.4, 1.5, 2, 3, 4, 5, 6, 7, 8, 9, 10, 15, 20, 25, 30, 35, 40, 45, 50, 55, 60, 65, 70, 75, 80, 85, 90, 95, or 100-fold.

In embodiments, the hybrid capture methods provided herein can be used for enriching for polynucleotides derived from an organism(s) of interest from a host organism (e.g., an animal or human). Thus, the probes of the present invention can be used in a method to separate sequences derived from an organism(s) of interest (e.g., a bacterium) from a host organism(s) (e.g., a human). Therefore, enrichment of the sequences derived from the organism(s) of interest can involve increasing the concentration of polynucleotides derived from the organism(s) of interest in a sample relative to the concentration of polynucleotides derived from host organism(s) in the sample by about or at least about 1, 1.1, 1.2, 1.3, 1.4, 1.5, 2, 3, 4, 5, 6, 7, 8, 9, 10, 15, 20, 25, 30, 35, 40, 45, 50, 55, 60, 65, 70, 75, 80, 85, 90, 95, or 100-fold. As a non-limiting example, the probes and methods of the present disclosure can be used to separate polynucleotides derived from a tumorigenic bacterium from tumor-derived polynucleotides in a tumor sample.

The hybrid capture probes in various embodiments amplify targeted sequences in a non-biased manner. In some cases, the capture probes minimally amplify non-target sequences. As detailed further in the below Examples, the methods of the present invention utilizing hybrid capture probes of the present invention can be used to enrich for target sequences in a polynucleotide sample while still maintaining a non-biased representation in the sample of relative abundances of non-targeted nucleotide sequences. Thus, in embodiments, the methods of the present invention result in enriched polynucleotide compositions where target sequences are enriched in a substantially non-biased manner and where non-target sequences are reduced relative to the enriched target sequences in a substantially non-biased manner. In embodiments, a non-substantial bias means a lack of any measured statistically significant bias.

In embodiments, conditions (e.g., salt concentration and/or temperature) are adjusted such that hybridization between a target sequence and a hybridization probe(s), optionally bound to a solid support, occurs with precise complementary matches or with various degrees of less complementarity depending on the degree of stringency employed. For example, stringent salt concentration can include those containing less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be achieved in the absence of organic solvent, e.g., formamide, while high stringency hybridization can be obtained in the presence of at least about 35% formamide, and most preferably at least about 50% formamide. Stringent temperature conditions can include temperatures of at least about 30° C., of at least about 37° C., or of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed.

### Samples

The methods of the present invention can be used to characterize virtually any complex biological sample (i.e., samples containing polynucleotides derived from more than one organism). In particular, the probes and methods described herein can be used to enrich polynucleotides (e.g., those polynucleotides related by sequence or function) derived from any sample containing a microbe or a microbial community (e.g., a microbiome). The microbial community is a natural or artificial/synthetic microbial community. The microbial community can be grown ex vivo situ or in vivo situ. The sample can be an environmental or biological sample. The sample can be obtained from a subject. The sample can be obtained from a subject at different time points. The samples can be collected from a subject suffering or having suffered an infection (e.g., a urinary tract infection).

The samples can be gathered from a patient care facility (e.g., a hospital) or a food production facility and characterization of the sample can include monitoring an organism(s) (e.g., a microbe) and/or microbial community within the samples. Characterization of the sample can include monitoring or detecting the presence and/or levels of polynucleotides derived from the sample and associated with a function of interest. The sample can be collected from an organoid. In various instances, the sample can comprise a limited number of microbial species and/or genera; for example, 1, 2, 3, 4, 5, 6, 7, 8, 9, 10, 50, 100, or 500 species and/or genera.

Some prokaryotic species are known to be tumorigenic. It can be advantageous, therefore, to characterize microbes associated with a tumor sample. Thus, in some embodiments, the sample can contain a biopsy (e.g., a tumor biopsy). In instances, prokaryotes cause a cancer in the gut of a subject.

Methods for preparing libraries of polynucleotides for sequencing are known to one of skill in the art. Library preparation can include the addition of nucleotide bar codes to the library polynucleotides according to methods known in the art. Libraries can be prepared using commercially available kits. The libraries can contain polynucleotides having a length or an average length of about or of at least about 25, 30, 35, 40, 45, 50, 55, 60, 65, 70, 75, 80, 85, 90, 95, 100, 125, 150, 175, 200, 250, 300, 350, 400, 450, 500, or 1000 nucleotides. In embodiments, the libraries contain polynucleotides having an a length or average length of no more than about 25, 30, 35, 40, 45, 50, 55, 60, 65, 70, 75, 80, 85, 90, 95, 100, 125, 150, 175, 200, 250, 300, 350, 400, 450, 500, or 1000 nucleotides. Not wishing to be bound by theory, libraries of larger length can have the advantage of allowing for hybrid capture of sequences outside of those binding to the hybrid capture probes by means of a neighborhood effect.

### Characterization

The hybrid capture probes and methods featured in the disclosure can be used for the characterization of a microbe and/or microbiome. The hybrid capture probes can be used to characterize a complex biological sample and/or microbiome which may comprise a target sequence(s) or a fragment thereof. The target sequences can be related by structure and/or function. The method may comprise (a) contacting the selected probes to the target sequence or a fragment thereof; and (b) analyzing the target sequence or fragment thereof that hybridizes to one or more of the selected probes.

Analyzing the target sequence or fragment thereof that hybridizes to one or more of the selected probes may involve sequencing, FACS, qPCR, RT-PCR, a genotyping array, and/or a NanoString assay (see, e.g., Malkov, et al. “Multiplexed measurements of gene signatures in different analytes using the Nanostring nCounter™ Assay System”, BMC Research Notes, 2: Article No: 80 (2009)), or any of various other techniques known to one of skill in the art. Various characterization methods may be used and are described as follows.

RNA sequencing (RNA-Seq) is a powerful tool for transcriptome profiling. In embodiments, to mitigate sequence-dependent bias resulting from amplification complications to allow truly digital RNA-Seq, a set of barcode sequences can be used to ensure that every cDNA molecule prepared from an mRNA sample is uniquely labeled by random attachment of barcode sequences to both ends (see, e.g., Shiroguchi K, et al. Proc Natl Acad Sci USA. 2012 Jan. 24;109(4):1347-52). After PCR, paired-end deep sequencing can be applied to read the two barcodes and cDNA sequences. Rather than counting the number of reads, RNA abundance can be measured based on the number of unique barcode sequences observed for a given cDNA sequence. The barcodes may be optimized to be unambiguously identifiable. This method is a representative example of how to quantify a whole transcriptome from a sample.

Library preparation may involve an amplification step. Amplification may involve thermocycling or isothermal amplification (such as through the methods RPA or LAMP). Cross-linking may involve overlap-extension PCR or use of ligase to associate multiple amplification products with each other. Amplification can refer to any method employing a primer and a polymerase capable of replicating a target sequence with reasonable fidelity. Amplification may be carried out by natural or recombinant DNA polymerases such as TaqGold™, T7 DNA polymerase, Klenow fragment of E. coli DNA polymerase, and reverse transcriptase. A preferred amplification method is PCR. In particular, the isolated RNA can be subjected to a reverse transcription assay that is coupled with a quantitative polymerase chain reaction (RT-PCR) in order to quantify the expression level of a sequence associated with a signaling biochemical pathway.

Detection of the gene expression level can be conducted in real time in an amplification assay. In one aspect, the amplified products can be directly visualized with fluorescent DNA-binding agents including but not limited to DNA intercalators and DNA groove binders. Because the amount of the intercalators incorporated into the double-stranded DNA molecules is typically proportional to the amount of the amplified DNA products, one can conveniently determine the amount of the amplified products by quantifying the fluorescence of the intercalated dye using conventional optical systems in the art. DNA-binding dyes suitable for this application include, as non-limiting examples, SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, and the like.

In another aspect, other fluorescent labels such as sequence specific probes can be employed in the amplification reaction to facilitate the detection and quantification of the amplified products. Probe-based quantitative amplification relies on the sequence-specific detection of a desired amplified product. It utilizes fluorescent, target-specific probes (e.g., TaqMan® probes) resulting in increased specificity and sensitivity. Methods for performing probe-based quantitative amplification are taught, for example, in U.S. Pat. No. 5,210,015.

Sequencing may be performed on any high-throughput platform. Methods of sequencing oligonucleotides and nucleic acids are well known in the art (see, e.g., WO93/23564,WO98/28440 and WO98/13523; U.S. Pat. App. Pub. No. 2019/0078232; U.S. Pat. Nos. 5,525,464; 5,202,231; 5,695,940; 4,971,903; 5,902,723; 5,795,782; 5,547,839 and 5,403,708; Sanger et al., Proc. Natl. Acad. Sci. USA 74:5463 (1977); Drmanac et al., Genomics 4:114 (1989); Koster et al., Nature Biotechnology 14:1123 (1996); Hyman, Anal. Biochem. 174:423 (1988); Rosenthal, International Patent Application Publication 761107 (1989); Metzker et al., Nucl. Acids Res. 22:4259 (1994); Jones, Biotechniques 22:938 (1997); Ronaghi et al., Anal. Biochem. 242:84 (1996); Ronaghi et al., Science 281:363 (1998); Nyren et al., Anal. Biochem. 151:504 (1985); Canard and Arzumanov, Gene 11:1 (1994); Dyatkina and Arzumanov, Nucleic Acids Symp Ser 18:117 (1987); Johnson et al., Anal. Biochem. 136:192 (1984); and Elgen and Rigler, Proc. Natl. Acad. Sci. USA 91 (13): 5740 (1994), all of which are expressly incorporated by reference).

The sequencing of a polynucleotide can be carried out using any suitable commercially available sequencing technology. In another embodiment, the sequencing of a polynucleotide is carried out using chain termination method of DNA sequencing (e.g., Sanger sequencing). In yet another embodiment, commercially available sequencing technology is a next-generation sequencing technology, including as non-limiting examples combinatorial probe anchor synthesis (cPAS), DNA nanoball sequencing, droplet-based or digital microfluidics, heliscope single molecule sequencing, nanopore sequencing (e.g., Oxford Nanopore technologies), GeneGap sequencing, massively parallel signature sequencing (MPSS), microfluidic Sanger sequencing, microscopy-based techniques (e.g., transmission electronic microscopy DNA sequencing), RNA polymerase (RNAP) sequencing, single-molecule real-time (SMRT) sequencing, SOLID sequencing, ion semiconductor sequencing, polony sequencing, Pyrosequencing (454), sequencing by hybridization, sequencing by synthesis (e.g., Illumina™ sequencing), sequencing with mass spectrometry, and tunneling currents DNA sequencing.

Polynucleotides may be characterized and/or enriched by means of a biochip (also known as a microarray) containing hybrid capture probes of the present invention. Biochips generally comprise solid substrates and have a generally planar surface, to which a capture reagent (also called an adsorbent or affinity reagent) is attached. The capture reagent can be a hybrid capture probe(s) or a binding member. Frequently, the surface of a biochip comprises a plurality of addressable locations, each of which has the capture reagent bound there.

The array elements are organized in an ordered fashion such that each element is present at a specified location on the substrate. Useful substrate materials include membranes, composed of paper, nylon or other materials, filters, chips, glass slides, and other solid supports. Such solid supports are suitable for use as solid supports generally in embodiments of the present invention. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996), herein incorporated by reference. Methods for making polypeptide microarrays are described, for example, by Ge (Nucleic Acids Res. 28: e3. i-e3. vii, 2000), MacBeath et al., (Science 289:1760-1763, 2000), Zhu et al. (Nature Genet. 26:283-289), and in U.S. Pat. No. 6,436,665, hereby incorporated by reference.

In aspects of the invention, a sample is analyzed by means of a nucleic acid biochip (also known as a nucleic acid microarray). To produce a nucleic acid biochip, oligonucleotides may be synthesized or bound to the surface of a substrate using a chemical coupling procedure and an ink jet application apparatus, as described in PCT application W095/251116 (Baldeschweiler et al.). Alternatively, a gridded array may be used to arrange and link cDNA fragments or oligonucleotides to the surface of a substrate using a vacuum system, thermal, UV, mechanical or chemical bonding procedure.

Detection system for measuring the absence, presence, and amount of hybridization for all of the distinct nucleic acid sequences are well known in the art. For example, simultaneous detection is described in Heller et al., Proc. Natl. Acad. Sci. 94:2150-2155, 1997. In embodiments, a scanner is used to determine the levels and patterns of fluorescence.

### Molecular Identifiers

For a convenient detection of polynucleotide/probe complexes, the hybrid capture probes can be coupled to a molecular identifier. Molecular identifiers suitable for use in the present invention include any agent detectable by photochemical, biochemical, spectroscopic, immunochemical, electrical, optical or chemical means. In some embodiments, a probe described herein is linked to a nucleotide sequence that is used for molecular identification.

A wide variety of appropriate molecular identifiers are known in the art, which include fluorescent or chemiluminescent labels, radioactive isotope labels, enzymatic or other ligands. The molecular identifier can be a fluorescent label or an enzyme tag, such as digoxigenin, β-galactosidase, urease, alkaline phosphatase or peroxidase, avidin/biotin complex.

Methods used to detect or quantify the hybridization intensity will typically depend upon the molecular identifier. For example, radiolabels may be detected using photographic film or a phosphoimager. Fluorescent markers may be detected and quantified using a photodetector to detect emitted light. Enzymatic labels can be detected by providing the enzyme with a substrate and measuring the reaction product produced by the action of the enzyme on the substrate; and colorimetric labels can be detected by visualizing a colored label.

Specific non-limiting examples of molecular identifiers include radioisotopes, such as 32P, 14C, 125I, 3H, and 131I, fluorescein, rhodamine, dansyl chloride, umbelliferone, luciferase, peroxidase, alkaline phosphatase, β-galactosidase, β-glucosidase, horseradish peroxidase, glucoamylase, lysozyme, saccharide oxidase, microperoxidase, biotin, and ruthenium. In the case where biotin is employed as a molecular identifier, streptavidin bound to an enzyme (e.g., peroxidase) may further be added to facilitate detection of the biotin.

Examples of fluorescent molecular identifiers include, but are not limited to, Atto dyes, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinyl sulfonyl)phenyl]naphthalimide-3,5 disulfonate; N-(4-anilino-1-naphthyl) maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino] naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′ tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine

A fluorescent molecular identifier may be a fluorescent protein, such as blue fluorescent protein, cyan fluorescent protein, green fluorescent protein, red fluorescent protein, yellow fluorescent protein or any photoconvertible protein. Colorimetric molecular identifiers, bioluminescent molecular identifiers and/or chemiluminescent molecular identifiers may be used in embodiments of the invention.

Detection of a molecular identifier may involve detecting energy transfer between molecules in a hybridization complex by perturbation analysis, quenching, or electron transport between donor and acceptor molecules, the latter of which may be facilitated by double stranded match hybridization complexes. The fluorescent molecular identifier may be a perylene or a terrylen. In the alternative, the fluorescent molecular identifier may be a fluorescent bar code.

The molecular identifier may be light sensitive, wherein the label is light-activated and/or light cleaves the one or more linkers to release the molecular cargo. The light-activated molecular cargo may be a major light-harvesting complex (LHCII). In another embodiment, the fluorescent molecular label may induce free radical formation.

In an advantageous embodiment, agents may be uniquely labeled in a dynamic manner (see, e.g., international patent application serial no. PCT/US2013/61182 filed Sep. 23, 2012). The unique labels are, at least in part, nucleic acid in nature, and may be generated by sequentially attaching two or more detectable oligonucleotide tags to each other and each unique label may be associated with a separate agent. A detectable oligonucleotide tag may be an oligonucleotide that may be detected by sequencing of its nucleotide sequence and/or by detecting non-nucleic acid detectable moieties to which it may be attached.

In embodiments, the molecular identifier is a microparticles including as non-limiting examples quantum dots (Empodocles, et al., Nature 399:126-130, 1999), gold nanoparticles (Reichert et al., Anal. Chem. 72:6025-6029, 2000).

Characterizing a target sequence or fragment thereof that hybridizes to one or more of the hybrid capture probes may be an identifying analysis, wherein hybridization of a selected hybrid capture probe(s) to the target sequence or a fragment thereof indicates the presence of the target sequence within the sample.

### Hardware and Software

The present invention also relates to a computer system involved in carrying out the methods of the invention relating to both computations and sequencing.

A computer system (or digital device) may be used to receive, transmit, display and/or store results, analyze the results, and/or produce a report of the results and analysis. A computer system may be understood as a logical apparatus that can read instructions from media (e.g. software) and/or network port (e.g. from the internet), which can optionally be connected to a server having fixed media. A computer system may comprise one or more of a CPU, disk drives, input devices such as keyboard and/or mouse, and a display (e.g. a monitor). Data communication, such as transmission of instructions or reports, can be achieved through a communication medium to a server at a local or a remote location. The communication medium can include any means of transmitting and/or receiving data. For example, the communication medium can be a network connection, a wireless connection, or an internet connection. Such a connection can provide for communication over the World Wide Web. It is envisioned that data relating to the present invention can be transmitted over such networks or connections (or any other suitable means for transmitting information, including but not limited to mailing a physical report, such as a print-out) for reception and/or for review by a receiver. One can record results of calculations (e.g., sequence analysis or a listing of hybrid capture probe sequences) made by a computer on tangible medium, for example, in computer-readable format such as a memory drive or disk, as an output displayed on a computer monitor or other monitor, or simply printed on paper. The results can be reported on a computer screen. The receiver can be but is not limited to an individual, or electronic system (e.g. one or more computers, and/or one or more servers).

In some embodiments, the computer system may comprise one or more processors. Processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, or other suitable storage medium. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

A client-server, relational database architecture can be used in embodiments of the invention. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments of the invention, the server computer handles all of the database functionality. The client computer can have software that handles all the front-end data management and can also receive data input from users.

A machine readable medium which may comprise computer-executable code may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The subject computer-executable code can be executed on any suitable device which may comprise a processor, including a server, a PC, or a mobile device such as a smartphone or tablet. Any controller or computer optionally includes a monitor, which can be a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard, mouse, or touch-sensitive screen, optionally provide for input from a user. The computer can include appropriate software for receiving user instructions, either in the form of user input into a set of parameter fields, e.g., in a GUI, or in the form of preprogrammed instructions, e.g., preprogrammed for a variety of different specific operations.

### Kits

The instant disclosure also provides kits containing agents of this disclosure for use in the methods of the present disclosure. Kits of the instant disclosure may include one or more containers comprising an agent for characterization of a complex biological sample (e.g. a microbiome) and/or may contain agents (e.g., oligonucleotide primers, probes, etc.) for enrichment of sequences derived from a complex biological sample. In some embodiments, the kits further include instructions for use in accordance with the methods of this disclosure. In some embodiments, these instructions comprise a description of use of the agent to characterize a complex biological sample and/or enrich polynucleotide sequences according to any of the methods of this disclosure. In some embodiments, the instructions comprise a description of how to enrich polynucleotides from a sample and/or to characterize a complex biological sample (e.g., a microbiome). The kit may further comprise a description of how to analyze and/or interpret data.

Instructions supplied in the kits of the instant disclosure are typically written instructions on a label or package insert (e.g., a paper sheet included in the kit), but machine-readable instructions (e.g., instructions carried on a magnetic or optical storage disk) are also acceptable. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. Kits may optionally provide additional components such as buffers and interpretive information. Normally, the kit comprises a container and a label or package insert(s) on or associated with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Design and initial validation of E. coli pan-genome probe set

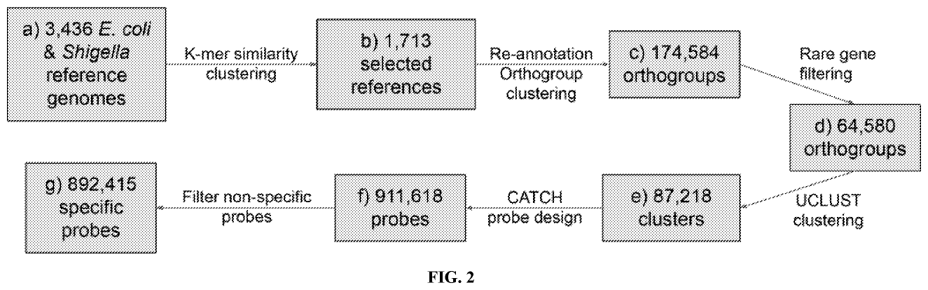

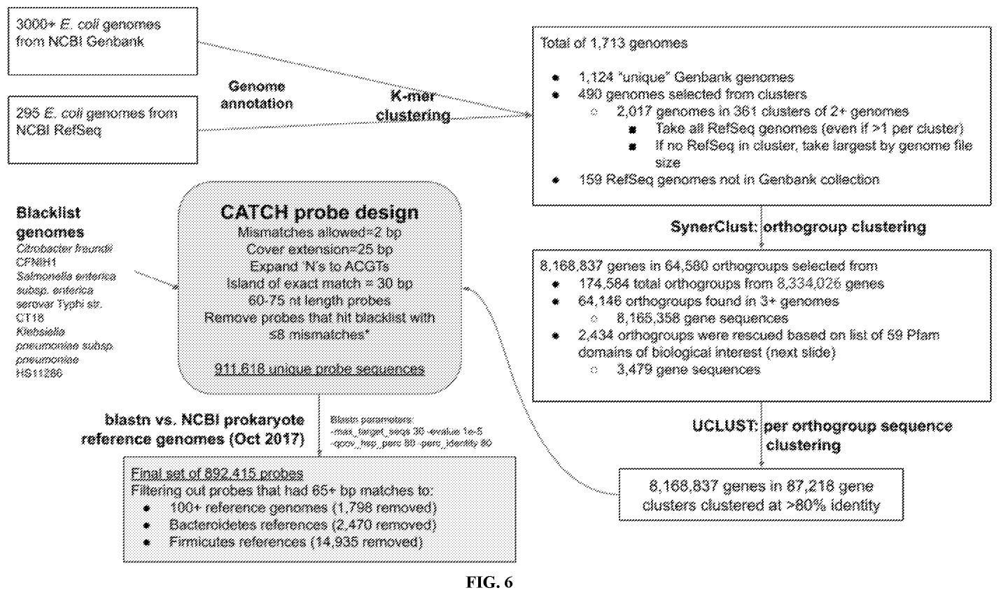

In order to design a probe set to cover the full scope of genetic diversity present in E. coli, an evolutionarily and functionally diverse species composed of eight main phylogroups (A, B1, B2, C, D, E, F, and G), and which also contains Shigella nested within its phylogeny, 295 RefSeq complete E. coli reference genomes and 3,141 high quality E. coli reference genomes were obtained from Genbank that were listed in the NCBI Pathogen Detection database. After applying a k-mer based clustering algorithm to remove near-identical reference genomes, shared orthogroups were identified. To reduce computational runtime on the probe design, orthogroup genes were grouped into clusters with >80% nucleotide sequence identity, and then these clusters were processed using the CATCH algorithm (Metsky, Hayden C., Viral Hemorrhagic Fever Consortium, Katherine J. Siddle, Adrianne Gladden-Young, James Qu, David K. Yang, Patrick Brehio, et al. 2019. “Capturing Sequence Diversity in Metagenomes with Comprehensive and Scalable Probe Design.” Nature Biotechnology. doi.org/10.1038/s41587-018-0006-x), followed by filtering steps to remove probes likely to target non-E. coli sequences. The remaining set of 892,415 short oligonucleotide probes (provided in the Sequence Listing as SEQ ID NOs: 1 to 892415) was designed to specifically hybridize to and cover the E. coli pan-genome, including the vast majority of the known diversity of E. coli (FIGS. 2 and 6).

In order to confirm the coverage of the final probe set against the E. coli pan-genome, blastn was used to query the probe sequences against all protein-coding gene sequences of a set of 1,713 references (see Table 2), including genes that had previously been filtered out at the probe design stage. The probe set was considered as capturing a gene sequence if at least one probe had a blast hit of at least >65 bp and no more than 8 mis-matches to a given gene. The final probe set successfully captured 99.95% of the genes included in the design (8,131,231 genes) and 99.54% of the included orthogroups (52,688 orthogroups). Thus, the probe set targeted the vast majority of the diversity of E. coli genes and orthogroups, even after removing thousands of potentially off-target probes. Of the missing genes and orthogroups, 67% were annotated hypothetical proteins, while the remaining genes were annotated as associated with mobile genetic elements. In addition, the probe set captured 29,232 E. coli genes that had been filtered out prior to probe design, suggesting that the probe set was more generalizable and actually able to capture additional sequence diversity outside the genes and orthogroups included in the design process. Probes were designed to selectively and sensitively enrich for genes without strain or reference bias. 892,415 oligonucleotide probes were generated (provided in the Sequence Listing as SEQ ID NOs: 1 to 892415).

TABLE 2
Phylogroup distribution of the 1,713 reference
genomes used in probe design.

| Phylogroup | References |
|---|---|
| A | 410 |
| B1 | 548 |
| B2 | 386 |
| C | 48 |
| D | 146 |
| E | 93 |
| F | 31 |
| G | 16 |
| Shigella | 20 |
| Cryptic clades* | 11 |
| E. albertii† | 1 |
| Unknown‡ | 3 |

*Unnamed lineages in the Escherichia genus (Walk, Seth T., Elizabeth W. Alm, David M. Gordon, Jeffrey L. Ram, Gary A. Toranzos, James M. Tiedje, and Thomas S. Whittam. 2009. “Cryptic Lineages of the Genus Escherichia.” Applied and Environmental Microbiology 75 (20): 6534-44)
†Reference GCA_001286085.1 was mis-labeled as E. coli in NCBI
‡Could not determine phylogroup using ClermonTyping

### Example 2: Enrichment of 4 Strains of E. coli in a Mock Metagenomic Community

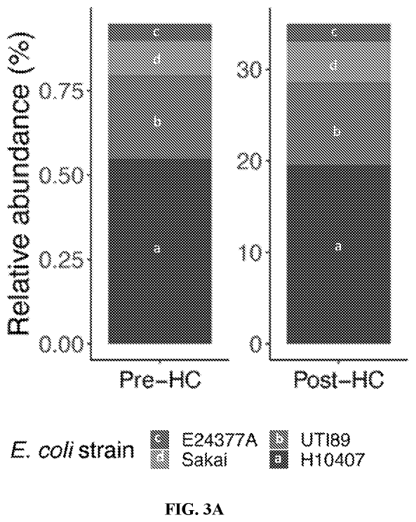

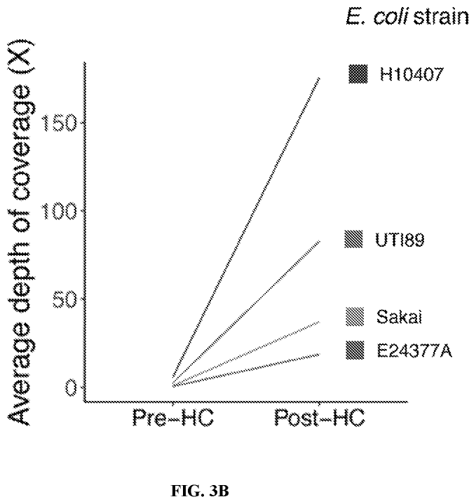

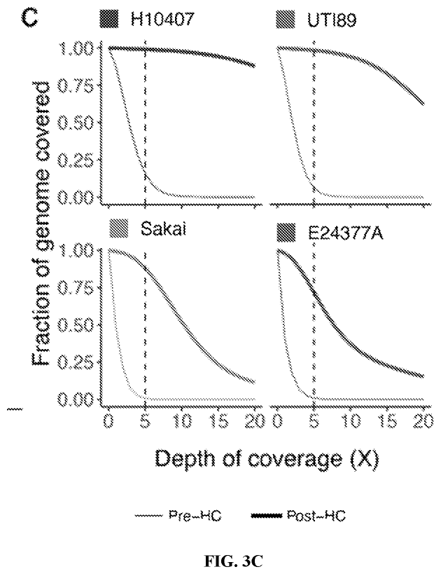

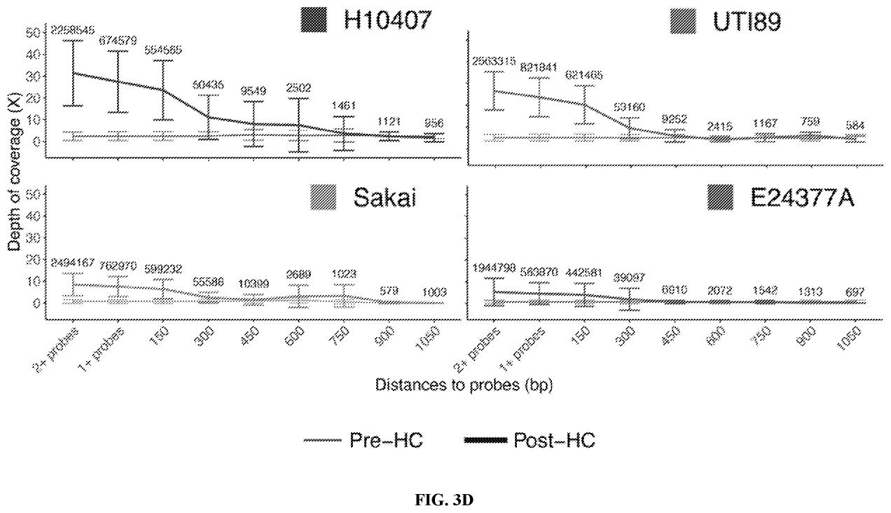

In order to assess the performance of the probe set on a real sample of known composition, a mock community was created and sequenced containing approximately 99% human DNA and 1% E. coli DNA, representing a mixture of four distinct, previously sequenced E. coli strains from different phylogroups, with fully finished reference genomes mixed in unequal (80:15:4.9:0.1) relative abundances (Dijk, Lucas R. van, Bruce J. Walker, Timothy J. Straub, Colin J. Worby, Alexandra Grote, Henry L. Schreiber, Christine Anyansi, et al. 2021. “StrainGE: A Toolkit to Track and Characterize Low-Abundance Strains in Complex Microbial Communities.” Cold Spring Harbor Laboratory. doi.org/10.1101/2021.02.14.431013). A sequencing library was created for this sample and the hybrid capture (HC) protocol was performed using the pan-genome probe set. Both pre-HC and post-HC libraries were sequenced and analyzed for enrichment and bias for each of the four strains (FIGS. 3A-3D). StrainGE, a tool developed for disentangling low-abundance mixtures of strains from within the same species in metagenomic samples, was used to assess performance for each of the four strains individually (Dijk et al. 2021).

After HC, relative ratios of the four strains to each other were well maintained in the sequencing data (paired t-test, p>0.1, FIG. 3A)-while their relative abundances within the overall metagenomic sample were dramatically increased (one sample t-test of log 2 fold-changes for all four strains, p<0.001), with nearly a 40-fold increase overall. Therefore, enrichment did not introduce bias, as shown by the preservation of relative strain ratios in the post-hybrid capture data (paired t-test, >0.1). The depth of coverage for each of the four strains was also dramatically increased for the same sequencing depth. The most abundant strain increased from 6× coverage to nearly 200× coverage (FIG. 3B), and the least abundant strain (originally only present at 0.05% relative abundance) increased from 0.6× to 27× coverage. Due to increased coverage for each strain, the fraction of the genome covered was also dramatically increased (FIG. 3C). In post-HC data (thick lines), the majority of each strain's reference was covered at least 5× (a standard minimum cutoff for depth; dashed vertical line)—ranging from 99% for the most abundant strain to 72% for the least abundant strain. This represented a dramatic increase compared to pre-HC data (thin lines), in which little of the two more abundant strains (16% and 6%) and nearly none (1% and 0.8%) of the two less abundant strains had at least 5× coverage.

Although the probe set was designed to cover the E. coli pan-genome as evenly as possible, it was expected that there would be some regions of lower coverage due to their not being included in the probe set design. Due to computational and practical purposes, certain genes were excluded from consideration in the original probe design, including genes and orthogroups found rarely in E. coli, as well as sequences found commonly in other gut bacteria. Further, though E. coli genomes are generally quite gene-dense (>95% genic), intergenic regions were not considered in the probe design process; thus, it was expected that intergenic regions might have lower coverage.

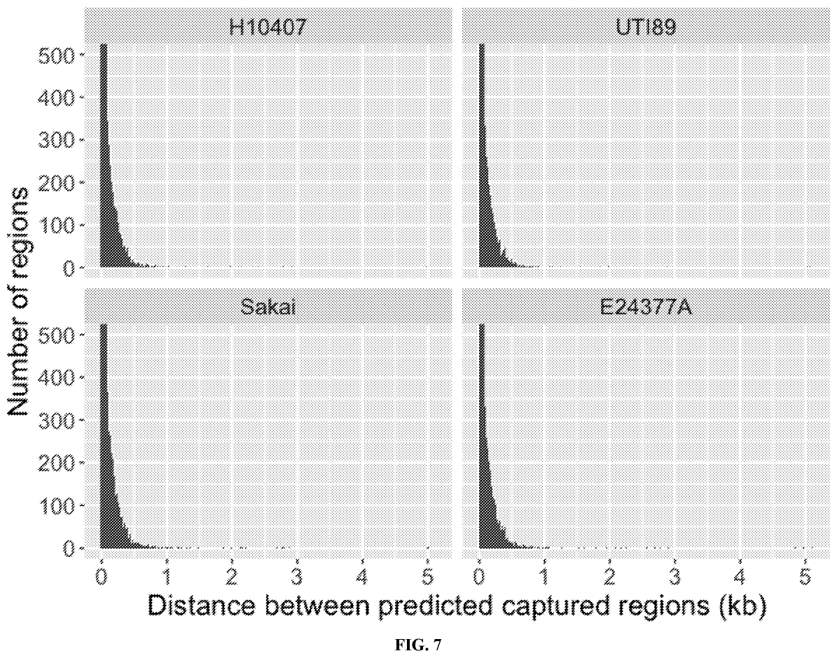

In order to assess the extent of regions expected to be missed by the probe set in the four reference genomes in the mock community, probe hybridization sites were predicted for each strain and uncaptured regions (i.e., gaps) were characterized (FIG. 7). Most of each reference was relatively well-covered by predicted probe hybridization sites, with between 83% and 86% of each genome predicted to hybridize directly to the probe set. There were 30,076 small (<50 bp) gaps between sites, accounting for a total of 545 kb of sequence (˜3%), across all four strains. This was not unexpected, as CATCH was used with a cover extension value of 25 bp, which caused the algorithm to assume that a probe will capture sequences within 25 bp (on each end) of a given hybridization site. There were 17,042 medium-sized gaps (between 50 bp and 1 kb) across all four references, amounting to a total of 2.37 Mb of sequence (˜12%), which were not necessarily expected to be captured by the probes. There were 81 large gaps (between 1 kb and 5.1 kb), which amounted to a total of 181 kb of sequence (0.9%). Thus, somewhere between 1% and 13% of sequence from these four genomes was expected to go uncaptured, and therefore, unenriched. Notably, the distribution of gap sizes was not significantly different between the four strains (Kruskal Wallis, p>0.1), suggesting limited inter-strain bias in the potentially uncaptured gene content.

These predictions were then compared to actual post-HC sequence data, to see how much sequence was actually captured or missed by the probes. The probes appeared to enrich sequences well beyond 25 bp from hybridization sites (FIG. 3D). Increases were observed in coverage for post-HC data (thick lines) over pre-HC data (thin lines) for regions of the reference up to approximately 300 bp away from predicted probe hybridization sites, which was similar to the average library insert size. Coverage levels for post-HC decreased to pre-HC levels around 450 bp from predicted probe hybridization sites, indicating that these positions in the genome were covered only at background levels, which was expected for non-enriched sequences. In total, between 0.4% and 3.1% of sequence from these four genomes was unenriched following capture, which was a lower range than expected.

Finally, as there was a high depth of coverage, an attempt was made to assemble the four strains of E. coli using a metagenomic assembler, MetaSPAdes (Nurk, Sergey, Dmitry Meleshko, Anton Korobeynikov, and Pavel A. Pevzner. 2017. “metaSPAdes: A New Versatile Metagenomic Assembler.” Genome Research 27 (5): 824-34). Basic assembly metrics for pre-HC and post-HC data are shown in Table 1. Substantially more E. coli sequence was assembled from the post-HC sequence data, as compared to the pre-HC sequence data. The pre-HC assembly was only 3.9 Mb in total length (less than the length of a single E. coli genome), and was very fragmented, with 2,107 contigs >1 kb in length. The assembly (7.63 Mb) was longer in length than that of the pre-HC assembly, and it was more contiguous (1,602 contigs >1 kb, including one contig that was nearly 60 kb in length).

TABLE 1
Post-HC E. coli assembly is more contiguous and larger.
Data from contigs that were assigned to E. coli based
on taxonomy of blastn results to nt are reported.

| | | | | Number of |
|---|---|---|---|---|
| | Contig N50 | Largest contig | Assembly size* | Contigs** |
| Pre-HC | 2,048 bp | 21.6 kb | 3.90 Mb | 2,107 |
| Post-HC | 7,073 bp | 58.8 kb | 7.63 Mb | 1,602 |

*It is likely that more than one genome was assembled to constitute the Post-HC assembly, as there were four E. coli strains, each with a genome approximately only 5 Mb in size.
**Only including contigs >1 kb in length

### Example 3: Enrichment of E. coli DNA From Metagenomic Libraries Derived From Human Stool Samples

Having characterized the behavior of the probe set on a known mixture of E. coli, the technology was next applied to stool samples from healthy human volunteers. Stool was collected monthly from a number of participants over the course of a year (10 samples were selected for use in this study, including some samples from multiple time points from the same participant). Multiple E. coli strains were present in the stool of many of the volunteers. Both DNA and RNA were extracted, libraries were created, and E. coli content was enriched using the hybrid capture (HC) probe set. The libraries included 191 DNA libraries and 192 RNA libraries, where to prepare\ some of the libraries 130 stool samples were each individually used to prepare both a DNA library and an RNA library (i.e., 130 DNA libraries and 130 RNA libraries prepared from each of the same respective 130 stool samples). The resulting libraries were then sequenced using Illumina technology. The mean DNA enrichment was 40-fold and the mean RNA enrichment was 23-fold.

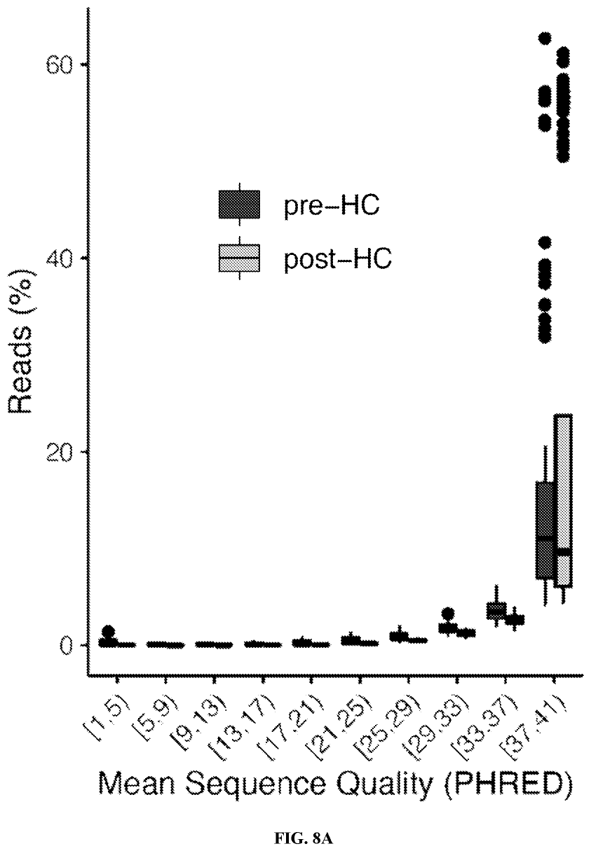

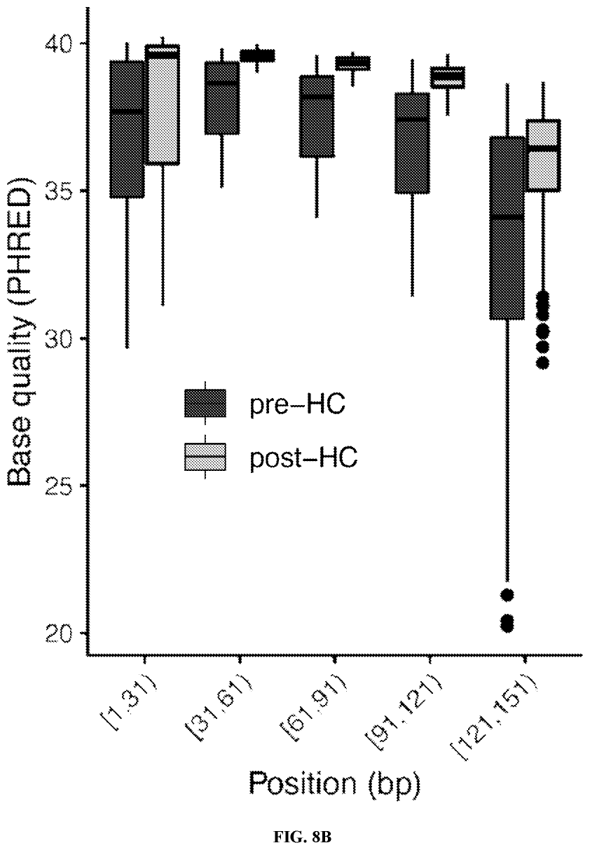

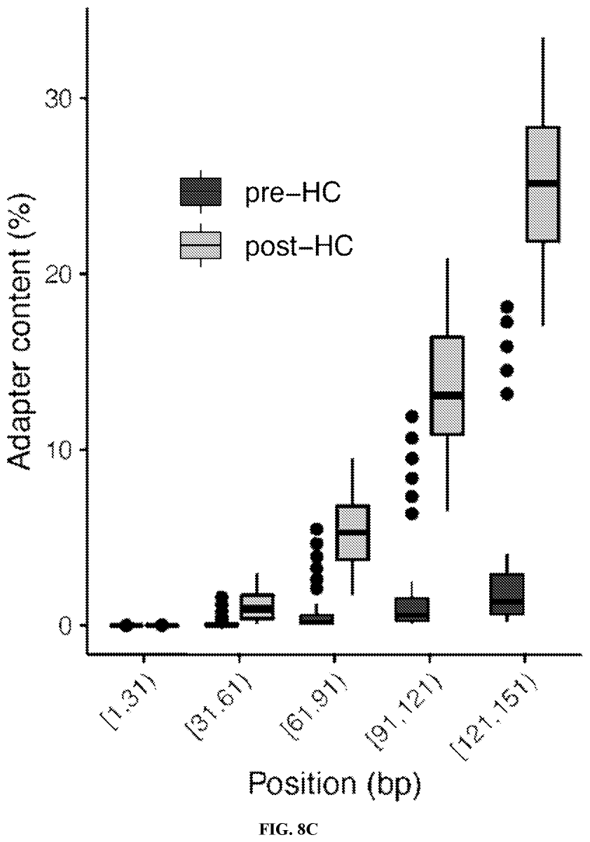

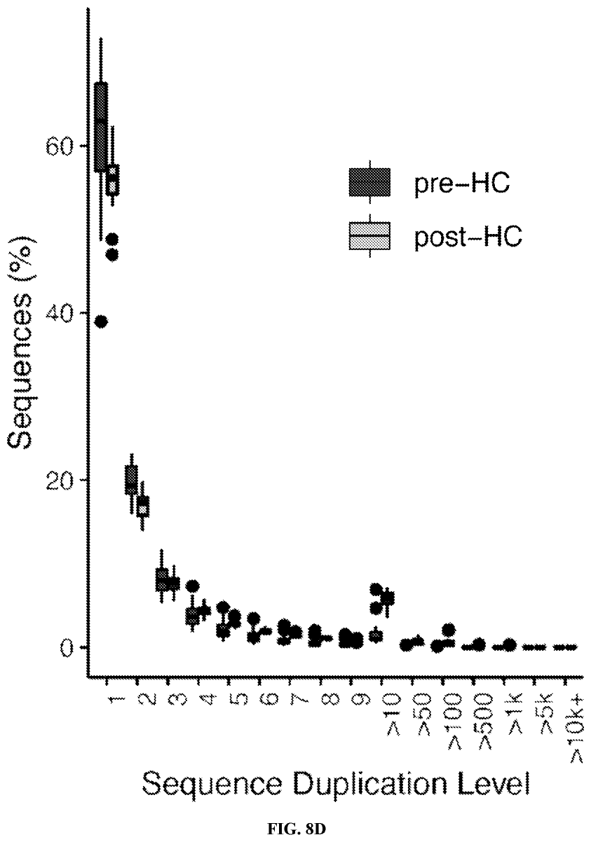

Sequence quality was assessed both pre- and post-hybrid capture (HC) to ascertain whether HC had a noticeable effect on the quality of sequencing data. Sequence and base quality metrics were slightly better for post-HC data (FIGS. 8A-8D). Adapter content for post-HC data was higher than pre-HC data (on average 0.7% vs. 6.8% pre-vs. post-HC, p<0.001, FIG. 8C), likely reflective of smaller insert sizes of the post-HC libraries, which was expected as Nextera Flex kit for enrichment, which was used to generate HC libraries, generates smaller insert sizes on average than the Nextera XT kit, which was used to generate pre-HC libraries. GC content was unaltered by HC (48.65% pre-HC vs. 48.60% post-HC, p>0.1). Taken together, these results suggest minimal impact on overall sequence quality due to HC.

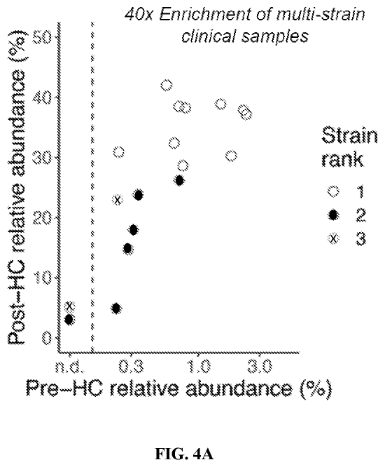

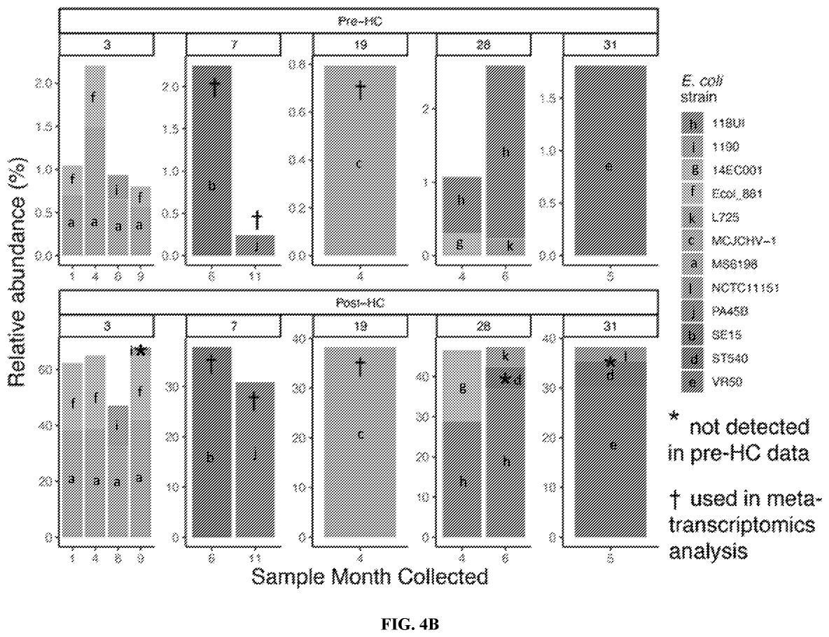

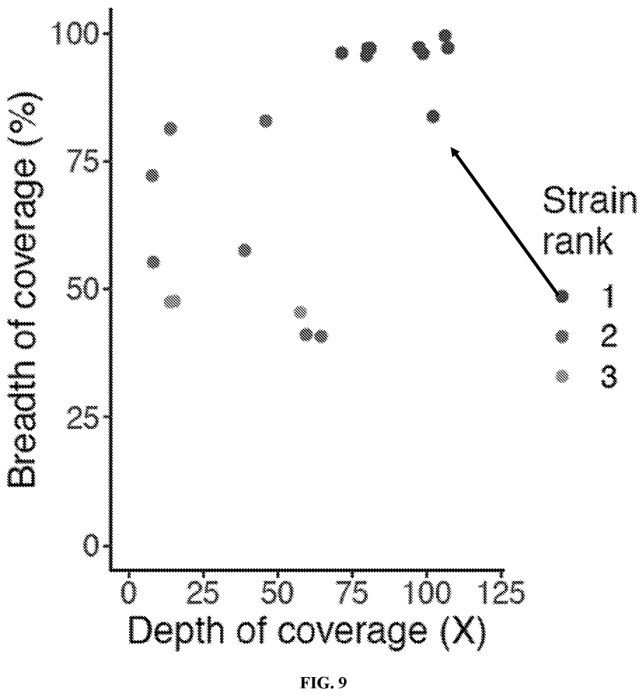

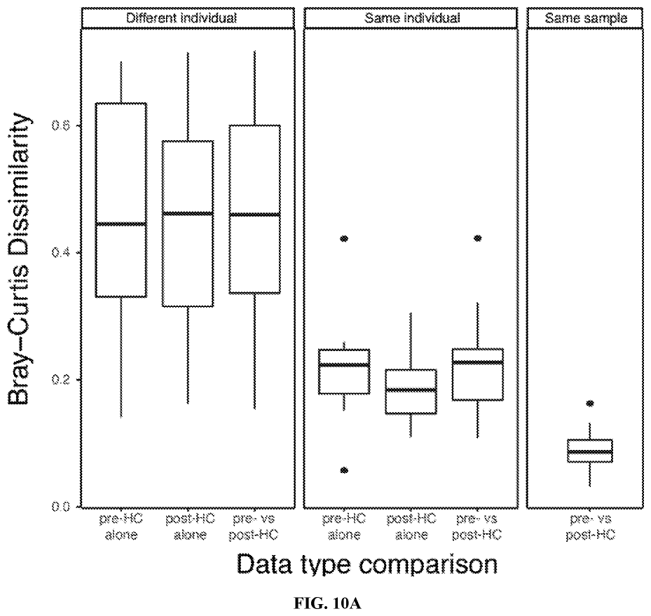

In order to assess whether different strains were enriched without bias after HC, strain composition of pre- and post-HC data was analyzed using StrainGE (Dijk, Lucas R. van, Bruce J. Walker, Timothy J. Straub, Colin J. Worby, Alexandra Grote, Henry L. Schreiber, Christine Anyansi, et al. 2021. “StrainGE: A Toolkit to Track and Characterize Low-Abundance Strains in Complex Microbial Communities.” Cold Spring Harbor Laboratory. doi.org/10.1101/2021.02.14.431013) (FIGS. 4A and 4B). As seen also in the mock community, E. coli relative abundance was dramatically increased after HC using multi-strain clinical samples, with an average log₂ fold-change of 5.2 (range 4.0 to 7.0), or about 40 times higher, post-HC vs. pre-HC (FIG. 4A). RNA was enriched by about 23-fold. Further, less abundant strains were more highly enriched, while more abundant strains were modestly enriched. This overall increase in relative abundance of E. coli led to high depth and breadth of coverage in post-HC data (FIG. 9), similar to what was seen for the mock mixture. Sequencing of the enriched DNA and RNA allowed for new strains (DNA sequence data) and transcripts (RNA sequence data) to be uncovered that were below the pre-hybrid capture detection threshold.

For the vast majority of samples, the same strains that were detected pre-HC were also detected post-hybrid capture (HC) (FIG. 4B), indicating that HC was unbiased in its enrichment. In both pre- and post-HC samples, the same strains were often observed across time points from the same participant. Notably, four additional E. coli strains were discovered in post-HC data (points to the left of the dashed vertical line in FIG. 4A; asterisks in FIG. 4B). One of these strains was also detected in a previous time point from the same participant. Overall, these results highlight the power and ability of HC to unearth low abundance strains that traditional methods would fail to detect.

Due to the dramatic increase in depth and breadth of coverage across the strain genomes (FIG. 9), metagenomic assembly of these samples was attempted to yield E. coli sequence assemblies, as done for the mock community. These assemblies produced varying amounts and quality of E. coli sequence, all being equivalent to at least one E. coli genome in size, with single-strain samples producing more contiguous assemblies than samples with multiple strains (Table 3). A binning method was then used to generate E. coli metagenome-assembled genomes (MAGs) with high levels of completeness and low levels of non-E. coli contamination, as determined by presence of Enterobacteriaceae CheckM marker genes, for all 10 samples (Table 4). A subset of these samples had higher levels of strain heterogeneity, and all of these samples had multiple strains of E. coli detected in the sample. However, there were samples with multiple strains that did not have any strain heterogeneity detectable by CheckM.

TABLE 3
E. coli metagenomic assembly metrics for post-HC stool samples

| Sample | Contig N50 | | Largest contig | | Total content | | Contigs** |
|---|---|---|---|---|---|---|---|
| 3.1 | 53 | kb | 197 | kb | 5.1 | Mb* | 216 |
| 3.4 | 123 | kb | 271 | kb | 5.2 | Mb* | 160 |
| 3.8 | 13 | kb | 41 | kb | 5.6 | Mb* | 1,325 |
| 3.9 | 17 | kb | 58 | kb | 5.5 | Mb* | 986 |
| 7.6 | 213 | kb | 540 | kb | 4.8 | Mb | 55 |
| 7.11 | 31 | kb | 103 | kb | 5.1 | Mb | 302 |
| 19.4 | 98 | kb | 239 | kb | 4.7 | Mb | 136 |
| 28.4 | 11 | kb | 40 | kb | 6.1 | Mb* | 1,494 |
| 28.6 | 10 | kb | 60 | kb | 5.8 | Mb* | 991 |
| 31.5 | 21 | kb | 116 | kb | 5.9 | Mb* | 978 |

*It is possible that more than one genome (i.e., >5 Mb, which is the approximate size of the E. coli genome) was assembled, as multiple strains were detected in the same
**Only including contigs >1 kb in length

TABLE 4
CheckM metrics for E. coli metagenome-assembled genomes (MAGs)
generated from post-hybrid capture (HC) stool samples

| Sample | Completeness | Contamination | Strain Heterogeneity |
|---|---|---|---|
| 3.1 | 99.67% | 0.16% | 0% |
| 3.4 | 99.97% | 0.16% | 0% |
| 3.8 | 77.2% | 0.14% | 0% |
| 3.9 | 86.82% | 0.59% | 55.56% |
| 7.6 | 99.97% | 0.07% | 0% |
| 7.11 | 96.71% | 0.09% | 0% |
| 19.4 | 99.67% | 0.65% | 0% |
| 28.4 | 76.59% | 0.55% | 62.5% |
| 28.6 | 93.24% | 0.26% | 50% |
| 31.5 | 95.38% | 0.33% | 0% |

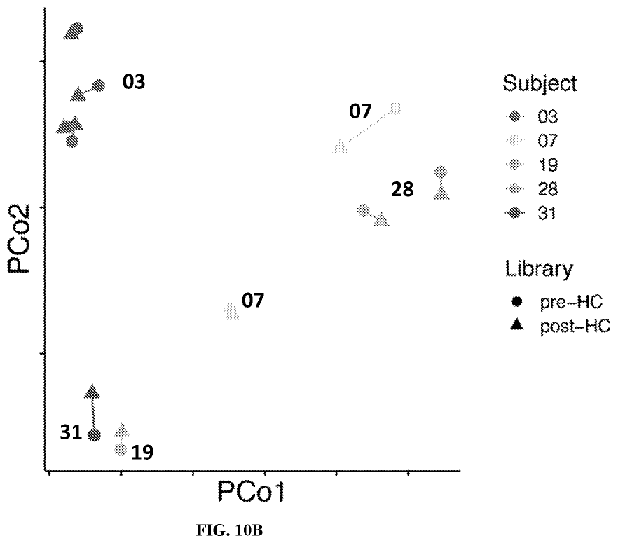

In order to assess the bias that HC may have introduced in the non-E. coli content of the metagenome, taxonomic composition of pre-vs. post-HC data was compared by removing E. coli taxa and recalculating relative abundances of the remaining taxa. Pairwise Bray-Curtis dissimilarity values were then calculated for all samples. Pre-vs. post-HC of the same sample were more similar than different samples from the same individual, which themselves were more similar than samples from different individuals (FIG. 10A). These results indicated that, if HC did introduce bias to non-E. coli content, it was less variable than comparing an individual's gut community from one month to another, and far less than comparing different subjects' microbiomes to one another. This pattern was borne out in principal coordinate analysis (PCoA) as tight clustering by sample and, to a lesser extent, subject, rather than by pre-vs. post-HC libraries (FIG. 10B).

### Example 4: Enrichment of E. coli Transcripts From Metatranscriptomic Libraries Derived From Human Stool Samples

The probe set was next used to enrich E. coli transcripts from RNA sequencing libraries derived from the same stool samples as discussed in Example 3. The first samples to be analyzed were three samples comprising only a single detected E. coli strain. These three samples, two of which were derived from different time points from the same patient, are indicated in FIG. 4B with a “†”. In order to assess overall level of enrichment, the closest reference genome for each of the three samples selected by StrainGST based on analysis of DNA metagenomic data (FIG. 4B) was used to align pre- and post-HC metatranscriptomic data. Selecting the closest reference was done to increase precision and the chances of detecting unique transcripts for each sample's strain.

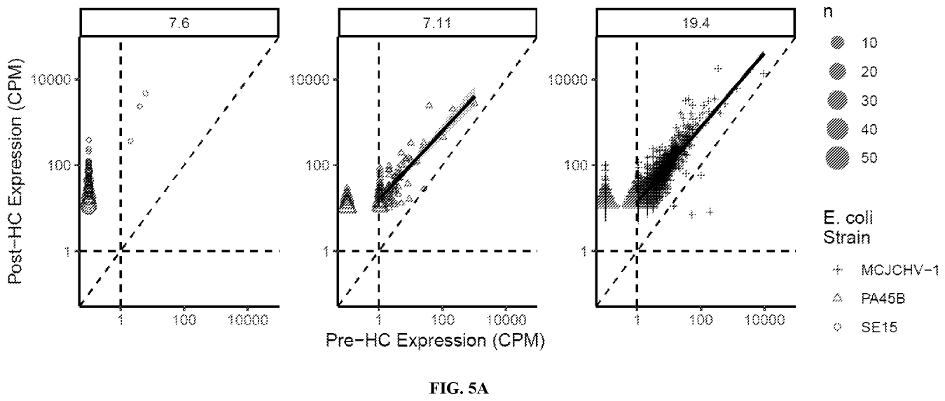

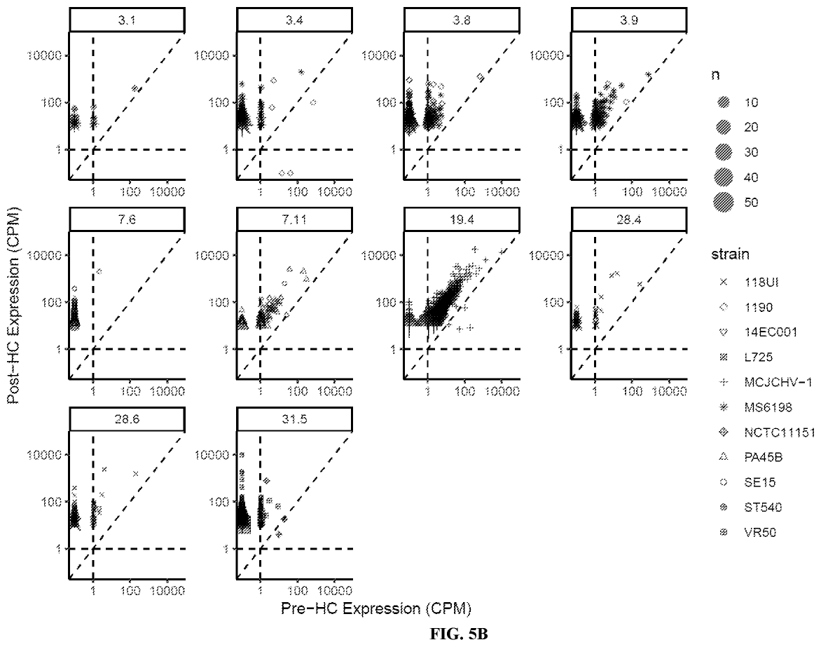

The percentage of aligned reads increased for all three samples, modestly so for two (7.11 and 19.4), both with 3.1 log2 fold-change post-HC, and dramatically so for one (7.6), with a 10.2 log2 fold-change post-HC (348 aligned reads pre-HC [0.003%] and 181,372 aligned reads post-HC [3.4%]). These increases in aligned reads also led to increases in both the number of unique transcripts detected and an overall increase in normalized expression for almost all transcripts in the post-HC data (FIG. 5A). For the two samples where there was sufficient pre-HC expression for detection and analysis, post-HC expression was highly and significantly correlated (log-transformed data, R²=0.74, p<10-15) for transcripts that were detected (≥10 CPM) in both pre- and post-HC data (FIG. 5A).

The analysis was next expanded to all RNA samples, including those with more than one E. coli strain. Reads were aligned to the concatenated reference of all strains found in each individual (whether or not they were present at a given time point/sample). The percentage of reads aligning to the reference was increased post-HC in all samples, with a mean log₂ fold-change of 4.5 (range 3 to 10.2), or about 23 times higher percent aligned reads post-HC. Due to this successful enrichment, on average 167 (range of 28-366, total of 1,472 unique transcripts across all samples) transcripts per sample were detected post-HC that were not detected pre-HC (FIG. 5B). In contrast, there was minimal transcript drop-out due to HC. Two unique transcripts in one sample (3.4) were detected exclusively in pre-HC data, both of which were multispecies proteins common to Enterobacteriaceae encoded on the same plasmid found in the E. coli 1190 reference.

### Discussion

Here, it has been demonstrated that the pan-genome-based probe set, which was designed to capture the immense diversity of E. coli, successfully enriched both DNA and RNA sequences from known mixtures and clinical samples, both containing mixes of diverse E. coli strains, in an unbiased fashion. This enrichment allowed for near complete observations of all four known strains of E. coli from a mock mixture, as well as for detection of novel strains in human stool samples that were below the limit of detection pre-HC.

Further, this enrichment was relatively unbiased, as the diverse set of strains detected pre-HC were also detected, at higher abundance, post-HC. It was further determined that the non-E. coli content of the metagenome was also minimally biased. This suggests that one may be able to perform HC on a sample and not also have to sequence a pre-hybrid capture (HC) library, potentially saving time, money, and precious sample material, while thoroughly investigating the E. coli content of the sample.

This enrichment of E. coli allowed for a wide range of analyses that were previously impractical and cost-prohibitive due to the required amount of deep sequencing. Large contigs of E. coli sequence were successfully assembled from post-HC data, suggesting that near complete E. coli references may be obtained from clinical samples without the need for culture. Tools that specifically attempt to disentangle strain resolution in metagenomic assemblies, such as DESMAN (Quince, Christopher, Tom O. Delmont, Sébastien Raguideau, Johannes Alneberg, Aaron E. Darling, Gavin Collins, and A. Murat Eren. 2017. “DESMAN: A New Tool for de Novo Extraction of Strains from Metagenomes.” Genome Biology 18 (1): 181) and STRONG (Quince, Christopher, Sergey Nurk, Sebastien Raguideau, Robert James, Orkun S. Soyer, J. Kimberly Summers, Antoine Limasset, A. Murat Eren, Rayan Chikhi, and Aaron E. Darling. 2020. “Metagenomics Strain Resolution on Assembly Graphs.” Cold Spring Harbor Laboratory. doi.org/10.1101/2020.09.06.284828), may be useful for robust assembly of multiple strains.

Hybrid capture (HC) also enriched E. coli content from metatranscriptomic libraries in an unbiased manner. 1,608 unique transcripts were recovered in post-HC data that were not detected pre-HC. Thus, hybrid capture (HC) can provide the sensitivity required to analyze and compare otherwise cryptic gene expression patterns of low-abundance E. coli in the human gut.

This probe set can provide a valuable tool to push forward the study and understanding of E. coli in metagenomic contexts, including the gut niche. Understanding how commensal and pathogenic E. coli strains differ in their gene content and expression, how E. coli responds to antibiotics, and the dynamics of E. coli over time and disease in vivo can uncover exciting biology and lead to alternative therapeutic options for diseases caused by E. coli. Further applications outside the gut include detection of E. coli in other specimens and environments in which it would be of low abundance, including the study of other body sites such as the skin or bladder, or even applications such as pathogen surveillance at hospitals and in the food industry.

The exclusion of a gene from probe design does not necessarily mean that the probe set won't capture the gene, as they could still share enough sequence identity to, or could be found in close enough physical proximity to genes that were included in probe design. Further, data showed that the hybrid capture probes covered some genes and orthogroups that were not specifically part of the design set, so genes may be enriched from genomes not used in probe design.

The probes can be suitable for use in long read sequencing technologies, such as those discussed in Bertrand, Denis, Jim Shaw, Manesh Kalathiyappan, Amanda Hui Qi Ng, M. Senthil Kumar, Chenhao Li, Mirta Dvornicic, et al. 2019. “Hybrid Metagenomic Assembly Enables High-Resolution Analysis of Resistance Determinants and Mobile Elements in Human Microbiomes.” Nature Biotechnology 37 (8): 937-44. The probes can be applied in some embodiments to long, native strands of metagenomic DNA. A combination of hybrid-captured short-read sequencing and native long-read sequencing may be useful for metagenomic assembly of low-abundant organisms.

This hybrid capture methodology represents a cost-effective alternative to ultra-deep metagenomic sequencing to examine low-abundance species. Though E. coli was chosen for this study, this pan-genome-based approach for probe design can also be suitable for other sequenced bacteria, including, as a non-limiting example, those that are: 1) at low abundance in their environment; 2) well-studied with numerous representative reference genomes; and/or 3) genetically complex and diverse.

Computationally designed hybrid capture (HC) probes targeting a pan-genome provide a promising method to enrich sequences derived from low-abundance, highly diverse species in complex metagenomic and metatranscriptomic communities. Using this method, E. coli sequence was successfully enriched from both a mock mixture of four diverse strains of E. coli in 99% human background, as well as from human stool samples. E. coli was enriched, on average, 40-fold (for DNA) and 23-fold (for RNA) post-hybrid capture. This probe set, and the hybrid capture methods described herein, will be useful to many applications where researchers and clinicians are interested in low-abundance but diverse microbes, such as E. coli.

### Example 5: Probe-Based Enrichment Greatly Increased Coverage of the E. coli Pan-Genome in Metagenomic Sequencing Data Exposing Regulation of a Clinically Relevant Urovirulence Factor from Gut Meta'omes

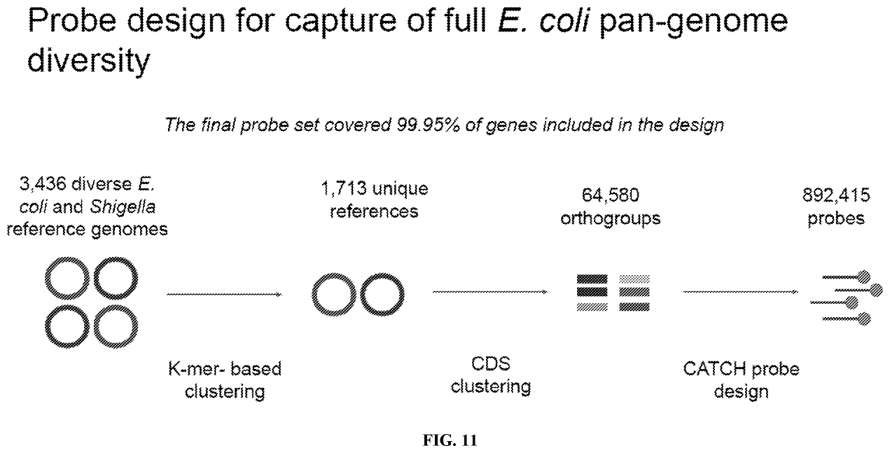

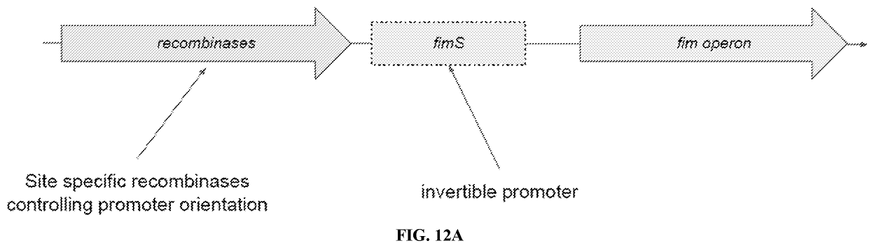

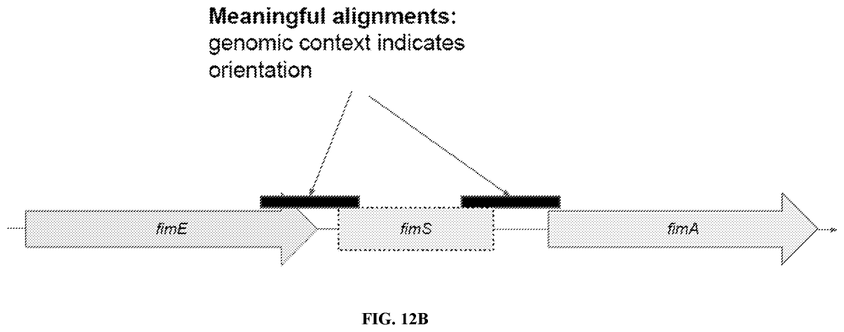

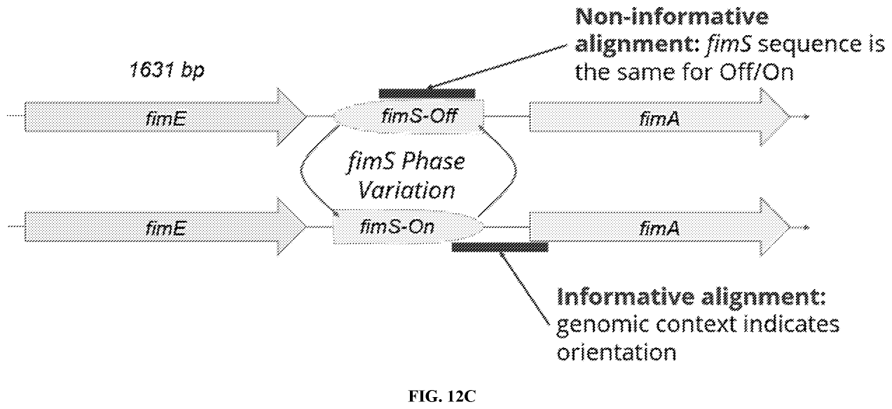

Clinically important microbes are often found at low abundances (<1%) within complex communities, presenting a challenge for their investigation. In order to gain a high-resolution view of one such gut-associated bacterial species, a set of hybrid capture probes was designed, as described above, to represent the more than 8 million genes of the Escherichia coli pangenome (FIG. 11). The approach enriched E. coli sequence from stool DNA libraries by an average of approximately 40-fold, and from stool RNA libraries by approximately 23-fold with little to no bias in the breadth or depth of sequencing coverage. As a demonstration of the enhanced resolution provided by this approach, experiments were undertaken to evaluate the expression of the fim operon encoding Type 1 fimbriae (also known as “pili”), which is an important urovirulence factor having a role in invasion of bladder urothelial cells in urinary tract infections (UTIs) and is regulated by an invertible promoter, fimS. Type 1 fimbriae are hair-like fimbriae used by E. coli to attach to uroepithelial cells. Increased expression of Type 1 fimbriae in the guts of subjects with recurrent urinary tract infections (rUTIs) could be a contributing factor to increased UTI frequency. Type 1 fimbriae allow for E. coli to bind epithelial cells of a host urinary tract and are upregulated in isolates from urinary tract infections. Using data from libraries enriched with the E. coli pangenome probes, estimates were successfully made of the proportion of fimS in the “on” orientation within the gut microbiota of women with and without a history of recurrent UTI, as well as a recapitulation of the established dependence of fim operon expression on fimS orientation. The fimS orientation (FIGS. 12A-12C) can be observed through alignments to boundary regions. These experiments, which are detailed further below, demonstrated the power of the E. coli hybrid capture methodology to yield unprecedented insight into E. coli dynamics within real world contexts where the genetics of the E. coli are unknown, and their relative abundances are low.

The genomic region for the fim operon promoter is shown in FIG. 12A and FIG. 12C provides a schematic showing fimS phase variation (i.e., inversion of a region of the fim operon promoter to the on and off configurations). The fim operon promoter (fimS) is invertible, where the inversion is associated with recombinases encoded upstream of the fim operon. The fimS orientation can be determined through alignments to the boundary region of fimS, which does not undergo inversions. To be meaningful, such alignments should span both a portion of fimS and a portion of a surrounding region (FIG. 12B). On account of challenges presented by deep metagenome sequence, variation in gut microbiome fimS orientation was largely unknown previously.

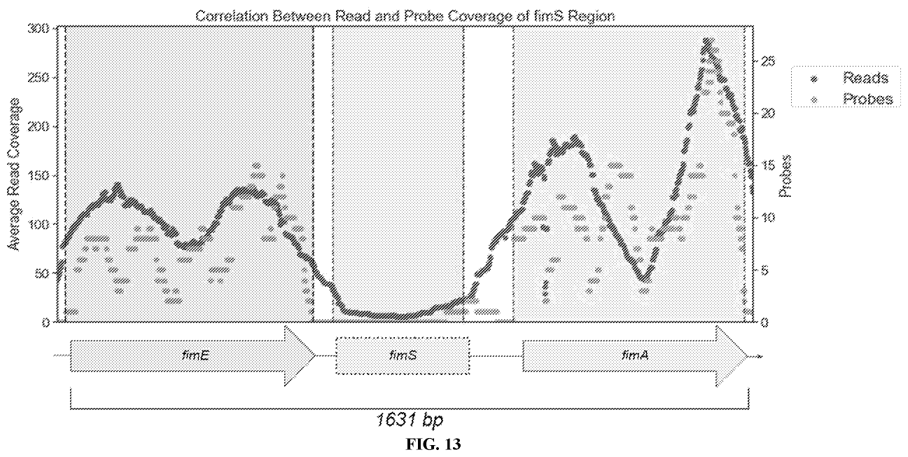

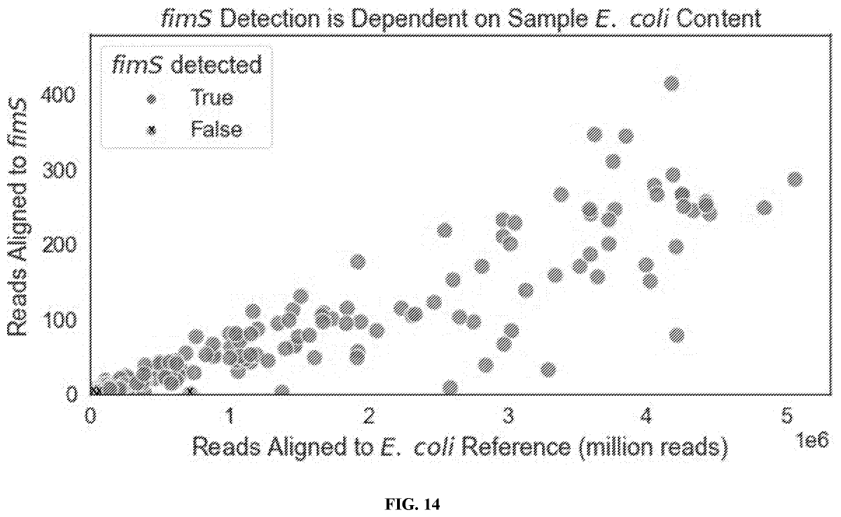

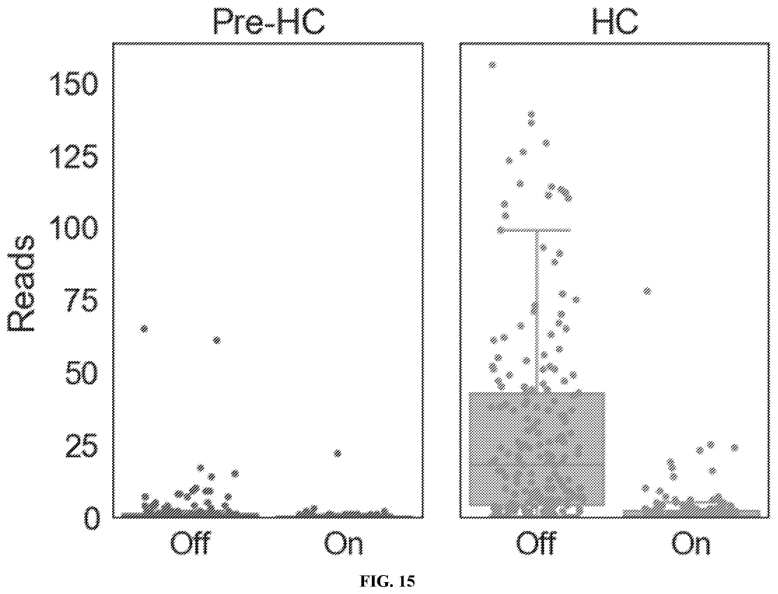

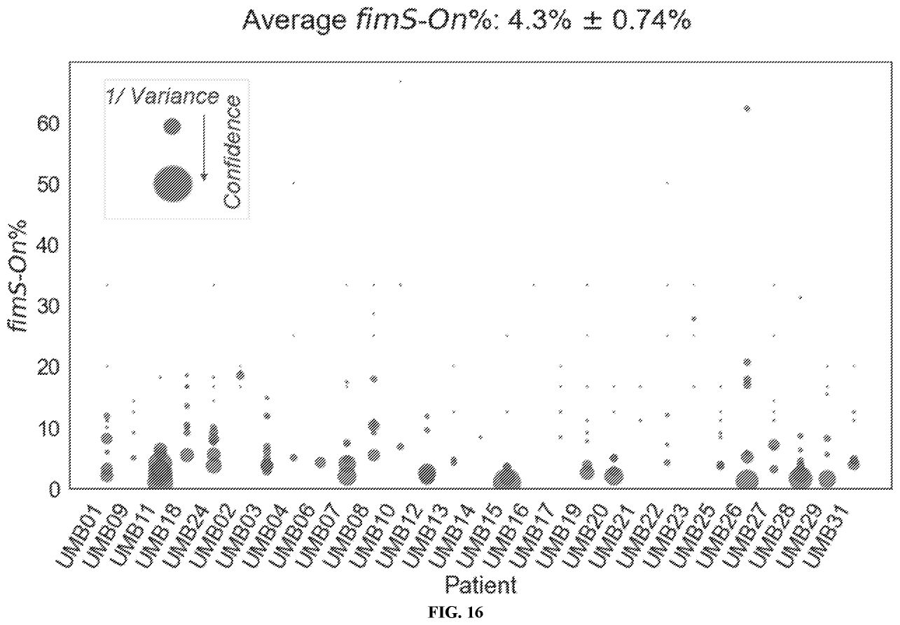

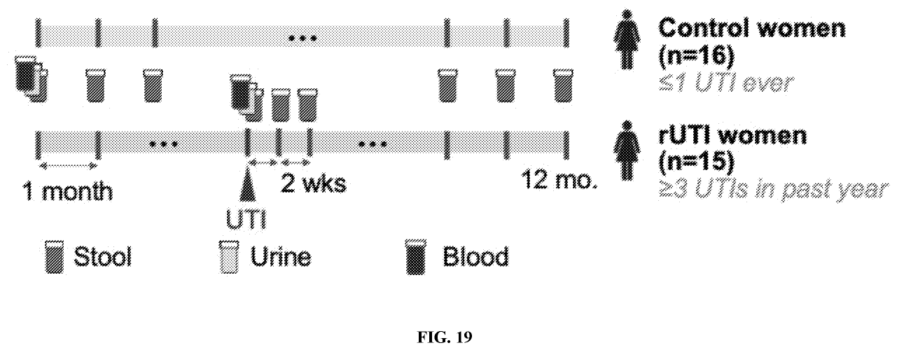

The E. coli pangenome was sequenced and analyzed for stool samples collected from participants in the Urinary Tract Infection Microbiome (UMB) Project (see Worby, C. J., Schreiber, H. L., Straub, T. J. et al. Longitudinal multi-omics analyses link gut microbiome dysbiosis with recurrent urinary tract infections in women. Nat Microbiol 7, 630-639 (2022)) (FIG. 19). The hybrid capture probes successfully enriched boundary regions of fimS (FIG. 13). Hybrid capture sequencing indicated that the fim operon was carried by almost all subjects participating in the UMB project having gut E. coli (FIG. 14). The fimS reads were detected in 178 out of 191 enriched samples. Samples lacking fimS had extremely low abundances of E. coli. Hybrid capture provided about 10× enrichment of both inversion phases of the fimS promoter (FIG. 15). Only 65 pre-hybrid capture samples had a read aligning to fimS. In the UMB dataset, the average sample had 4.3% fimS activation (from weighted least squared: 4.3% +/−0.74%; n=178) (FIG. 16). There was high inter-patient and inter-sample variability of fimS activation (11/178 samples had fimS-ON%>10% (α=0.05). No significant difference in activation was found between cohorts (i.e., a cohort of women with a history of rUTI and a cohort of matched healthy women) despite significant intra-individual variation. In this analysis, fimS' was overwhelmingly in the “off” configuration in the rUTI cohort stool metagenomes (103/178 samples lacked fimS-O) N reads). Therefore, hybrid capture led to an increase in fimS coverage of about 14× relative to non-enriched samples and allowed for analysis of the regulation of Type 1 fimbriae in stool meta'omes at high resolution.

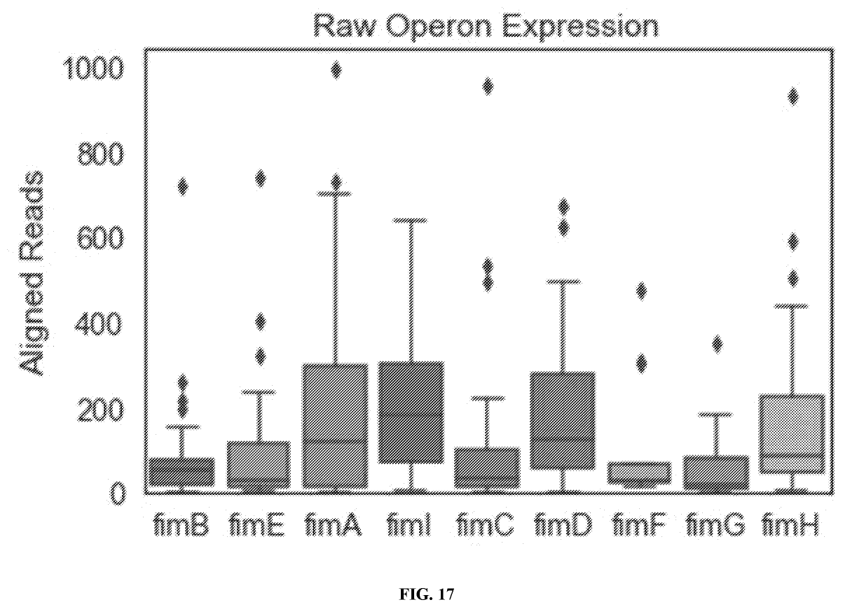

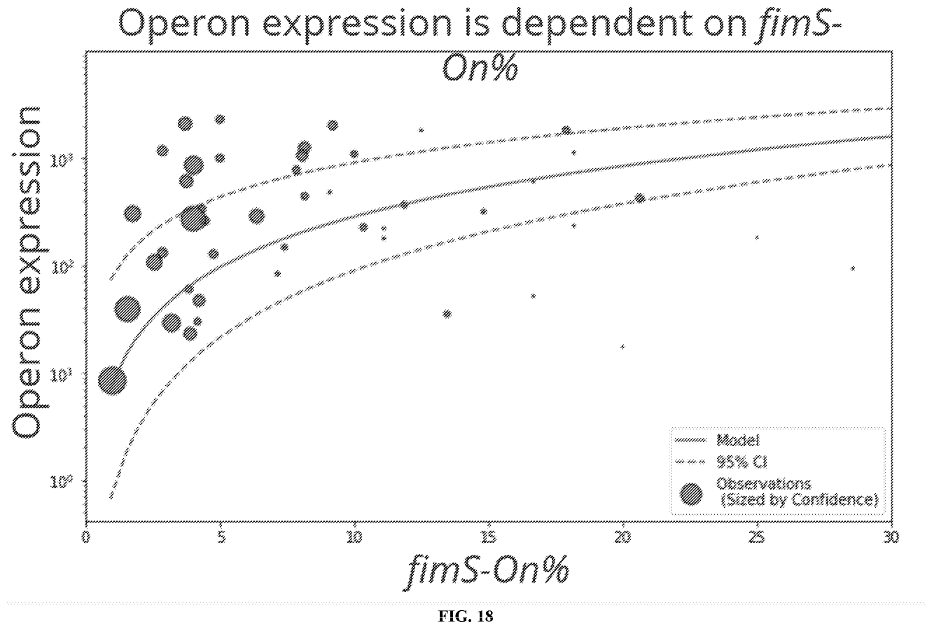

Paired RNA hybrid capture samples allowed for analysis of fim operon expression (FIG. 17). Sequencing of RNA enriched using RNA hybrid capture revealed low expression of the fim operon. The average coverage of the protein-coding region of the operon (fimA-fimH) was 138 reads. Samples with lower abundances of E. coli were filtered out prior to analysis using the following filter: 80,000 reads aligning to an E. coli reference genome; E. coli detected in DNA and RNA to a depth of greater than 80 k (71 out of 130 samples met this depth criterion). Sequence data prepared using samples enriched using hybrid capture recapitulated the expected relationship between fimS orientation and operon expression (p<1e-8) (FIG. 18). Operon expression was significantly dependent on operon orientation (i.e., fimS-ON%). Operon expression was determined by aligning RNA sequence reads to the protein coding region of the fim operon and normalizing by sample E. coli content. This relationship was not detected using non-enriched samples.

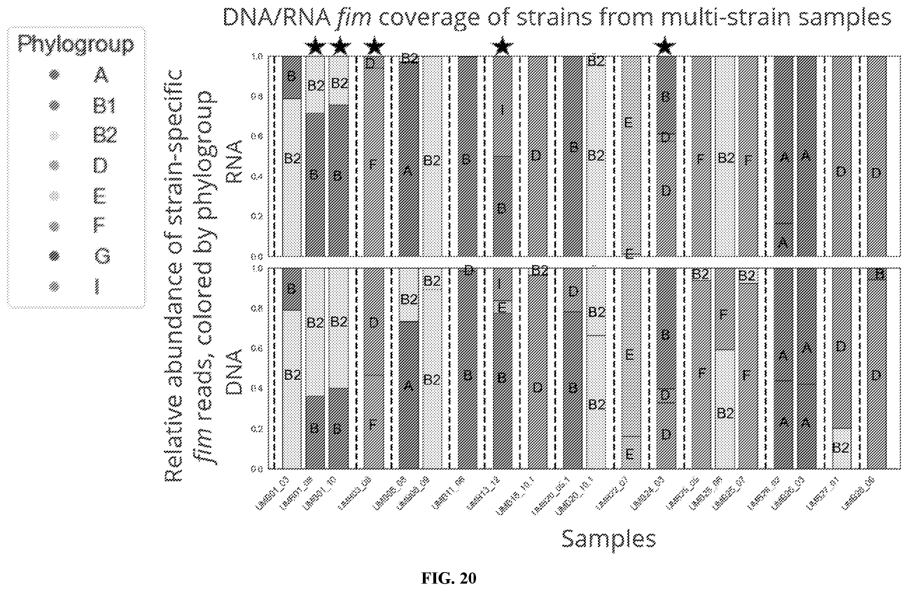

Analysis of sequence data prepared using samples enriched using hybrid capture revealed heterogeneity of strain-specific fim expression within multi-strain samples. For strain deconvolution, strain-specific single-nucleotide polymorphisms (SNPs) were detected using the StrainGE suite (van Dijk, L. R., et al., “StrainGE: a toolkit to track and characterize low-abundance strains in complex microbial communities,”0 Genome Biol 23, 74 (2022). doi: 10.1186/s13059-022-02030-0) (FIG. 20). On average >70% fim-aligning DNA and RNA reads per multi-strain sample could be assigned to a strain using strain-specific SNPs.

Hybrid capture enabled the study of expression and regulation from a metagenomic context. Using the hybrid capture probes, E. coli DNA was enriched by about 40-fold and E. coli RNA was enriched by about 23-fold. Enrichment increased coverage of genomic sites that were up to 450 bp away from a probe-binding site. By sequencing DNA and RNA samples collected from participants in the Urinary Tract Infection Microbiome (UMB) Project enriched using the hybrid-capture probes, it was determined that at least about 3% of fimS promoters detected were in the “on” configuration and that there was a detectable correlation between fimS orientation and fim operon expression.

### METHODS OF THE EXAMPLES

The following methods were employed in the above examples.

### Probe Design

In June 2017, all 295 E. coli and Shigella (herein collectively referred to as E. coli) complete genomes were downloaded from NCBI RefSeq. In addition, to obtain a larger diverse collection of references, 3,141 publicly available, high quality (L50<20) genomes of E. coli that were listed in the NCBI Pathogen Detection database were downloaded from GenBank from July to August 2017 (Table 4). Of these, 136 Genbank genomes were redundant with (i.e., identical) RefSeq complete genomes.

In order to remove redundant and nearly identical genomes, k-mer based clustering was performed. All 3,141 Genbank genomes were k-merized (using 23-mers) with the StrainGST “kmerize” tool from StrainGE (Dijk, Lucas R. van, Bruce J. Walker, Timothy J. Straub, Colin J. Worby, Alexandra Grote, Henry L. Schreiber, Christine Anyansi, et al. 2021. “StrainGE: A Toolkit to Track and Characterize Low-Abundance Strains in Complex Microbial Communities.” (Cold Spring Harbor Laboratory. doi.org/10.1101/2021.02.14.431013), then their pairwise all-vs-all Jaccard similarities were calculated. Single-linkage clustering was performed on these similarities at a 95% threshold to construct genome clusters. This generated 1,485 clusters, of which 1,124 contained a single genome and 361 contained two or more Genbank genomes.

The GenBank accession numbers for the 3,141 genomes (“the 3, 141 GenBank genomes”) were the following, where the identifier “GCA” (i.e., the designation as GenBank assemblies) that precedes each number has been omitted: 000462285.2; 000249095.2; 000447145.2; 000987875.1; 000778215.1; 000460875.1; 000753515.1; 000407725.1; 001284285.1; 000617245.2; 001269145.1; 000782055.1; 000223035.2; 001059155.1; 001286085.1; 000408605.1; 000776065.1; 000458155.1; 000800215.1; 000768465.1; 000235205.1; 001419885.1; 000713745.1; 000303615.2; 000352265.1; 000351705.1; 000776285.1; 000781715.1; 000459335.1; 000350825.1; 000778055.1; 001281775.1; 000618545.1; 001467005.1; 000713345.1; 000250175.2; 001266435.1; 000316885.2; 000601315.1; 000172015.1; 000819205.1; 000357025.2; 000948055.1; 000494995.1; 000355435.2; 000249815.2; 000326225.1; 000752975.1; 000617765.2; 000711375.1; 000700265.1; 000522325.1; 000617725.2; 000267305.2; 000459135.1; 000785765.1; 000457955.1; 000714385.1; 001446595.1; 001283185.1; 000778235.1; 000704945.1; 000467695.1; 000249355.2; 001266005.1; 001463405.1; 000241995.1; 000614595.1; 001561105.1; 000459795.1; 000352185.1; 000356585.2; 001265655.1; 000714145.1; 000703625.1; 000777815.1; 001012275.1; 000461035.1; 000779995.1; 000258865.1; 001281725.1; 000778015.1; 001284845.1; 001268625.1; 000798055.1; 001277475.1; 000460175.1; 000267865.2; 001309475.1; 000647495.1; 000350745.1; 000618125.1; 000704265.1; 000351865.1; 000351905.1; 000780335.1; 000350005.1; 000703605.1; 000258615.1; 000303995.2; 000358355.2; 001283465.1; 000812715.1; 000418595.1; 000974535.1; 000194705.2; 001262855.1; 000781995.1; 000714135.1; 000777095.1; 000781935.1; 000779675.1; 000618145.2; 001012495.1; 001561285.1; 001284865.1; 000704065.1; 000778895.1; 000356905.1; 000193975.2; 000940035.2; 000459015.1; 001010165.1; 000354735.2; 000352405.1; 000711365.1; 000617565.1; 000356545.1; 000615385.1; 000618785.1; 000320175.1; 001266445.1; 000225205.2; 000753035.1; 000781435.1; 000941935.1; 000711455.1; 000692795.1; 001419845.1; 000622445.2; 000326885.1; 001562375.1; 000350925.1; 000335075.2; 000692815.1; 000357885.2; 000779025.1; 000814525.1; 000780655.1; 000446845.2; 000302715.1; 000247665.4; 001277515.1; 001262865.1; 000458995.1; 000194575.2; 000474825.1; 001284085.1; 000617625.2; 000616405.2; 000498255.1; 000462885.2; 000303855.2; 000622265.1; 000713035.1; 000358495.2; 000714105.1; 000249755.2; 001285765.1; 000704105.1; 000522085.1; 000782815.1; 000617745.1; 000459075.1; 000687085.1; 000184765.2; 000352585.1; 000619545.2; 000458115.1; 000281775.1; 000164495.1; 000690815.1; 000351825.1; 000827105.1; 000462405.2; 001264195.1; 000471245.1; 001485455.1; 000937095.2; 000457285.1; 000782375.1; 000446135.2; 000350725.1; 000350965.1; 000703245.1; 000354975.1; 001268405.1; 000462905.2; 000780835.1; 001265155.1; 000614305.2; 001286185.1; 001281965.1; 000458495.1; 000647455.1; 000462505.2; 000408045.1; 000776255.1; 000249455.2; 000459155.1; 000249775.2; 000224395.2; 000397465.1; 000457555.1; 001191265.1; 000713135.1; 000316445.2; 000622735.2; 000261405.1; 001283585.1; 000945615.2; 000407785.1; 000780435.1; 000779745.1; 000798075.1; 000461635.1; 000233675.2; 000458665.1; 000456925.1; 000768415.1; 000692575.1; 001268785.1; 001191375.1; 000026325.2; 000752835.1; 000615015.1; 000352065.1; 000713685.1; 000303255.2; 000713985.1; 000457225.1; 000171915.1; 001186315.2; 000777135.1; 000176635.2; 000647795.2; 000234255.3; 000352865.1; 000356505.2; 001550005.1; 001265575.1; 000025165.1; 000350025.2; 000462365.2; 000461275.1; 000460595.1; 000797715.1; 000460555.1; 000326725.1; 000714065.1; 000704445.1; 000714595.1; 000461715.1; 000494935.1; 000335135.1; 000352505.1; 001281985.1; 000703265.1; 000460395.1; 000777435.1; 000488555.1; 000215245.2; 001052755.1; 001262935.1; 000619565.2; 000780215.1; 000326525.1; 000703905.1; 000462745.2; 000211395.2; 000461435.1; 001561255.1; 000700185.1; 000619245.1; 001309815.1; 000456265.1; 000358085.2; 000616075.2; 000601155.1; 000616285.1; 000743255.1; 000599665.1; 000974865.1; 001282225.1; 000462105.2; 000351765.1; 001520015.1; 000779255.1; 000351345.1; 000010245.1; 000326945.1; 001309615.1; 000249835.2; 000193995.2; 000355415.2; 000776315.1; 000458355.1; 000235145.1; 000779615.1; 000249135.2; 001282215.1; 001462975.1; 000778075.1; 000356565.1; 001561555.1; 000779095.1; 000632615.2; 000797855.1; 000354855.1; 000458725.1; 000459455.1; 000776655.1; 000456165.1; 000614845.2; 001420065.1; 000408625.1; 000618325.1; 000511565.1; 000178315.1; 001266295.1; 001065165.1; 000937855.1; 000601275.1; 001262775.1; 000295775.2; 000357225.2; 000303715.2; 000778745.1; 000358995.1; 001561375.1; 000460735.1; 000704205.1; 000619125.2; 000326205.1; 000488075.1; 000166615.2; 000351445.1; 000692895.1; 000752735.1; 000618085.1; 001268925.1; 000397245.1; 000304095.2; 000692735.1; 000461475.1; 001012335.1; 000460195.1; 000358555.1; 000357825.2; 000356965.1; 000250335.2; 000456065.1; 000316565.2; 000250515.2; 001561515.1; 000617425.1; 001285565.1; 000704045.1; 000778545.1; 000457775.1; 000782435.1; 000321145.1; 000482065.1; 001519315.1; 000356045.2; 001281685.1; 000408305.1; 000267105.1; 000982435.1; 001519675.1; 000627945.1; 000503635.1; 000460335.1; 000944015.1; 000263895.1; 001413455.1; 000778275.1; 000522205.1; 000215165.1; 000354495.2; 000700225.1; 000753215.1; 000026345.1; 000495095.1; 000948785.1; 001268345.1; 001542675.2; 001284725.1; 000614255.1; 000350845.1; 001561835.1; 000627315.1; 001561785.1; 000617605.2; 001191315.1; 001012085.1; 000459435.1; 000773475.1; 000615175.1; 000978845.1; 000313445.1; 000356805.2; 000461675.1; 000250495.2; 000326545.1; 000164375.1; 000714975.1; 000619365.1; 000176555.2; 000619445.1; 000026245.1; 000780895.1; 000358735.1; 000798155.1; 000616705.2; 001277535.1; 001268365.1; 000303835.2; 000358855.2; 001077945.1; 001286665.1; 000488095.1; 000599685.1; 000981485.1; 000617145.2; 000819245.1; 000446325.2; 000781895.1; 000517245.1; 000456025.1; 000194555.2; 001268585.1; 000522265.1; 001191205.1; 000473725.2; 001419825.1; 000352905.1; 000267185.1; 001039225.1; 000488055.1; 000618065.2; 000951745.1; 000780555.1; 000460155.1; 001284065.1; 000752355.1; 000354815.2; 000462565.2; 000356685.2; 001285825.1; 000178695.1; 001519715.1; 000495075.1; 001297985.1; 000249535.2; 000782035.1; 000618945.2; 001413745.1; 000358615.2; 000692475.1; 000457125.1; 000460495.1; 000194495.2; 000355555.2; 001306605.1; 000785795.1; 000210475.1; 000387865.2; 001062415.1; 000695505.1; 000459195.1; 001284005.1; 000397445.1; 001269065.1; 000459175.1; 000351625.1; 001519875.1; 000752535.1; 000026265.1; 001520775.1; 000711435.1; 000782155.1; 001268705.1; 000459815.1; 000457185.1; 000797655.1; 000458315.1; 000303935.2; 001268905.1; 000953515.1; 001269265.1; 000503655.1; 000356705.1; 000447085.2; 000752875.1; 000778765.1; 000687125.1; 000522305.1; 000753135.1; 000703925.1; 000511505.1; 001519285.1; 000460615.1; 000496995.1; 000354935.2; 001561015.1; 000459515.1; 001012105.1; 001286125.1; 001413875.1; 000616245.1; 000167875.1; 000356005.2; 000974465.1; 000176595.2; 000781695.1; 001413685.1; 000703565.1; 000777675.1; 000461735.1; 001561275.1; 000171975.1; 000457815.1; 000194235.2; 000335215.2; 000355515.2; 000456385.1; 001064885.1; 001561575.1; 000303635.2; 000351185.1; 000458915.1; 001309455.1; 000627855.1; 001268825.1; 000235185.1; 001056975.1; 000352345.1; 000267385.2; 000462485.2; 000601295.1; 000618025.1; 000695135.1; 000194475.2; 001286105.1; 000614515.1; 000460035.1; 000776965.1; 000326645.1; 000331615.1; 000781635.1; 001268485.1; 000352565.1; 000446345.2; 000780295.1; 000713425.1; 000356745.2; 000627175.1; 000777535.1; 000782015.1; 001269045.1; 000493755.1; 000779545.1; 000457695.1; 001561135.1; 000461315.1; 001191215.1; 000352125.1; 000458875.1; 001284025.1; 000701105.2; 000407665.1; 000507665.1; 000447065.2; 000267765.2; 000780135.1; 000616305.2; 000316345.2; 000457935.1; 000704405.1; 000780235.1; 000357985.2; 000326145.1; 000768485.1; 000488435.1; 001268525.1; 000462425.2; 000458035.1; 000460135.1; 000250295.2; 000777845.1; 000495015.1; 000462165.2; 000462645.2; 000459315.1; 000776235.1; 000777735.1; 001519115.1; 000777695.1; 000335415.2; 000352925.1; 001282345.1; 001285125.1; 000351305.1; 000458055.1; 001266335.1; 000619745.2; 000488475.1; 000617965.1; 000700105.1; 000703645.1; 001283965.1; 000267925.2; 000326485.1; 000457515.1; 000616135.2; 000352785.1; 000777475.1; 000357765.2; 000355235.2; 000798575.1; 000714225.1; 000358935.2; 001413885.1; 000326705.1; 000333195.1; 001268865.1; 000456485.1; 000511445.1; 001420165.1; 000616545.2; 000461095.1; 001297725.1; 001055685.1; 000782715.1; 000617265.1; 000326805.1; 000617685.1; 000941895.1; 000407805.1; 000749565.1; 000258145.1; 000407985.1; 000190995.1; 001077935.1; 000304135.2; 000627925.1; 001419925.1; 000617445.2; 000465135.1; 000780395.1; 001419965.1; 000303735.2; 000615695.1; 000619145.1; 001282375.1; 000939195.1; 000781375.1; 000619005.2; 000181775.1; 000304875.2; 000181735.1; 001544635.1; 001056665.1; 000357845.2; 000357665.2; 000194725.2; 001012075.1; 000777325.1; 000616465.2; 001283385.1; 000692615.1; 001012195.1; 000967155.1; 000176535.2; 000831565.1; 000175735.1; 000358375.1; 000713935.1; 000456525.1; 000458175.1; 000305395.2; 000397285.1; 000022225.1; 000316405.2; 001441335.1; 001277415.1; 000459595.1; 001011995.1; 000357705.2; 000264095.1; 000779795.1; 000250395.2; 000807565.1; 000250555.2; 000687045.1; 000303375.2; 000692855.1; 000215205.2; 000617485.2; 000397705.1; 001262965.1; 001265795.1; 000446525.2; 000818995.1; 000408245.1; 000326665.1; 000407965.1; 000408365.1; 001562755.1; 000447005.2; 000356025.2; 001448025.1; 000507685.1; 001285445.1; 001309735.1; 000461395.1; 000700625.1; 000939755.1; 001520875.1; 000714015.1; 000830035.1; 000462525.2; 001056895.1; 000618825.1; 000703345.1; 000357345.2; 000713605.1; 000714415.1; 000460715.1; 001284605.1; 000703865.1; 000829985.1; 000461415.1; 000176615.2; 000482045.1; 000460695.1; 000812785.1; 001277455.1; 000217975.2; 000313405.1; 000305155.2; 000194645.2; 000798395.1; 000408545.1; 000235085.1; 000408445.1; 000703705.1; 000194335.2; 001469815.1; 000492275.1; 000512625.1; 000948905.1; 000617505.2; 000782415.1; 000813165.1; 000782135.1; 000779915.1; 000397265.1; 000021125.1; 000167855.1; 000782395.1; 001269085.1; 000938995.1; 000785775.1; 000358245.2; 001265755.1; 000326185.1; 000485635.1; 000819065.1; 001561675.1; 000714375.1; 001509735.1; 000507645.1; 000776815.1; 000457855.1; 001012535.1; 000408565.1; 000704145.1; 000350985.1; 000782475.1; 000335175.2; 001561495.1; 000713775.1; 000234215.2; 000778465.1; 000459235.1; 001285065.1; 000459975.1; 000807635.1; 000352705.1; 000801185.2; 000752815.1; 000614965.1; 000797795.1; 000615225.2; 000700345.1; 001280405.1; 000249935.2; 001262805.1; 000249875.2; 000462765.2; 000627195.1; 000460215.1; 001284385.1; 000456285.1; 001519975.1; 000619765.2; 001191465.1; 000703325.1; 000522245.1; 000700585.1; 000576655.1; 000713015.1; 001266115.1; 000622465.2; 000485695.1; 000304855.2; 000194685.2; 000267405.2; 000696545.1; 000401755.1; 001284785.1; 001054095.1; 001306715.1; 000159295.1; 000704165.1; 001455385.1; 000713945.1; 000714175.1; 001561335.1; 001309715.1; 000461875.1; 000695155.1; 000446805.2; 000601355.1; 000163215.1; 000352545.1; 001281815.1; 000752935.1; 001285985.1; 000351965.1; 000798475.1; 000326165.1; 000351585.1; 001268945.1; 000777625.1; 000713335.1; 000779975.1; 001432195.1; 000941995.1; 000619425.1; 000418655.1; 000781605.1; 000260475.1; 001420075.1; 000457475.1; 000819085.1; 000752595.1; 000164315.1; 000019425.1; 000936085.2; 000358435.2; 001286225.1; 000522165.1; 000446115.2; 001282035.1; 000357605.2; 001267345.1; 000495035.1; 000350645.1; 001413005.1; 000965575.1; 000223015.2; 000304255.1; 000261385.1; 000273425.1; 000692695.1; 000460015.1; 001286425.1; 001420955.1; 000704705.1; 000776565.1; 000779955.1; 000179795.1; 000408465.1; 001280325.1; 000408105.1; 000714895.1; 000233875.1; 000617985.2; 001563395.1; 000458235.1; 000782575.1; 000351005.1; 000781455.1; 000250195.2; 000782695.1; 001542545.1; 000335155.2; 000781415.1; 001561295.1; 000647475.1; 001014795.1; 000713025.1; 000780755.1; 000965715.1; 000713255.1; 000781875.1; 001283405.1; 000353025.1; 000704365.1; 001561445.1; 001265885.1; 000408685.1; 000194215.2; 000356645.2; 001519755.1; 001268325.1; 001283365.1; 000692555.1; 000304015.2; 001559655.1; 000616225.2; 000471445.1; 000781485.1; 000781555.1; 001283925.1; 001268265.1; 001265295.1; 000752635.1; 000776415.1; 000351545.1; 000353005.1; 001286465.1; 000354555.2; 000615455.2; 000249915.2; 001463205.1; 000616745.1; 000358875.1; 001309645.1; 000776375.1; 000798175.1; 000779275.1; 000249375.2; 000695115.1; 000461995.2; 001284765.1; 001264215.1; 000704785.1; 000768405.1; 000326905.1; 000622285.2; 000614885.2; 000457575.1; 000819355.1; 000617205.1; 000804365.1; 000798095.1; 001309885.1; 000235045.1; 000442065.2; 001269325.1; 000614085.2; 001561635.1; 001056965.1; 000456765.1; 000353145.1; 000713855.1; 000806275.1; 001269025.1; 000461335.1; 000700665.1; 000940255.1; 000782195.1; 000447045.2; 000167915.2; 000704725.1; 000457265.1; 000250315.2; 000798315.1; 000704285.1; 000615085.1; 001420055.1; 001309675.1; 000779175.1; 001412995.1; 000988355.1; 000776855.1; 000249615.2; 000249435.2; 000351605.1; 000459355.1; 000352245.1; 000488835.1; 000703745.1; 000780915.1; 001191115.1; 000456625.1; 000446305.2; 001076455.1; 000352025.1; 001283665.1; 000700305.1; 000616725.2; 001269005.1; 000776505.1; 000965635.1; 000017765.1; 001265165.1; 000225225.2; 000488655.1; 000456865.1; 000692635.1; 000458705.1; 000485715.1; 000522185.1; 000617285.2; 001268985.1; 001519235.1; 000355495.2; 000320155.1; 001561455.1; 001297975.1; 001519475.1; 000166555.2; 000780995.1; 000457595.1; 001266405.1; 000457735.1; 000013265.1; 001283685.1; 000326845.1; 001063835.1; 000798295.1; 000713675.1; 000833635.2; 000188855.2; 000692395.1; 000459755.1; 000488015.1; 000351405.1; 000782595.1; 000193955.2; 000699365.1; 000614725.2; 000408705.1; 000615865.2; 000333215.1; 000316485.1; 000704545.1; 000219515.3; 000704125.1; 000781335.1; 001191195.1; 000713495.1; 001284185.1; 000770275.1; 001183685.1; 000335335.2; 000458475.1; 000262125.1; 000461155.1; 000351425.1; 001309925.1; 001285045.1; 000488715.1; 000704625.1; 000183005.2; 000779265.1; 000770285.1; 000352945.1; 000704525.1; 001266015.1; 000797815.1; 000458255.1; 000776215.1; 001309825.1; 000461255.1; 001561195.1; 000777555.1; 001286565.1; 000616365.1; 000225045.2; 000461295.1; 000704645.1; 000948615.1; 000267085.1; 000303535.2; 001265435.1; 001268385.1; 000708165.1; 000714295.1; 000711485.1; 001563405.1; 000699285.1; 000355395.2; 000007445.1; 000459415.1; 000607285.1; 000488155.1; 001277435.1; 000418635.1; 000458805.1; 000713695.1; 001277715.1; 000364305.1; 000506445.2; 000947315.1; 000951895.1; 001191275.1; 000459115.1; 000408125.1; 001266255.1; 000778705.1; 000782255.1; 000400895.2; 000692595.1; 000690965.1; 001561685.1; 001285965.1; 001419785.1; 000781275.1; 001059795.1; 001284445.1; 000776035.1; 000355475.2; 001285725.1; 000408745.1; 001413415.1; 000713235.1; 000781575.1; 000408205.1; 000701125.2; 001277555.1; 000782535.1; 000351265.1; 000776595.1; 001191425.1; 000358005.2; 000460475.1; 000946755.2; 000704005.1; 000617705.2; 000250095.2; 000249735.2; 000460915.1; 000304835.2; 000779215.1; 000627905.1; 001561755.1; 000267005.2; 000819185.1; 000777065.1; 000408145.1; 000704825.1; 000457205.1; 000352285.1; 001283145.1; 001420125.1; 000714335.1; 001309755.1; 000503335.1; 000351945.1; 000458625.1; 000951755.1; 000351485.1; 000249715.2; 000326745.1; 001285165.1; 000614925.2; 000387805.2; 000503255.1; 000704845.1; 000168095.1; 000249515.2; 000782675.1; 000939955.1; 000354615.2; 001281995.1; 000352425.1; 000503475.1; 000353045.1; 000351285.1; 000164515.1; 000457875.1; 000356125.1; 000711355.1; 000461515.1; 000496345.2; 000529975.1; 000713455.1; 000704425.1; 000226585.1; 000618365.1; 000459275.1; 000316365.2; 000164215.1; 000492255.1; 000408285.1; 000988385.1; 001282155.1; 000457795.1; 001284985.1; 001286365.1; 000305415.2; 001263015.1; 000780115.1; 000618205.2; 000267165.1; 001413605.1; 000460375.1; 000619205.2; 000215265.2; 001282235.1; 001284485.1; 000250255.2; 000354515.2; 000316845.2; 000320215.1; 000496365.2; 000303955.2; 000616425.2; 000831335.1; 000797675.1; 000326365.1; 000948565.1; 000234235.2; 000617225.1; 001183645.1; 000614405.2; 000714955.1; 000460755.1; 000614345.2; 000460055.1; 001012445.1; 000703225.1; 000397725.1; 000187345.2; 000352005.1; 001277575.1; 001285305.1; 000619085.2; 001281915.1; 000459555.1; 000267605.2; 001265255.1; 000488755.1; 000316385.2; 001285205.1; 000692875.1; 000462685.2; 000326565.1; 000627135.1; 000358415.2; 001266225.1; 000179155.1; 000798495.1; 000753115.1; 001284345.1; 000778585.1; 001269285.1; 000627985.1; 001277735.1; 000692435.1; 000779635.1; 000497015.1; 000458375.1; 000456645.1; 000187325.2; 000351045.1; 000782175.1; 000798135.1; 000304035.2; 000351165.1; 000459675.1; 000782555.1; 000008865.1; 000456825.1; 001284825.1; 000358125.1; 000779815.1; 000692835.1; 001421045.1; 000358025.2; 000986635.1; 000511465.1; 000752915.1; 000662395.1; 000948765.1; 000971615.1; 000303775.2; 000460855.1; 000352085.1; 000461815.1; 000714265.1; 001561765.1; 000303515.2; 000249495.2; 000303495.2; 000798455.1; 000488135.1; 000945955.1; 000250435.2; 000459615.1; 000304115.2; 000618245.1; 000458395.1; 000303415.2; 001282065.1; 000704565.1; 000714915.1; 000463605.1; 000148605.1; 001285685.1; 000773435.1; 000267285.2; 001265675.1; 000354475.2; 000599745.2; 000461115.1; 000017985.1; 000355335.2; 001277755.1; 000351085.1; 000163155.1; 000358285.2; 001262895.1; 000488115.1; 000948955.1; 000249295.2; 000285375.1; 000687005.1; 000358045.2; 000352845.1; 000798015.1; 000627885.1; 000326245.1; 001561355.1; 000304055.2; 000777495.1; 001284305.1; 000352745.1; 000308975.2; 000456145.1; 000340275.1; 000461695.1; 001265235.1; 000700365.1; 000777285.1; 000460575.1; 000249275.2; 001052705.1; 000713275.1; 000408025.1; 000700405.1; 000711475.1; 000618305.2; 000267825.2; 000522405.1; 000781315.1; 001309995.1; 000937415.1; 000316905.2; 000506505.1; 001266375.1; 000954045.1; 000618625.1; 000446705.2; 000778095.1; 000462045.2; 000773535.1; 001265315.1; 001463115.1; 000619185.1; 000176695.2; 000456185.1; 000358455.2; 000164255.1; 000617785.1; 001455405.1; 000148365.1; 001268645.1; 000488595.1; 000619065.2; 000458135.1; 000351925.1; 000781515.1; 000410675.2; 000615815.2; 000780375.1; 000355275.2; 001306685.1; 000355175.1; 000461535.1; 000703425.1; 001286045.1; 000752455.1; 000781735.1; 000715035.1; 000462665.2; 000618225.2; 000249195.2; 000618805.1; 000776355.1; 000506825.1; 001284545.1; 000619725.2; 001561815.1; 000619265.2; 000948825.1; 000777975.1; 000776435.1; 001262975.1; 000408665.1; 000779005.1; 000446665.2; 000817375.1; 000354275.2; 001268185.1; 001463455.1; 000818965.1; 001310005.1; 000781045.1; 001267355.1; 000461855.1; 000779515.1; 000358835.2; 001286385.1; 000358315.1; 000711605.1; 000284495.1; 000355595.1; 000214765.3; 000807655.1; 001285605.1; 000617045.2; 000778725.1; 001284105.1; 000222505.2; 001562715.1; 000350685.1; 001265545.1; 000800845.2; 000356945.1; 000776455.1; 001309685.1; 000458825.1; 000773595.1; 000461935.2; 000647815.3; 001058225.1; 000250355.2; 001283285.1; 000268205.1; 000462385.2; 001012585.1; 000446285.2; 000601255.1; 000812645.1; 001284885.1; 000619665.1; 000259695.1; 000316545.2; 000617665.2; 001519555.1; 000797955.1; 000494975.1; 000807575.1; 000617345.1; 000353185.1; 001561705.1; 000781235.1; 000617005.2; 001268445.1; 001309985.1; 000250235.2; 000354875.1; 001285005.1; 000461615.1; 000356065.2; 001266195.1; 000225065.2; 000459095.1; 000316425.2; 001509655.1; 000691085.1; 000833145.1; 001297995.1; 000460995.1; 000457635.1; 000320235.1; 000965655.1; 000462605.2; 000460675.1; 000700505.1; 001191055.1; 001519445.1; 000468515.1; 000353165.1; 001563385.1; 000601175.1; 000618925.2; 000517165.1; 001284665.1; 000352985.1; 000354435.2; 000522105.1; 000800675.1; 000776045.1; 000615655.2; 000350865.1; 000456445.1; 000397645.1; 000780775.1; 000356085.2; 000619625.1; 000250455.2; 000171995.1; 001519685.1; 000457715.1; 000778175.1; 000599825.1; 000778315.1; 001413535.1; 000457975.1; 000355295.1; 000009565.2; 000703365.1; 001191345.1; 000447105.2; 000267945.2; 000780415.1; 000267805.2; 000026545.1; 000352725.1; 001012405.1; 000617465.2; 000461795.1; 000703685.1; 000319975.1; 000782215.1; 000456905.1; 000704805.1; 000446985.2; 000447025.2; 000618465.1; 001283885.1; 000457085.1; 000968515.1; 000781355.1; 000780795.1; 000703305.1; 001285365.1; 000456565.1; 001076105.1; 001441345.1; 000335195.2; 001266075.1; 000316785.2; 001286005.1; 000354755.2; 000800765.1; 000459935.1; 000457495.1; 000242055.1; 000750555.1; 000299255.1; 001191125.1; 000462025.2; 001057065.1; 000798215.1; 000700465.1; 000617185.2; 001440045.1; 000460255.1; 000615415.2; 000704745.1; 000753535.1; 000713265.1; 000462785.2; 000456945.1; 000488535.1; 000190815.1; 000520055.1; 000488635.1; 000250215.2; 000408825.1; 001277595.1; 000167835.1; 000357585.2; 000780075.1; 000407845.1; 000520035.1; 000781035.1; 001284125.1; 000458645.1; 001277675.1; 001039075.1; 000937275.1; 000778915.1; 001519435.1; 001012475.1; 000703985.1; 000632575.1; 000267485.2; 000498275.1; 000305355.1; 000447125.2; 000010765.1; 001057125.1; 000522225.1; 000776775.1; 000326965.1; 000618285.2; 000619105.1; 000407865.1; 000622595.2; 000461355.1; 001561895.1; 000459395.1; 001306675.1; 000947945.1; 000778965.1; 001284425.1; 000485655.1; 000776395.1; 000316805.2; 000614805.2; 000225165.2; 000357785.2; 000194395.2; 000948475.1; 000225145.2; 000462305.2; 000797595.1; 000462345.2; 000780925.1; 001245225.1; 000798335.1; 000714255.1; 000951835.2; 000457755.1; 000172035.1; 000778375.1; 001402805.1; 000952955.1; 000462185.2; 001056025.1; 000456585.1; 000235225.1; 000249175.2; 000457005.1; 000798595.1; 000692375.1; 001265995.1; 000507625.1; 000352145.1; 000779155.1; 000356165.2; 000812765.1; 000459575.1; 000458935.1; 000245515.1; 001441325.1; 000618985.2; 000335015.2; 001283525.1; 000357145.2; 000457895.1; 000704325.1; 000752255.1; 000461495.1; 001562815.1; 001420935.1; 000618585.1; 000966935.1; 001413995.1; 000599785.2; 000215185.2; 000942915.1; 000257275.1; 000355115.2; 000462325.2; 000263915.1; 001268285.1; 000599645.1; 001562345.1; 000319955.1; 000947995.1; 001182885.1; 000614035.2; 000091005.1; 000776635.1; 000819005.1; 000616445.2; 001058205.1; 000249055.2; 001285525.1; 000711595.1; 001455025.1; 001056845.1; 001029125.1; 001265975.1; 000616525.2; 000249475.2; 000614765.2; 000781755.1; 000780535.1; 000780155.1; 000326325.1; 000013305.1; 000456045.1; 000713395.1; 000948515.1; 000781385.1; 000249235.2; 000354375.2; 000817425.1; 000615055.1; 000351225.1; 001309595.1; 001191395.1; 000022345.1; 001012615.1; 001054175.1; 000488675.1; 000456245.1; 000703885.1; 000462145.2; 001286705.1; 000713535.1; 001555495.1; 000460935.1; 000352885.1; 000022665.2; 001413485.1; 000354595.1; 001284525.1; 000725305.1; 000023665.1; 001281885.1; 000618965.1; 000355155.2; 000776675.1; 001012235.1; 000461195.1; 000471145.1; 000457615.1; 000703965.1; 000614465.2; 000782295.1; 001285405.1; 000780675.1; 000408225.1; 001265625.1; 001265735.1; 001283845.1; 001182815.1; 000460075.1; 000357185.2; 000350805.1; 001283825.1; 000619345.1; 000351385.1; 000691045.1; 000614215.2; 000618725.2; 000458685.1; 000471505.1; 000511545.1; 000618685.1; 000357245.1; 000456845.1; 000618265.2; 000350885.1; 000599725.2; 000319935.1; 001283985.1; 000461655.1; 000459855.1; 000460975.1; 000352805.1; 000358535.2; 000940655.1; 000508365.1; 000352165.1; 000615745.2; 001284165.1; 000713105.1; 000692755.1; 000190855.1; 000617025.2; 000778135.1; 000616585.2; 000714025.1; 000488375.1; 001078015.1; 000457165.1; 000147855.3; 000797835.1; 000462125.2; 000618525.1; 001509745.1; 000700085.1; 000781255.1; 001269305.1; 001276585.2; 000352965.1; 000981065.1; 000619485.1; 000326285.1; 000937135.2; 000781215.1; 001281845.1; 000446765.2; 000782755.1; 001012345.1; 000619645.2; 000529355.1; 000357045.2; 000456985.1; 000269645.2; 000242015.1; 000192665.2; 001520375.1; 000461135.1; 000692775.1; 000351845.1; 000704245.1; 000777355.1; 000461575.1; 000974505.1; 000456705.1; 000356225.2; 001281905.1; 000408185.1; 000446685.2; 000488735.1; 000622675.2; 000352685.1; 000700145.1; 000458955.1; 000776995.1; 000303755.2; 000459955.1; 001519735.1; 000618865.1; 000418715.1; 001284265.1; 000488335.1; 000356445.2; 000947935.1; 000965545.1; 000408385.1; 000782655.1; 000326305.1; 000460635.1; 000358595.2; 001286245.1; 000616265.1; 000326445.1; 000459495.1; 000777455.1; 001012315.1; 000190955.1; 000335455.2; 000354955.1; 000267045.1; 001513615.1; 000355315.2; 000355095.2; 000622695.2; 000462585.2; 000617545.2; 001059575.1; 001285145.1; 000303975.2; 000354355.2; 000164355.1; 000415485.1; 000615535.1; 000164275.1; 000397485.1; 000632675.2; 001056815.1; 000616485.2; 000350625.1; 000215145.2; 001244915.1; 000700525.1; 001509675.1; 000713735.1; 000704925.1; 000614685.2; 001262455.1; 000147755.2; 001306575.1; 000819105.1; 000781295.1; 000506585.2; 000163195.1; 000221885.1; 000692715.1; 000798535.1; 000622875.2; 000458215.1; 000005845.2; 000781775.1; 001283545.1; 000249395.2; 001561435.1; 000303795.2; 000459715.1; 000779775.1; 000456725.1; 000700425.1; 000212715.2; 000249215.2; 000777995.1; 000482265.1; 000618565.1; 000619705.2; 001269245.1; 001284945.1; 001077955.1; 000354795.2; 000225025.2; 000614435.2; 000194435.2; 000458455.1; 000414155.2; 000459695.1; 000194535.2; 000461375.1; 000935905.1; 000457325.1; 000354335.2; 000618005.2; 000264215.1; 000781175.1; 001412975.1; 001306585.1; 000026305.1; 001012635.1; 000303475.2; 000456885.1; 000350765.1; 000806175.1; 000351885.1; 000982035.1; 000777195.1; 000250135.2; 001309565.1; 000326385.1; 001563415.1; 000465155.1; 000780475.1; 001007915.1; 000780965.1; 001561605.1; 000457345.1; 001559675.1; 000178755.1; 000456665.1; 000617085.1; 001266275.1; 000316325.2; 000017745.1; 000358955.2; 000817355.1; 001264175.1; 000947975.1; 000297235.3; 000703665.1; 001012255.1; 001039135.1; 000781195.1; 000352605.1; 001291365.1; 000806195.1; 001306655.1; 000946475.1; 000352645.1; 000780275.1; 000460295.1; 000357105.1; 000713525.1; 000615605.2; 000776175.1; 000782515.1; 000936635.1; 000618045.1; 000459535.1; 000618165.2; 000586515.1; 000357505.2; 001283785.1; 000356625.2; 000619405.1; 000943355.2; 000459655.1; 000978815.1; 000781835.1; 001562635.1; 000320195.1; 001285745.1; 000418735.2; 000493595.1; 000407745.1; 001561865.1; 001282295.1; 000316825.2; 001265685.1; 001268885.1; 000619225.2; 000355215.2; 001558995.2; 000299455.1; 000172055.1; 000354915.1; 000819165.1; 000459255.1; 000446905.2; 000619385.1; 000617585.1; 000355075.2; 000351665.1; 001562385.1; 000809145.1; 001265935.1; 000164475.1; 000687105.1; 000267145.1; 000456125.1; 000785865.1; 000948885.1; 000619585.2; 000713045.1; 000167895.2; 000819015.1; 001269185.1; 000408265.1; 000948595.1; 000713615.1; 000753075.1; 001559615.2; 000713095.1; 000597845.1; 000618745.1; 000622635.2; 000617645.2; 000358105.2; 000459995.1; 000936495.1; 000356725.1; 000522385.1; 000446365.2; 000267265.2; 000357965.2; 000615505.1; 000477495.2; 000364365.1; 000320255.1; 000781005.1; 000814145.2; 000831715.1; 000488775.1; 000499485.1; 000460095.1; 001262955.1; 000458765.1; 000711565.1; 001561505.1; 001191525.1; 001559635.1; 000780875.1; 001265765.1; 000358635.2; 000408405.1; 000965565.1; 001052125.1; 000804345.1; 000183345.1; 000191015.1; 000351565.1; 000506465.2; 001012165.1; 000387845.2; 000164575.1; 000456405.1; 000353085.1; 000819345.1; 000356485.2; 000418615.2; 000458555.1; 000784925.1; 000776925.1; 000023365.1; 000190915.1; 000179115.1; 000303915.2; 000988465.1; 001519405.1; 000714055.1; 001413755.1; 001269205.1; 000601195.1; 000408725.1; 000619505.1; 000711515.1; 000462545.2; 001285325.1; 000358775.2; 000778035.1; 000357305.2; 000627235.1; 001077875.1; 000618485.1; 000350185.1; 000285655.3; 000835055.1; 001463645.1; 000261145.1; 000779895.1; 000616325.2; 000458195.1; 000317395.1; 000806265.1; 000457045.1; 000192685.2; 000351325.1; 000699265.1; 000250535.2; 000457245.1; 001052935.1; 000691065.1; 000779715.1; 000776335.1; 001285105.1; 000235105.1; 000320095.1; 000713905.1; 000954035.1; 000233895.1; 001280385.1; 000749545.1; 000194665.2; 000776135.1; 000618665.1; 000461955.2; 000353065.1; 000798275.1; 000780175.1; 000633675.1; 000356985.2; 000358065.2; 000194175.2; 000356665.2; 000781075.1; 001285265.1; 000355455.2; 000319995.1; 000010385.1; 000457105.1; 000326685.1; 000459635.1; 000456505.1; 001268725.1; 001419895.1; 000357365.2; 000498835.2; 000797575.1; 001561215.1; 000354835.2; 000456685.1; 001268245.1; 001285505.1; 001012505.1; 000616605.2; 000522125.1; 000458415.1; 000778785.1; 000235245.1; 000700285.1; 000494955.1; 000446485.2; 001442495.1; 000782775.1; 001562645.1; 001513635.1; 000781095.1; 001281795.1; 000941575.1; 000819225.1; 001520455.1; 000692655.1; 000947985.1; 000352105.1; 000948895.1; 000187365.2; 000458515.1; 000225245.2; 001285845.1; 000457405.1; 000617125.2; 000407945.1; 000027125.1; 000408585.1; 000974405.1; 000249255.2; 000618765.1; 000752155.1; 000459875.1; 000351465.1; 000456085.1; 000267645.2; 000456105.1; 000951955.1; 000782275.1; 000250375.2; 001443175.1; 000171955.1; 000507605.1; 000752755.1; 000498815.2; 000456605.1; 000235065.1; 000190835.1; 000303815.2; 000354775.2; 001268425.1; 000776715.1; 000703765.1; 000781855.1; 001519775.1; 000462845.2; 001447405.1; 001268305.1; 000351365.1; 000446425.2; 000267205.2; 000632635.1; 000249315.2; 000166595.2; 000752895.1; 000617525.2; 001286625.1; 000778395.1; 001191135.1; 000194415.2; 000164595.1; 001309905.1; 000358225.2; 000462065.2; 000615925.2; 001058375.1; 000358145.2; 001012545.1; 001413645.1; 000461215.1; 000351245.1; 000703385.1; 000948605.1; 000692455.1; 000458975.1; 000460515.1; 000303455.2; 001284705.1; 000357525.2; 000397745.1; 000397665.1; 000446385.2; 000267365.2; 000249595.2; 000357645.1; 000622575.2; 000352205.1; 000181755.1; 000564835.2; 000806255.1; 000618105.2; 001182805.1; 000755445.1; 000780455.1; 000752495.1; 000777375.1; 000184185.1; 001285085.1; 000798375.1; 000778955.1; 001043215.1; 001283505.1; 000234275.3; 001561845.1; 000778415.1; 001267335.1; 000446965.2; 001561045.1; 000194295.2; 001561655.1; 000235265.1; 001012175.1; 001309805.1; 000155125.1; 000461595.1; 000458275.1; 000358795.2; 000619285.2; 000267625.2; 001012015.1; 000355015.2; 000618645.1; 000267025.1; 000599805.2; 000267065.1; 000941395.1; 000267705.2; 000249955.2; 000700645.1; 001562655.1; 000249795.2; 000492235.1; 001183665.1; 000713315.1; 000359015.2; 000627795.1; 001268765.1; 000619305.1; 000778685.1; 000146735.1; 000457535.1; 001466755.1; 000164335.1; 000618845.1; 000351145.1; 000457385.1; 000408005.1; 000488815.1; 000320015.1; 000599625.1; 001285645.1; 001284905.1; 000326925.1; 000351065.1; 000357945.2; 000303695.2; 000461015.1; 001521075.1; 000358475.1; 000249415.2; 000460115.1; 001462715.1; 001285025.1; 001308125.1; 001561025.1; 000242035.1; 000488315.1; 000704085.1; 000488695.1; 000408785.1; 000700565.1; 000357265.2; 001283165.1; 001058275.1; 000359055.2; 000408765.1; 001283865.1; 000776155.1; 000352305.1; 000779655.1; 000235125.1; 000622755.2; 001420135.1; 000319855.1; 000752695.1; 000617805.2; 000798115.1; 000965705.1; 000986765.1; 000619605.1; 000462445.2; 001029415.1; 001562855.1; 000460355.1; 000935075.1; 001010195.1; 000446245.1; 000304075.2; 001039215.1; 001562705.1; 000752655.1; 000194255.1; 000258225.1; 000622795.2; 000687065.1; 000319835.1; 000627815.1; 000442085.2; 000513035.1; 000459215.1; 000457915.1; 000461055.1; 000797605.1; 001413565.1; 000615345.1; 001309465.1; 001277615.1; 000352665.1; 001519575.1; 000461755.1; 000522145.1; 000622555.1; 000601215.1; 000460435.1; 000320075.1; 000356825.2; 001562695.1; 000704225.1; 000351785.1; 000798035.1; 000456225.1; 000456745.1; 001521285.1; 000335055.2; 001285245.1; 000319875.1; 000458075.1; 000703805.1; 000354295.1; 000458015.1; 000267505.2; 000352365.1; 000797975.1; 000618345.1; 000779475.1; 001268845.1; 001286485.1; 000458335.1; 001012095.1; 000326605.1; 000351645.1; 000601335.1; 000461075.1; 000340455.2; 000446605.2; 000773575.1; 000006665.1; 000250575.2; 001058935.1; 000460835.1; 000779695.1; 000511525.1; 000355195.2; 000352765.1; 000215225.2; 001309835.1; 000819125.1; 000351725.1; 000622775.2; 000819265.1; 001509665.1; 001432175.2; 000350905.1; 000812325.1; 000619525.1; 001420045.1; 000782735.1; 001283425.1; 001440615.1; 001440525.1; 000713185.1; 000252805.2; 000456465.1; 000798555.1; 000781675.1; 000268125.1; 001268225.1; 000304815.2; 001413825.1; 000778105.1; 001059735.1; 000354415.2; 000471165.1; 000333175.1; 001263065.1; 000320045.1; 000353545.1; 000158395.1; 000776835.1; 000713975.1; 000456005.1; 001561735.1; 001277655.1; 000488395.1; 000798435.1; 000302735.1; 000752135.1; 000025745.1; 001285285.1; 000235165.1; 000357865.2; 000299475.1; 000736735.1; 000753155.1; 001285225.1; 000352485.1; 000965555.1; 000446175.2; 000498235.1; 000734955.1; 000461235.1; 000778565.1; 001059655.1; 000798255.1; 000704345.1; 000267245.2; 001012265.1; 000352445.1; 000714995.1; 000326425.1; 000692495.1; 001519125.1; 000703825.1; 000351105.1; 001286605.1; 000188835.2; 000616385.2; 000358515.2; 000188875.2; 001262905.1; 000779295.1; 000778485.1; 000618185.1; 000176575.2; 000456345.1; 000351205.1; 001063395.1; 000303875.2; 000326265.1; 000303895.2; 000703525.1; 000730345.1; 000704765.1; 000250415.2; 000458095.1; 000691105.1; 000356185.2; 000307205.1; 000633655.1; 000695175.1; 001286025.1; 000779875.1; 000731455.1; 000462865.2; 000457435.1; 000319915.1; 000460895.1; 000267465.2; 000690985.1; 000713505.1; 000948445.1; 000700205.1; 000235285.1; 001265495.1; 000752315.1; 000806245.1; 000632595.2; 000460275.1; 000303315.2; 000460415.1; 000407825.1; 000522345.1; 000782095.1; 000835045.1; 000326345.1; 001519365.1; 000320115.1; 000780595.1; 000777655.1; 000357325.1; 000446645.2; 001056875.1; 000731355.1; 000456965.1; 000948835.1; 000615575.2; 001012025.1; 000458605.1; 000215285.2; 000797935.1; 000358695.2; 000459375.1; 000780815.1; 000305435.2; 000461835.1; 001265925.1; 000703205.1; 000974885.1; 000258025.1; 000249555.2; 000458435.1; 000305375.2; 001284205.1; 000303655.2; 000488295.1; 000320035.1; 000457305.1; 000407705.1; 001191355.1; 000617405.2; 000599705.1; 000408485.1; 001191505.1; 000940115.1; 000350945.1; 000714185.1; 001519525.1; 000713175.1; 000355575.2; 001268205.1; 000622485.2; 001269125.1; 000495055.1; 000618505.1; 000250275.2; 001309535.1; 000175755.1; 000700545.1; 001266175.1; 000690945.1; 001286305.1; 000397685.1; 000358655.2; 000249115.2; 001309775.1; 000627095.1; 000812385.1; 000461175.1; 000249075.2; 001561115.1; 000618905.2; 000458575.1; 001562335.1; 000460535.1; 000241975.1; 000163175.1; 000014845.1; 000618425.1; 001277395.1; 001561415.1; 000456205.1; 000350045.2; 000458295.1; 000460655.1; 000768505.1; 001277775.1; 001283605.1; 001277635.1; 000506485.2; 001463535.1; 000164455.1; 000351685.1; 000462805.2; 000797875.1; 001285625.1; 000303275.2; 001413355.1; 000779075.1; 000407685.1; 000619465.2; 000303295.2; 000711525.1; 001265175.1; 000622535.2; 000616005.2; 000358165.2; 000780695.1; 000522365.1; 000357285.2; 001012575.1; 001519135.1; 001561175.1; 001306635.1; 000303235.2; 000622505.2; 000352625.1; 000358185.2; 000418755.2; 001282025.1; 001266305.1; 000320275.1; 000817345.1; 001012395.1; 001051135.1; 000812345.1; 000798415.1; 000498875.2; 000350785.1; 000187285.4; 000220005.2; 001563715.1; 000354995.2; 001268465.1; 000627075.1; 000316745.2; 000357465.2; 000459915.1; 000777025.1; 000459475.1; 001284745.1; 000357165.1; 000713585.1; 001077865.1; 001058175.1; 001283945.1; 001517685.1; 001191105.1; 000777155.1; 001012005.1; 000692675.1; 000462225.2; 001285485.1; 000777165.1; 000696835.1; 000765435.1; 000614565.2; 000780495.1; 000615785.2; 000780095.1; 000619025.1; 001282195.1; 000446885.2; 001561205.1; 000768425.1; 000357625.2; 001053065.1; 000780845.1; 000498795.2; 000459055.1; 001191045.1; 000352225.1; 001187545.1; 001285185.1; 001268745.1; 001463585.1; 000460815.1; 000258635.1; 000249855.2; 001285345.1; 000352325.1; 000461455.1; 001521455.1; 000460775.1; 001005605.1; 000785255.1; 000780735.1; 000457455.1; 000713575.1; 001309895.1; 001562765.1; 000488415.1; 001268545.1; 000459775.1; 000458845.1; 000320135.1; 000601235.1; 001283745.1; 000700165.1; 000456545.1; 001519355.1; 001519215.1; 000351025.1; 000782315.1; 000249895.2; 001063685.1; 000948555.1; 000711415.1; 001521575.1; 000478705.1; 000622855.2; 001309965.1; 000335115.1; 000782495.1; 000326585.1; 001561035.1; 000227625.1; 000777895.1; 001269225.1; 000259385.1; 001263735.1; 001268685.1; 001561595.1; 000326405.1; 000356785.2; 000026285.1; 000948875.1; 000778355.1; 000354455.1; 001419865.1; 001286685.1; 000692415.1; 000462085.2; 000474845.1; 000418695.2; 000250115.2; 001281925.1; 000326825.1; 000187385.2; 000019385.1; 000326465.1; 000627215.1; 000488795.1; 000352385.1; 000618885.1; 000359095.2; 000358265.2; 000753195.1; 000249335.2; 000781965.1; 000353105.1; 000408345.1; 000249695.2; 001307215.1; 000492655.1; 000632555.1; 000305175.1; 000732965.1; 000752235.1; 000249675.2; 000616685.2; 000407885.1; 000460955.1; 000316765.2; 000700705.1; 000461915.2; 000797585.1; 000357485.2; 000818985.1; 001283805.1; 000457655.1; 000485675.1; 000627255.1; 000010745.1; 000938695.1; 000511485.1; 000356605.2; 000615145.1; 000332755.1; 000446095.2; 000303595.2; 000234315.2; 000408425.1; 000459295.1; 000462245.2; 000408645.1; 000249575.2; 001440495.1; 000506845.1; 000457025.1; 000164435.1; 000456365.1; 001057225.1; 001466815.1; 000935515.1; 000351805.1; 000171935.1; 000355355.1; 000488615.1; 000797775.1; 000713115.1; 001265875.1; 000616345.2; 001280345.1; 000352045.1; 000179075.1; 000408085.1; 000614645.2; 000250075.2; 000619045.2; 000713655.1; 000671295.1; 001191025.1; 000948625.1; 000358205.2; 000622715.2; 000779835.1; 000457065.1; 001413555.1; 000615965.2; 000622615.2; 001283725.1; 000408165.1; 001283265.1; 000458745.1; 000351985.1; 001285705.1; 000335355.2; 000326785.1; 000166575.2; 000632655.1; 000350665.1; 000617365.2; 000782235.1; 000752775.1; 000753275.1; 000762385.1; 000456305.1; 000408505.1; 000446625.2; 000797995.1; 000357925.2; 000460455.1; 000190895.1; 000713825.1; 001284565.1; 000461775.1; 000187305.2; 001021005.2; 000458785.1; 000335095.2; 001269105.1; 000797755.1; 000780315.1; 000777605.1; 000778475.1; 000459835.1; 001266125.1; 000446225.2; 000410655.2; 000692515.1; 000617325.1; 001308065.1; 001266095.1; 001520115.1; 000316465.1; 001521415.1; 000703285.1; 001555635.1; 000782355.1; 000812235.1; 000456805.1; 001265415.1; 000267345.2; 001286325.1; 000776115.1; 001283485.1; 000615305.1; 000387785.2; 000785325.1; 001561095.1; 000276745.1; 000355035.2; 000797895.1; 000456325.1; 000305455.2; 000627835.1; 001268605.1; 000778435.1; 000700445.1; 001413425.1; 000446505.2; 000616645.2; 000776535.1; 000615265.1; 000807555.1; 000704385.1; 001012355.1; 001059485.1; 001286545.1; 000618445.1; 000250475.2; 000776055.1; 000179175.1; 000249975.2; 000303335.2; 000459735.1; 000781115.1; 001262795.1; 000326765.1; 000356845.2; 001286585.1; 000351745.1; 000936245.1; 000359035.2; 000457675.1; 000352825.1; 000446075.2; 001516935.2; 000356105.2; 000446195.2; 000462725.2; 001515725.1; 000713785.1; 000700325.1; 000457995.1; 000752295.1; 000353125.1; 000770055.1; 001284245.1; 000619165.1; 000777415.1; 001561365.1; 000303355.2; 000781955.1; 000779055.1; 000398885.1; 000692535.1; 001005685.1; 000408065.1; 000616665.2; 000965625.1; 000974575.1; 000776195.1; 001268805.1; 000456785.1; 000460315.1; 000019645.1; 001277495.1; 000352465.1; 000326505.1; 000351525.1; 000459035.1; 000267905.2; 000616625.1; 000782635.1; 000357125.1; 000485735.1; 000462465.2; 000457835.1; 000488355.1; 000618705.2; 000779365.1; 000267225.2; 000948005.1; 000522285.1; 001191185.1; 000617065.1; 001191455.1; 000704685.1; 000190795.1; 000258785.1; 000163235.1; 000354535.2; 000351125.1; 001463195.1; 001284225.1; 000713895.1; 001286645.1; 000779125.1; 000974825.1; 000351505.1; 000618385.1; 000781145.1; 000779425.1; 000355055.1; 000357005.2; 000752175.1; 001269165.1; 000462265.2; 000462705.2; 001268505.1; 000458535.1; 001039415.1; 000700605.1; 000616505.2; 000708145.1; 001285665.1; 000461975.2; 000781915.1; 000459895.1; 000700725.1; 000357905.2; 000616035.2; 000270105.1; 000780615.1; 000782335.1; 001446455.1; 000267885.2; 000752435.1; 000267665.2; 000478215.1; 000267125.1; 001281855.1; 000965665.1; 000250155.2; 000782615.1; 001262785.1; 001308165.1; 000010485.1; 000457145.1; 000462825.2; 000460795.1; 000488035.1; 000801165.1; 000798235.1; 000618605.1; 000488495.1; 000700245.1; 000780255.1; 000355255.2; 000461555.1; 000619685.2; 000326625.1; 000939255.1; 001442595.1; 000456425.1; 000335435.2; 000176675.2; 000460235.1; 000267785.2; 000259425.1; 000458895.1; 000488515.1; 000931565.1; 000779585.1; 000462625.2; 000801205.1; 000397625.1; 000777945.1; 000779935.1; 000194355.2; 001562835.1; 000627155.1; 001284625.1; 000752195.1; 000713195.1; 000166535.2; 000178795.1; and 000355615.2.

To select reference genomes to use for probe design, all 295 RefSeq genomes were included, as they were all high quality, complete, and fairly diverse. Also, 1,418 Genbank genomes were selected for inclusion in the following way. All 1,124 singleton (i.e., not clustered with other genomes) Genbank genomes were retained. Then, the largest Genbank genome from each of the 361 multi-genome clusters were included, if and only if there was no RefSeq-equivalent genome in the cluster. This added an additional 294 Genbank genomes. This final set of 1,713 genomes represented a large, diverse collection, with references from all eight major clades of E. coli (Table 2), as determined by the tool ClermonTyping (Beghain, Johann, Antoine Bridier-Nahmias, Hervé Le Nagard, Erick Denamur, and Olivier Clermont. 2018.“ClermonTyping: An Easy-to-Use and Accurate in Silico Method for Escherichia Genus Strain Phylotyping.” Microbial Genomics 4 (7). doi.org/10.1099/mgen.0.000192), and 515 distinct multi-locus sequence types (data not shown), as determined by the tool mlst (see PubMLST website developed by K. Jolley (Jolley & Maiden 2010, BMC Bioinformatics, 11:595)).

The RefSeq accession numbers for the 295 RefSeq genomes (“the 295 RefSeq genomes”) were the following, where the identifier “GCF” (i.e., the designation as RefSeq assemblies) that precedes each number has been omitted: 000005845.2; 000006925.2; 000007405.1; 000007445.1; 000008865.1; 000009565.1; 000010245.2; 000010385.1; 000010485.1; 000010745.1; 000010765.1; 000012005.1; 000012025.1; 000013265.1; 000013305.1; 000013585.1; 000014845.1; 000017745.1; 000017765.1; 000017985.1; 000019385.1; 000019425.1; 000019645.1; 000020185.1; 000021125.1; 000022225.1; 000022245.1; 000022345.1; 000022665.1; 000023365.1; 000023665.1; 000025165.1; 000025745.1; 000026245.1; 000026265.1; 000026325.1; 000026345.1; 000026545.1; 000027125.1; 000091005.1; 000092525.1; 000147855.2; 000148365.1; 000148605.1; 000183345.1; 000184185.1; 000210475.1; 000212715.2; 000219515.2; 000227625.1; 000233875.1; 000233895.1; 000245515.1; 000257275.1; 000258025.1; 000258145.1; 000262125.1; 000270105.1; 000283715.1; 000284495.1; 000285655.3; 000299255.1; 000299455.1; 000299475.1; 000332755.1; 000350185.1; 000468515.1; 000493755.1; 000499485.1; 000520035.1; 000520055.1; 000597845.1; 000599625.1; 000599645.1; 000599665.1; 000599685.1; 000599705.1; 000662395.1; 000671295.1; 000714595.1; 000725305.1; 000730345.1; 000732965.1; 000743255.1; 000743955.1; 000743995.1; 000750555.1; 000784925.1; 000800215.1; 000800765.1; 000800845.1; 000801165.1; 000801185.2; 000801205.1; 000803705.1; 000813165.1; 000814145.2; 000819645.1; 000827105.1; 000829985.1; 000830035.1; 000831565.1; 000833145.1; 000931565.1; 000952955.1; 000953035.1; 000953515.1; 000967155.2; 000968515.1; 000971615.1; 000974405.1; 000974465.1; 000974505.1; 000974535.1; 000974575.1; 000974825.1; 000974865.1; 000974885.1; 000981485.1; 000986765.1; 000987875.1; 000988355.1; 000988385.1; 000988465.1; 001007915.1; 001020945.2; 001021005.2; 001021855.1; 001027225.1; 001029125.1; 001039415.1; 001043215.1; 001051135.1; 001183645.1; 001183665.1; 001183685.1; 001276585.2; 001280325.1; 001280345.1; 001280385.1; 001280405.1; 001307215.1; 001308065.1; 001308125.1; 001308165.1; 001420935.1; 001420955.1; 001442495.1; 001455385.1; 001469815.1; 001485455.1; 001513615.1; 001513635.1; 001515725.1; 001518855.1; 001542675.2; 001544635.1; 001558295.1; 001558995.2; 001559615.2; 001559635.1; 001559655.1; 001559675.1; 001566335.1; 001566615.1; 001566635.1; 001566655.1; 001566675.1; 001577325.1; 001578125.1; 001579965.1; 001580175.1; 001593565.1; 001596115.1; 001610755.1; 001612475.1; 001612495.1; 001617565.1; 001618325.1; 001618345.2; 001618365.1; 001644725.1; 001644745.1; 001650275.1; 001650295.1; 001651925.1; 001651945.1; 001651965.1; 001660565.1; 001660585.1; 001663075.1; 001663475.1; 001675145.1; 001677475.1; 001677495.1; 001677515.1; 001678925.1; 001678965.1; 001679985.1; 001682305.1; 001683435.1; 001693315.1; 001693635.1; 001695515.1; 001721125.1; 001721205.1; 001721225.1; 001721525.1; 001723505.1; 001735705.1; 001750845.1; 001753445.1; 001753465.1; 001753485.1; 001753505.1; 001753525.1; 001753545.1; 001753565.1; 001806265.1; 001806285.1; 001860505.1; 001865295.1; 001886535.1; 001886555.1; 001886575.1; 001886755.1; 001886935.1; 001888075.1; 001890205.1; 001890225.1; 001890245.1; 001890265.1; 001890285.1; 001890305.1; 001890325.1; 001890345.1; 001890365.1; 001900295.1; 001900315.1; 001900335.1; 001900355.1; 001900375.1; 001900395.1; 001900415.1; 001900435.1; 001900455.1; 001900475.1; 001900495.1; 001900515.1; 001900535.1; 001900555.1; 001900575.1; 001900595.1; 001900615.1; 001900635.1; 001900655.1; 001900675.1; 001900695.1; 001900715.1; 001900735.1; 001900775.1; 001900795.1; 001900815.1; 001900835.1; 001900885.1; 001900905.1; 001900925.1; 001900945.1; 001900965.1; 001900985.1; 001901005.1; 001901025.1; 001901045.1; 001901065.1; 001901085.1; 001901105.1; 001901125.1; 001901145.1; 001901165.1; 001901185.1; 001901215.1; 001901315.1; 001901365.1; 001901405.1; 001901425.1; 001901445.1; 001901465.1; 001932515.1; 001936315.1; 001938625.1; 001969285.1; 001999185.1; 900092615.1; 900096795.1; and 900096845.1.

The 1,713 genomes were then uniformly re-annotated with the Broad Institute prokaryotic genome pipeline (Valentino, Michael D., Abigail Manson McGuire, Jason W. Rosch, Paulo J. M. Bispo, Corinna Burnham, Christine M. Sanfilippo, Robert A. Carter, et al. 2014. “Unencapsulated Streptococcus pneumoniae from Conjunctivitis Encode Variant Traits and Belong to a Distinct Phylogenetic Cluster.” Nature Communications 5 (November): 5411; Schreiber, Henry L., 4th, Matt S. Conover, Wen-Chi Chou, Michael E. Hibbing, Abigail L. Manson, Karen W. Dodson, Thomas J. Hannan, et al. 2017. “Bacterial Virulence Phenotypes of Escherichia Coli and Host Susceptibility Determine Risk for Urinary Tract Infections.” Science Translational Medicine 9 (382). doi.org/10.1126/scitranslmed.aaf1283). Genes were clustered into orthogroups using SynerClust (Georgescu, Christophe H., Abigail L. Manson, Alexander D. Griggs, Christopher A. Desjardins, Alejandro Pironti, Ilan Wapinski, Thomas Abeel, Brian J. Haas, and Ashlee M. Earl. 2018. “SynerClust: A Highly Scalable, Synteny-Aware Orthologue Clustering Tool.” Microbial Genomics. doi.org/10.1099/mgen.0.000231), which resulted in a total of 174,584 orthogroups containing 8,334,026 total genes. As the computational time to analyze all these orthogroups using CATCH was prohibitive, orthogroups found in fewer than three genomes were filtered out, leaving 64,146 orthogroups (containing a total of 8,165,358 genes). In order to ensure that the set contained all potential instances of key genes important in clinically relevant E. coli, all instances of orthogroups containing instances of 59 Pfam domains of interest, obtained from a curated list (Table 5), were retained. Using this list, a total of 2,434 orthogroups (3,479 genes) were added back that were found in fewer than three genomes. The 5 final set contained 64,580 orthogroups comprising 8,168,837 genes.

TABLE 5
Pfam domains of interest

| | Pfam Accession |
|---|---|
| Description | No. |
| Usher | PF00577 |
| Chaperone | PF00345 |
| Chaperone | PF02753 |
| Subunit | PF04449 |
| Subunit | PF06551 |
| Subunit | PF00419 |
| Subunit | PF09255 |
| Subunit | PF02432 |
| Subunit | PF05229 |
| Adhesin | PF07434 |
| Adhesin | PF03627 |
| Adhesin | PF05229 |
| Adhesin | PF04619 |
| WaaL | PF04932 |
| WaaJ | PF01501 |
| WaaJ | PF08437 |
| WaaI | PF01501 |
| WaaI | PF0843 |
| WaaA | PF00534 |
| WaaA | PF04413 |
| YibD | PF00535 |
| rfbA | PF00483 |
| WaaY | PF06176 |
| FimE | PF00589 |
| FimB | PF00589 |
| HbiF | PF00589 |
| Fis | PF02954 |
| HU | PF00216 |
| H-NS | PF00816 |
| StpA | PF00816 |
| IHF | PF00216 |
| Lrp | PF01037 |
| ibeA | PF12831 |
| ibeT | PF03553 |
| OmpR | PF00072 |
| OmpR | PF00486 |
| OmpX | PF13505 |
| RpoS | PF00140 |
| RpoS | PF04542 |
| RpoS | PF04539 |
| RpoS | PF04545 |
| CRP | PF00027 |
| CRP | PF00325 |
| SlyA | PF01047 |
| NanR | PF07729 |
| NanR | PF00392 |
| NagC | PF01047 |
| NagC | PF00480 |
| LrhA | PF00126 |
| LrhA | PF03466 |
| RelA | PF13291 |
| RelA | PF13328 |
| RelA | PF04607 |
| RelA | PF02824 |
| DskA | PF01258 |
| PilQ | PF00263 |
| PilQ | PF03958 |
| SfaB | PF03333 |
| SfaX | PF01047 |

In order to reduce design constraints in CATCH, thereby decreasing computational cost and time, orthogroups were clustered using UCLUST (Edgar, Robert C. 2010. “Search and Clustering Orders of Magnitude Faster than BLAST.” Bioinformatics 26 (19): 2460-61.), with an 80% nucleotide sequence identity threshold. This generated one or more clusters of genes within each orthogroup, in which all cluster members had ≥80% nucleotide sequence identity to one other. This generated 87,218 gene clusters from the 64,580 orthogroups. These gene clusters were the input for CATCH probe design.

CATCH (Metsky, Hayden C., Viral Hemorrhagic Fever Consortium, Katherine J. Siddle, Adrianne Gladden-Young, James Qu, David K. Yang, Patrick Brehio, et al. 2019. “Capturing Sequence Diversity in Metagenomes with Comprehensive and Scalable Probe Design.” Nature Biotechnology. doi.org/10.1038/s41587-018-0006-x) was run with the following parameters: 2 bp mismatch allowed; 25 bp cover extension; expand “N” to ACGT; 30 bp island of exact match; 60-75 bp length. In addition, three Enterobacteriaceae genomes were used to “blacklist” probes that matched (i.e., ≤ 8 total mismatches) off-target sequences: Citrobacter freundii CFNIH1, Salmonella enterica subsp. enterica serovar Typhi str. CT18, and Klebsiella pneumoniae subsp. pneumoniae HS11286. These genomes were downloaded from NCBI Refseq. CATCH generated 911,618 unique probe sequences (a number of duplicate probes were generated because CATCH was run to generate probes per gene cluster, and all but one copy of each set of duplicate probes were removed from the probe set).

In addition to using the three genomes to “blacklist” probes, an additional set of filters was applied to remove probes that might still capture off-target sequences. The blastn tool from BLAST+ was used to search for identity against the NCBI prokaryote reference genome database (downloaded in October 2017). Probe sequences were used as queries with the following parameters: max_target_seqs 30; evalue 1e-5; qcov_hsp_perc 80; perc_identity 80. Using these results, probes were removed that had matches of 65 bp or more to: 1)≥100 references in the database (1,798 probes removed); 2) Bacteroidetes references (2,470 probes removed); or 3) Firmicutes references (14,935 probes removed). After this filtering, a total of 892,415 probes (provided in the Sequence Listing as SEQ ID NOs: 1 to 892415) remained that were unlikely to hit other commonly found and abundant bacterial species in the human gut.

### In Silico Probe Set Validation

In order to verify that the probe set captured the vast majority of genes in the E. coli pangenome, blastn from BLAST+ was used to query the probe sequences against the entire pan-genome from the set of 1,713 references, which included genes that had previously been filtered out at the probe design stage. The probe sequences were used as queries for blastn with the following parameters: max_target_seqs 30; evalue le-5; qcov_hsp_perc 80; perc_identity 80. Alignments with>65 bp length and no more than 8 mis-matches were retained in the entire alignment. The probe set was considered to capture a gene if one or more probes met these criteria for the gene.

### Probe synthesis

Probes were synthesized by Roche, though the probe set was not specifically tailored to their technology and could be synthesized by other manufacturers. All probes could be synthesized, although 330,387 (37%) probes had one or more bases truncated from the 3′ end. The average number of bases trimmed per probe was 1.27±2.16. Only 5,423 probes had 10 or more bases trimmed. All of the most highly truncated probes had low nucleotide complexity, primarily due to long stretches of homopolymers. The average length of synthesized probes was 73.7±2.16 bp. As these changes were unlikely to affect the performance of the probe set as a whole, this slightly modified probe set was used in the experiments.

### Creation of Four Strain E. coli Mock Community

Four phylogenetically distinct E. coli strains, namely H10407 (clade A), E24337A (clade B1), UTI89 (clade B2), and Sakai (clade E), were cultured separately overnight at 37° C. in 2 mL of liquid LB media with shaking at 200 rpm. The bacterial number in each culture was estimated via optical density and then combined at a ratio of 80% H10407, 15% UTI89, 4.9% Sakai, and 0.1% E24337A. Genomic DNA was then extracted from this mock community using the Qiagen MagAttract DNA Isolation Kit (Hilden, Germany), following manufacturer's protocols. In two separate tubes, human genomic DNA was then added to the extracted E. coli DNA for final ratios of 99% human/1% E. coli (weight/weight).

### Library Construction and Sequencing for Mock Community

The Nextera XT library construction kit (Illumina) was used to generate sequencing libraries following the manufacturer's recommended protocol. To enrich E. coli sequences in the mock library (˜100 ng into the HC reaction), hybrid capture (HC) was performed using a Roche SeqCap EZ Hypercap kit with the designed custom capture probe set. Hybridization and target capture followed the SeqCap kit instructions with the modifications that the probe pool was diluted 1:2 to 500 pmol/ul before use and custom Nextera adapter blocking oligonucleotides (Metsky, Hayden C., Viral Hemorrhagic Fever Consortium, Katherine J. Siddle, Adrianne Gladden-Young, James Qu, David K. Yang, Patrick Brehio, et al. 2019. “Capturing Sequence Diversity in Metagenomes with Comprehensive and Scalable Probe Design.” Nature Biotechnology. doi.org/10.1038/s41587-018-0006-x) were substituted for the SeqCap HE Universal adapter and index blocking oligonucleotides. After hybridization (18 h), bead capture and washes, 15 cycles of polymerase chain reaction (PCR) were performed with generic universal Illumina P7 and P5 primers. The final libraries were quantified by Qubit fluorometry (Thermo Fisher Scientific) and the size distribution analyzed by TapeStation electrophoresis (Agilent) prior to Illumina sequencing. Then, pre- and post-HC libraries were run on an Illumina HiSeqX, generating 21,460,598 and 75,576,717 paired-end 151 bp reads for pre- and post-HC libraries, respectively.

### Analysis of Four-Strain E. coli Mock Community

In order to compare analyses on samples with the same sequencing depth, post-hybrid capture (HC) data was first downsampled to the same sequencing depth (approximately 20,000,000 paired-end reads) as pre-HC data using Picard-Tools (broadinstitute.github.io/picard/). Data were analyzed with FastQC (Andrews, Simon, and Others. 2010. “FastQC: A Quality Control Tool for High Throughput Sequence Data.” Babraham Bioinformatics, Babraham Institute, Cambridge, United Kingdom) and MultiQC (Ewels, Philip, Måns Magnusson, Sverker Lundin, and Max Käller. 2016. “MultiQC: Summarize Analysis Results for Multiple Tools and Samples in a Single Report.” Bioinformatics 32 (19): 3047-48) for quality metrics. As Picard-Tools indicated that post-HC libraries had PCR duplication, both pre- and post-HC data were de novo deduplicated with FastUniq (Xu, Haibin, Xiang Luo, Jun Qian, Xiaohui Pang, Jingyuan Song, Guangrui Qian, Jinhui Chen, and Shilin Chen. 2012. “FastUniq: A Fast de Novo Duplicates Removal Tool for Paired Short Reads.” PloS One 7 (12): e52249).

Following downsampling and deduplication, relative abundances and depth of coverage for each of the four strains were estimated using the tool StrainGST from the tool suite StrainGE (Dijk, Lucas R. van, Bruce J. Walker, Timothy J. Straub, Colin J. Worby, Alexandra Grote, Henry L. Schreiber, Christine Anyansi, et al. 2021. “StrainGE: A Toolkit to Track and Characterize Low-Abundance Strains in Complex Microbial Communities.” Cold Spring Harbor Laboratory. doi.org/10.1101/2021.02.14.431013). First, a StrainGST database was built containing just the reference genomes of the four strains of E. coli in the mock mixture (i.e., H10407, UTI89, Sakai, and E24377A) all downloaded from NCBI Genbank. Then, both pre- and post-HC data were k-merized and StrainGST was run (without k-mer fingerprinting) against the database to determine the relative abundances and depth of coverages for all four strains.

Coverage levels for each of the four strains were obtained by aligning downsampled and deduplicated data with Bowtie2 (Langmead, Ben, and Steven L. Salzberg. 2012. “Fast Gapped-Read Alignment with Bowtie 2.” Nature Methods 9 (4): 357-59.) with default parameters to a concatenation of all four strains' reference genomes. Only properly paired aligned reads with a minimum mapping quality (MQ) of 5 were retained with samtools (htslib.org/). This filtering was done to exclude reads and regions of the genomes where reads aligned equally well to different strains, with the goal of reducing bias in less abundant strains due to sequence identity to sequences deriving from the more abundant strains. Then, coverages of MQ>5 reads were assessed using Bedtools (bedtools.readthedocs.io/en/latest/).

To determine probe hybridization sites on each strain's reference genome, all probe sequences were aligned using Bowtie2 to each of the four reference genomes, individually. The intervals where probes aligned were designated as putative probe hybridization sites. To determine coverage levels in relation to probe hybridization sites, pre- and post-HC calculated coverages were compared with these putative probe hybridization sites using Bedtools and a custom R script.

Downsampled and deduplicated data were used to generate metagenomic assemblies. First, pre- and post-HC data were digitally normalized with the program khmer (Crusoe, Michael R., Hussien F. Alameldin, Sherine Awad, Elmar Boucher, Adam Caldwell, Reed Cartwright, Amanda Charbonneau, et al. 2015. “The Khmer Software Package: Enabling Efficient Nucleotide Sequence Analysis.” F1000Research 4 (September): 900.). Then, downsampled data were processed with Trim Galore (bioinformatics.babraham.ac.uk/projects/trim_galore/) to remove leftover adapter content. Then, as the background was 99% human DNA, the normalized data were cleaned of human data using Bowtie2 (with “very sensitive” pre-set was used; i.e., -D 20 -R 3 -N 0 -L 20 -i S, 1, 0.50; where -D <int> means give up extending after <int> failed extends in a row, -R <int> means for reads with repetitive seeds, try <int> sets of seeds, -N <int> means max # mismatches in seed alignment can be 0 or 1, -L <int> means length of seed substrings; must be >3, <32, and i <func> means interval between seed substrings w/r/t read len) against the hg38 reference. Reads that did not align to the human genome were then assembled with MetaSPAdes (Nurk, Sergey, Dmitry Meleshko, Anton Korobeynikov, and Pavel A. Pevzner. 2017. “metaSPAdes: A New Versatile Metagenomic Assembler.” Genome Research 27 (5): 824-34.) with default parameters. Contigs and scaffolds smaller than 1 kb were removed. Then, GAEMR (software.broadinstitute.org/software/gaemr/) was used to assess assembly metrics and determine the taxonomy of each contig/scaffold.

### Nucleic Acid Extraction, Library Construction, and Sequencing for Clinical Stool Samples

Stool was collected at home and stored in ethanol until nucleic acid extraction. Nucleic acids were extracted using the Chemagic Blood Kit (CMG-1091), along with separately purchased lysis reagents for the upfront prep (lysozyme, Pro K, and RLT Buffer from Qiagen). The resulting ˜100 μL of total nucleic acids was split in half, cleaning up ˜50 μL for DNA and ˜50 μL for RNA. For the cleanup of the DNA aliquot, SUPERase was used followed by SPRI clean-up with Agencourt AMPure beads. For the cleanup of the RNA aliquot, DNase was used, followed by a SPRI clean-up with Agencourt AMPure beads. DNA-Seq libraries were prepared from 100-120 ng genomic DNA samples using bead-linked transposomes for tagmentation (Illumina DNA Prep or DNA Prep with Enrichment kits) following the manufacturer's instructions, with the modification generic custom Nextera primer pairs with 8-base unique dual indexes were used for PCR amplification (9 cycles).

To enrich E. coli sequences in DNA and RNA libraries, multiplex solution hybrid capture (HC) was performed using a Roche SeqCap EZ Hypercap kit with the hybrid capture probe set. DNA-Seq libraries were processed in pools of 8 libraries (˜200 ng each). RNA-Seq libraries were prepared as pools of multiplex RNAtag-Seq libraries from 12 RNA samples (Shishkin, Alexander A., Georgia Giannoukos, Alper Kucukural, Dawn Ciulla, Michele Busby, Christine Surka, Jenny Chen, et al. 2015. “Simultaneous Generation of Many RNA-Seq Libraries in a Single Reaction.” Nature Methods 12 (4): 323-25; Bhattacharyya, Roby P., Nirmalya Bandyopadhyay, Peijun Ma, Sophie S. Son, Jamin Liu, Lorrie L. He, Lidan Wu, et al. 2019. “Simultaneous Detection of Genotype and Phenotype Enables Rapid and Accurate Antibiotic Susceptibility Determination.” Nature Medicine 25 (12): 1858-64) and amplified by 14 cycles of PCR to generate at least 100 (mean 140) ng of each library pool for HC (one 24-plex pool per reaction). Hybridization and target capture was completed according to the SeqCap kit manufacturer instructions except that the probe pool was diluted 1:2 to 500 pmol/ul before use and custom Nextera adapter blocking oligonucleotides (Metsky, Hayden C., Viral Hemorrhagic Fever Consortium, Katherine J. Siddle, Adrianne Gladden-Young, James Qu, David K. Yang, Patrick Brehio, et al. 2019. “Capturing Sequence Diversity in Metagenomes with Comprehensive and Scalable Probe Design.” Nature Biotechnology.doi.org/10.1038/s41587-018-0006-x) were substituted for the SeqCap HE Universal adapter and index blocking oligonucleotides. After hybridization (18 h), bead capture and washes, 15 cycles of PCR were performed with generic universal Illumina P7 and P5 primers. The final libraries were quantified by Qubit fluorometry (Thermo Fisher Scientific) and their size distribution analyzed by TapeStation electrophoresis (Agilent) prior to pooling and Illumina sequencing.

Pre-hybrid capture (HC) DNA libraries were sequenced on a HiSeqX, generating on average 10.9±2.8 million paired-end 151 bp reads. Pre-HC RNA libraries were sequenced on a NovaSeq, generating on average 95.8±35.5 million paired-end 151 bp reads. All post-HC libraries were run on an Illumina NovaSeq, generating on average 11.6±3.8 million paired-end 151 bp reads for post-HC DNA libraries, and 17.0±5.4 million paired-end 151 bp reads for post-HC RNA libraries, respectively. Sequences were uniformly downsampled to 6.67 million reads for DNA and 10 million reads for RNA for downstream analysis.

### Metagenomic Analysis of Human Stool Samples

For strain-level analysis of StrainGE, both pre- and post-HC sequence data were downsampled to 2 Gb (6,666,666 paired-end reads) per sample, in order to establish equivalent sequencing depths in the two sample types for comparisons. Duplicate reads were not de novo deduplicated because low PCR duplication was observed in the post-HC libraries for these libraries. Quality was assessed with FastQC and MultiQC. Downsampled data were then processed with Trim Galore and KneadData (huttenhower.sph.harvard.edu/kneaddata/) to remove low quality sequence, adapter content, and human contamination. StrainGST was used to determine strain identities, as well as their relative abundances and depth and breadth of coverage, using a database of 361 diverse Escherichia (including E. coli, Shigella, and other Escherichia species) RefSeq complete chromosomal references (i.e., excluding plasmid sequences) downloaded in June 2020.

Metagenomic assemblies were generated using the complete sequencing data, rather than downsampled data, in order to maximize assembly quality. Similar to the analysis of mock community data, digital normalization was performed with khmer, adapter trimming with Trim Galore, and assembly with MetaSPAdes. Assembly metrics were calculated as before, using GAEMR. Metrics reported in Table 3 were calculated based on only contigs assigned to E. coli using blastn. After assemblies were produced, a binning program, MetaBat2 (Kang, Dongwan D., Feng Li, Edward Kirton, Ashleigh Thomas, Rob Egan, Hong An, and Zhong Wang. 2019. “MetaBAT 2: An Adaptive Binning Algorithm for Robust and Efficient Genome Reconstruction from Metagenome Assemblies.” PeerJ 7 (July): e7359.), was used to produce metagenome-assembled genomes (MAGs). MAGs were analyzed with CheckM (Parks, Donovan H., Michael Imelfort, Connor T. Skennerton, Philip Hugenholtz, and Gene W. Tyson. 2015. “CheckM: Assessing the Quality of Microbial Genomes Recovered from Isolates, Single Cells, and Metagenomes.” Genome Research 25 (7): 1043-55.) to determine taxonomy and assembly completeness for MAGs that were classified as Enterobacteriaceae by CheckM (Table 4).

Taxonomic profiles for the metagenomes were calculated using Kraken2 (Wood, Derrick E., Jennifer Lu, and Ben Langmead. 2019. “Improved Metagenomic Analysis with Kraken 2.” Genome Biology 20 (1): 257.) and Bracken (Lu, Jennifer, Florian P. Breitwieser, Peter Thielen, and Steven L. Salzberg. 2017. “Bracken: Estimating Species Abundance in Metagenomics Data.” PeerJ Computer Science 3 (January): e104). E. coli and Shigella relative abundances were removed from the taxonomic table and relative abundances of the remaining taxa were renormalized to sum to 100%. Bray-Curtis Dissimilarity values were calculated with the R package vegan (cran.r-project.org/package=vegan). Principal Coordinate Analysis (PCoA) was performed with the R package ecodist (cran.r-project.org/package=ecodist).

### Metatranscriptomic Analysis of Human Stool Samples

In order to establish equivalent sequence depths for comparisons, both pre- and post-hybrid capture (HC) sequence data were downsampled to 3.5 Gb (10,000,000 paired-end reads) per sample. Quality was assessed with FastQC and MultiQC. Reads were processed with Trim Galore, SortMeRNA, and KneadData to remove adapter content, low quality sequence, human contamination, and ribosomal RNA content. Then, processed data were aligned with Bowtie2 (default parameters) to the reference of the strain(s) that was detected by StrainGST in the corresponding DNA sample. Properly paired reads with MQ≥5 were retained with samtools. Transcript abundance was calculated with htseq-count and then normalized for total sequencing depth per sample into Counts Per Million (CPM).

Transcripts were only considered in downstream analysis if they had expression levels of at least 10 CPM in either pre- or post-HC data. Transcripts that had 10 CPM or greater in pre-HC, but not post-HC, were considered drop-out transcripts. Transcripts that had 10 CPM or greater in post-HC, but not pre-HC, were considered novel transcripts.

### Statistical Analysis and Graphical Plotting

All statistical analysis and plotting was performed in R (Team, R. Core, and Others. 2012. “R: A Language and Environment for Statistical Computing.”) with the following libraries: ggplot2 (Wilkinson, Leland. 2011. “ggplot2: Elegant Graphics for Data Analysis by WICKHAM, H.” Biometrics. doi.org/10.1111/j.1541-0420.2011.01616.x), data.table (rdatatable.gitlab.io/data.table/), Rmisc (cran.r-project.org/package=Rmisc).

### Data Availability

Sequencing data for the pre-HC mock community was submitted to NCBI's Sequence Read Archive (SRA) under BioProject PRJNA685748 (BioSample SAMN17091845).

### Other Embodiments

From the foregoing description, it will be apparent that variations and modifications may be made to the invention described herein to adapt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1-109. (canceled)

110. A method of generating a set of probes, the method comprising:
(a) identifying a plurality of clusters of gene sequences derived from a set of genomes derived from organisms, and wherein the gene sequences within each cluster share at least about 25% nucleotide sequence identity;
(b) generating a set of probes, wherein each probe within the set comprises at least about 25 nucleotides and at least about 80% of said nucleotides are complementary to a target sequence present in the clusters of gene sequences, wherein the probes collectively target at least about 10% of all gene sequences in a gene cluster.

111. A method of characterizing a complex biological sample, the method comprising:
(a) contacting polynucleotides derived from the complex biological sample with the set of probes generated in claim 111, or a subset of such probes, under conditions that permit hybridization of the probes to the polynucleotides, thereby forming polynucleotide/probe complexes, wherein each probe is coupled to a binding member;
(b) contacting the polynucleotide/probe complexes with a capture molecule fixed to a solid support, wherein the capture molecule specifically binds the binding member of the probe, thereby enriching polynucleotides of the complex biological sample; and
(c) characterizing the enriched polynucleotides.

112. The method of claim 111, wherein the characterizing comprises sequencing, qPCR, fluorescent imaging, fluorescence activated cell sorting (FACS), genotyping array, or a NanoString assay.

113. A method for enrichment of polynucleotides derived from a complex biological sample, the method comprising:
(a) contacting polynucleotides derived from the complex biological sample with the set of probes of claim 110, or a subset thereof, under conditions that permit hybridization of the set of probes to the polynucleotides, wherein each probe is coupled to a binding member and/or to a solid support, thereby forming polynucleotide/probe complexes; wherein, if the probe is not coupled to a solid support, the method further comprises,
(b) contacting the polynucleotide/probe complex with a capture molecule fixed to a solid support, wherein the capture molecule specifically binds the binding member of the probe, thereby enriching the polynucleotides derived from the complex biological sample.

114. The method of claim 110, wherein the complex biological sample comprises polynucleotides derived from a host organism and an organism of interest, and wherein the enriched polynucleotides are derived from the organism of interest.

115. The method of claim 111, wherein the complex biological sample is an agricultural sample, biological sample, environmental sample, or food sample.

116. The method of claim 115, wherein the sample is collected from a surface of a medical device.

117. The method of claim 111, wherein the set of genomes comprises at least about 2, 5, 100, 250, or 500 genomes.

118. The method of claim 111, wherein the set of genomes are derived from a plurality of strains of a species.

119. The method of claim 111, wherein the organisms comprise a pathogen.

120. The method of claim 119, wherein the pathogen is selected from the group consisting of
Aerobacter, Aeromonas, Acinetobacter, Actinomyces israelii, Agrobacterium, Bacillus, Bacillus anthracis, Bacteroides, Bartonella, Bordetella, Bortella, Borrelia, Brucella, Burkholderia, Calymmatobacterium, Campylobacter, Citrobacter, Clostridium, Corynebacterium, Enterobacter, Enterococcus, Erysipelothrix rhusiopathiae, Escherichia, Faecalibacterium, Francisella, Fusobacterium micleatum, Gardnerella, Haemophilus, Hafnia, Helicobacter, Klebsiella, Legionella, Leptospira, Listeria, Morganella, Moraxella, Mycobacterium, Neisseria, Pasteurella, Proteus, Providencia, Pseudomonas, Rickettsia, Salmonella, Serratia, Shigella, Staphylococcus, Stentorophomonas, Streptococcus, Treponema, Xanthomonas, Vibrio, and Yersinia. spp.

121. The method of claim 111, wherein the organisms comprise a species belonging to the genus Akkermansia and/or Bifidobacterium.

122. The method of claim 120, wherein the organisms comprise a strain of E. coli selected from the group consisting of AIEC, DAEC, EAEC, EHEC, EIEC/Shigella, EPEC, ETEC, EXPEC, NMEC, SEPEC, ST131, and UPEC.

123. The method of claim 112, wherein the polynucleotides are enriched by a factor of at least about 2.

124. The method of claim 112, wherein the polynucleotide-probe complexes comprise a non-biased representation of sequence diversity in the complex biological sample.

125. The method of claim 111, wherein each probe shares at least about 90% nucleotide sequence identity across the length thereof with a gene sequence in at least one of the clusters.

126. The method of claim 112, wherein the subject has or has had an infection associated with the organisms.

127. The method of claim 126, wherein the infection is a chronic or recurring infection.

128. A method for generating a set of probes for use in pan-genomic or pan-transcriptomic sequencing of polynucleotides derived from organisms present in a microbiome, the method comprising:
(a) identifying a plurality of orthogroup clusters of gene sequences within a set of genomes derived from the organisms, wherein the organism are present in a microbiome;
(b) identifying a plurality of gene clusters within the orthogroup clusters, wherein the gene sequences within each of the gene clusters share at least about 80% nucleotide sequence identity; and
(c) generating a set of probes, wherein each probe within the set comprises from about 50 to about 85 nucleotides, wherein each probe is complementary to at least about 25 base pairs of a gene sequence present in the gene clusters identified in (b), wherein the probes collectively cover at least about 50% of all gene sequences in the gene clusters, and wherein the set excludes probes having 50 or more contiguous nucleotides that are identical to a set of reference sequences, and wherein coverage of each probe is determined using a cover extension of about 20 bp.

129. A polynucleotide array comprising the set of probes of claim 110.

130. A set of probes suitable for use in the method of claim 111, wherein the probes comprise a set of sequences each sharing at least 80% nucleotide sequence identity along a span of at least about 65 nucleotides to a sequence selected from SEQ ID NOs: 1 to 892415.

131. A set of enriched polynucleotide sequences obtained by the method of claim 111.

132. A panel of probes comprising probes generated according to the method of claim 110.

133. The panel of claim 132, wherein each probe comprises a unique molecular identifier, a bar code, a detectable moiety, and/or a binding member.
